(19) United States
(12) Patent Application Publication
(10) Pub. No.: US 2024/0316201 A1
(43) Pub. Date: Sep. 26, 2024

(54) ENGINEERED IMMUNE EFFECTOR CELLS EXPRESSING EXOGENOUSLY INTRODUCED CYTOKINES

(71) Applicant: NANJING LEGEND BIOTECH CO., LTD., Nanjing, Jiangsu (CN)

(72) Inventors: Xiaohu Fan, Edmonton (CA); Jie Mao, Nanjing (CN); Qiuchuan ZHUANG, Nanjing (CN); Changjiang Zhang, Nanjing (CN)

(21) Appl. No.: 18/276,703

(22) PCT Filed: Feb. 25, 2022
(86) PCT No.: PCT/CN2022/077964

§ 371 Date: Aug. 10, 2023

(30) Foreign Application Priority Data
Feb. 26, 2021 (WO) PCT/CN2021/078227

(51) Int. Cl. A61K 39/00; A61P 35/00; C07K 14/52; C07K 14/54; C07K 14/705; C07K 14/725; C07K 16/30; C12N 5/0783; C12N 15/86

(52) U.S. Cl. CPC A61K 39/464474; A61K 39/4611; A61K 39/4631; A61P 35/00; C07K 14/52; C07K 14/5434; C07K 14/7051; C07K 14/70517; C07K 14/70578; C07K 16/303; C12N 5/0636; C12N 15/86; C07K 2317/622; C07K 2319/03; C07K 2319/30; C12N 2740/15043

(57) ABSTRACT

An immune effector cell expressing an exogenously introduced p40 subunit of IL-12; an exogenously introduced ligand of CCR7 (such as CCL-19 and CCL-21); and a functional exogenous receptor (such as a chimeric antigen receptor) comprising an extracellular antigen binding domain, a transmembrane domain, and an intracellular signaling domain.

### 1. CROSS-REFERENCE TO RELATED APPLICATIONS

This application claims benefit of priority of International Patent Application No. PCT/CN2021/078227 filed on Feb. 26, 2021, the content of which is incorporated herein by reference in its entirety.

### 2. SEQUENCE LISTING

This application incorporates by reference a Sequence Listing submitted with this application as a text format, entitled “14651-039-228_SEQ_LISTING.txt,” created on Feb. 18, 2022 having a size of 145,395 bytes.

### 3. FIELD

The present disclosure relates to polypeptides comprising functional exogenous receptors (such as chimeric antigen receptors), engineered immune effector cells, and methods of use thereof. The present disclosure further relates to activation and expansion of cells for therapeutic uses, especially to chimeric antigen receptor-based T cell immunotherapies.

### 4. BACKGROUND

Adoptive transfer of engineered immune effector cells represents an emerging innovative therapeutic strategy. For instance, T cells engineered with chimeric antigen receptor (CAR) induce potent clinical response in patients with blood cancers, demonstrating promising superior prognosis comparing with conventional therapies. However, low in vivo survival efficiency, insufficient activation of endogenous immune cells, immunosuppressive micro-environment, T cell exhaustion, and difficulty infiltrating disease tissues are commonly cited reasons why CAR-T cells are challenging for certain diseases. Therefore, there is still a need in the art for improved constructs or engineered immune effector cells, e.g., CAR-T cells, for treating a disease or disorder.

### 5. SUMMARY

In one aspect, provided herein is an immune effector cell expressing an exogenously introduced p40 subunit of IL-12; an exogenously introduced ligand of CCR7 (e.g., CCL-19 and CCL-21); and a functional exogenous receptor comprising an extracellular antigen binding domain, a transmembrane domain, and an intracellular signaling domain.

In some embodiments, provided herein is an immune effector cell expressing an exogenously introduced p40 subunit of IL-12; an exogenously introduced CCL-19; and a functional exogenous receptor comprising an extracellular antigen binding domain, a transmembrane domain, and an intracellular signaling domain.

In some embodiments, provided herein is an immune effector cell expressing an exogenously introduced p40 subunit of IL-12; an exogenously introduced CCL-21; and a functional exogenous receptor comprising an extracellular antigen binding domain, a transmembrane domain, and an intracellular signaling domain.

In some embodiments, the p40 is a human p40 or a fragment or variant thereof. In some embodiments, the p40 comprises the amino acid sequence of SEQ ID NO: 5. In other embodiments, the p40 comprises an amino acid sequence having at least 75%, 80%, 90%, 91%, 92%, 93%, 94%, 95%, 96%, 97%, 98%, or 99% identity to the amino acid sequence of SEQ ID NO: 5. In some embodiments, the p40 polypeptide provided herein is secreted. In some embodiments, the p40 polypeptide provided herein is membrane bound (e.g., MB12).

In some embodiments, the CCL-19 is a human CCL-19 or a fragment or variant thereof. In some embodiments, the CCL-19 comprises the amino acid sequence of SEQ ID NO: 6. In other embodiments, the CCL-19 comprises an amino acid sequence having at least 75%, 80%, 90%, 91%, 92%, 93%, 94%, 95%, 96%, 97%, 98%, or 99% identity to the amino acid sequence of SEQ ID NO: 6.

In some embodiments, the CCL-21 is a human CCL-21 or a fragment or variant thereof. In some embodiments, the CCL-21 comprises the amino acid sequence of SEQ ID NO: 22. In other embodiments, the CCL-21 comprises an amino acid sequence having at least 75%, 80%, 90%, 91%, 92%, 93%, 94%, 95%, 96%, 97%, 98%, or 99% identity to the amino acid sequence of SEQ ID NO: 22.

In some embodiments, the functional exogenous receptor is a T cell receptor (TCR), a chimeric antigen receptor (CAR), a chimeric TCR (cTCR), or a T cell antigen coupler (TAC)-like chimeric receptor. In some embodiments, the functional exogenous receptor is a CAR.

In some embodiments, the transmembrane domain is derived from a molecule selected from the group consisting of CD8α, CD4, CD28, CD137, CD80, CD86, CD152 and PD1. In some embodiments, the transmembrane domain is from CD8α or CD28.

In some embodiments, the intracellular signaling domain comprises a primary intracellular signaling domain of an immune effector cell. In some embodiments, the primary intracellular signaling domain is from CD3ζ.

In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof. In some embodiments, the co-stimulatory signaling domain comprises a cytoplasmic domain of CD28 and/or a cytoplasmic domain of CD137.

In some embodiments, the functional exogenous receptor further comprises a hinge domain located between the C-terminus of the extracellular antigen binding domain and the N-terminus of the transmembrane domain. In some embodiments, the hinge domain is from CD8α.

In some embodiments, the functional exogenous receptor comprises a signal peptide located at the N-terminus of the polypeptide. In some embodiments, the signal peptide is from CD8α.

In some embodiments, the immune effector cell is a T cell.

In another aspect, provided herein is a polypeptide comprising an exogenously introduced p40 subunit of IL-12; an exogenously introduced ligand of CCR7 (such as CCL-19 and CCL-21); and a functional exogenous receptor comprising an extracellular antigen binding domain, a transmembrane domain, and an intracellular signaling domain.

In some embodiments, provided herein is a polypeptide comprising an exogenously introduced p40 subunit of IL-12; an exogenously introduced CCL-19; and a functional exogenous receptor comprising an extracellular antigen binding domain, a transmembrane domain, and an intracellular signaling domain.

In other embodiments, provided herein is a polypeptide comprising an exogenously introduced p40 subunit of IL-12; an exogenously introduced CCL-21; and a functional exogenous receptor comprising an extracellular antigen binding domain, a transmembrane domain, and an intracellular signaling domain.

In some embodiments, the p40 is a human p40 or a fragment or variant thereof. In some embodiments, the p40 comprises the amino acid sequence of SEQ ID NO: 5. In other embodiments, the p40 comprises an amino acid sequence having at least 75%, 80%, 90%, 91%, 92%, 93%, 94%, 95%, 96%, 97%, 98%, or 99% identity to the amino acid sequence of SEQ ID NO: 5. In some embodiments, the p40 polypeptide provided herein is secreted. In some embodiments, the p40 polypeptide provided herein is membrane bound (e.g., MB12).

In some embodiments, the CCL-19 is a human CCL-19 or a fragment or variant thereof. In some embodiments, the CCL-19 comprises the amino acid sequence of SEQ ID NO: 6. In other embodiments, the CCL-19 comprises an amino acid sequence having at least 75%, 80%, 90%, 91%, 92%, 93%, 94%, 95%, 96%, 97%, 98%, or 99% identity to the amino acid sequence of SEQ ID NO: 6.

In some embodiments, the CCL-21 is a human CCL-21 or a fragment or variant thereof. In some embodiments, the CCL-21 comprises the amino acid sequence of SEQ ID NO: 22. In other embodiments, the CCL-21 comprises an amino acid sequence having at least 75%, 80%, 90%, 91%, 92%, 93%, 94%, 95%, 96%, 97%, 98%, or 99% identity to the amino acid sequence of SEQ ID NO: 22.

In some embodiments, the functional exogenous receptor is a T cell receptor (TCR), a chimeric antigen receptor (CAR), a chimeric TCR (cTCR), or a T cell antigen coupler (TAC)-like chimeric receptor. In some embodiments, the functional exogenous receptor is a CAR.

In some embodiments, the transmembrane domain is derived from a molecule selected from the group consisting of CD8α, CD4, CD28, CD137, CD80, CD86, CD152 and PD1. In some embodiments, the transmembrane domain is from CD8α or CD28.

In some embodiments, the intracellular signaling domain comprises a primary intracellular signaling domain of an immune effector cell. In some embodiments, the primary intracellular signaling domain is from CD3ζ.

In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof. In some embodiments, the co-stimulatory signaling domain comprises a cytoplasmic domain of CD28 and/or a cytoplasmic domain of CD137.

In some embodiments, the functional exogenous receptor comprises a hinge domain located between the C-terminus of the extracellular antigen binding domain and the N-terminus of the transmembrane domain. In some embodiments, the hinge domain is from CD8α.

In some embodiments, the functional exogenous receptor further comprises a signal peptide located at the N-terminus of the polypeptide. In some embodiments, the signal peptide is from CD8α.

In some embodiments, the p40, the CCL-19, and the functional exogenous receptor are linked to each other via a peptide linker. In some embodiments, the p40, the CCL-21, and the functional exogenous receptor are linked to each other via a peptide linker. In some embodiments, the peptide linker is a 2A self-cleaving peptide optionally selected from a group consisting of F2A, E2A, P2A, T2A, and variants thereof. In some specific embodiments, the 2A self-cleaving peptide is a P2A fragment comprising the amino acid sequence of SEQ ID NO: 13. In some specific embodiments, the 2A self-cleaving peptide is a T2A fragment comprising the amino acid sequence of SEQ ID NO: 14.

In some embodiments, the p40 and the CCL-19 are present in a domain comprising the amino acid sequence of SEQ ID NO: 4. In other embodiments, the p40 and the CCL-19 are present in a domain comprising an amino acid sequence having at least 75%, 80%, 90%, 91%, 92%, 93%, 94%, 95%, 96%, 97%, 98%, or 99% identity to the amino acid sequence of SEQ ID NO: 4.

In some embodiments, the p40 and the CCL-21 are present in a domain comprising the amino acid sequence of SEQ ID NO: 20. In other embodiments, the p40 and the CCL-21 are present in a domain comprising an amino acid sequence having at least 75%, 80%, 90%, 91%, 92%, 93%, 94%, 95%, 96%, 97%, 98%, or 99% identity to the amino acid sequence of SEQ ID NO: 20.

In yet another aspect, provided herein is an isolated nucleic acid comprising a nucleic acid sequence encoding the polypeptide provided herein.

In yet another aspect, provided herein is an isolated nucleic acid comprising a first region encoding an exogenously introduced p40 subunit of IL-12; a second region encoding an exogenously introduced ligand of CCR7 (such as CCL-19 and CCL-21); and a third region encoding a functional exogenous receptor comprising an extracellular antigen binding domain, a transmembrane domain, and an intracellular signaling domain. In some embodiments, the ligand of CCR7 is CCL-19. In other embodiments, the ligand of CCR7 is CCL-21.

In yet another aspect, provided herein is a vector comprising the isolated nucleic acid provided herein.

In yet another aspect, provided herein is a method of making an immune effector cell comprising introducing into an immune cell (i) the nucleic acid or the vector provided herein; or (ii) a composition comprising two or more nucleic acids each encoding one or two of p40 subunit of IL-12, CCL-19; and a functional exogenous receptor, or a composition comprising two or more nucleic acids each encoding one or two of p40 subunit of IL-12, CCL-21; and a functional exogenous receptor.

In another aspect, provided herein is an immune effector cell produced according the method provided herein.

In yet another aspect, provided herein is a pharmaceutical composition, comprising the immune effector cell, the polypeptide, the nucleic acid, or the vector provided herein, and a pharmaceutically acceptable carrier.

In yet another aspect, provided herein is a method of treating a disease or disorder in a subject, comprising administering to the subject an effective amount of the pharmaceutical composition provided herein.

### 6. BRIEF DESCRIPTION OF THE FIGURES

FIG. 1 shows the structures of H93 CAR, H93M CAR, and H93P CAR. SP refers to the signal peptide. TM refers to the transmembrane domain. TAA Binder refers to antigen binding domain or the binder of a tumor-associated antigen (i.e. anti-GPC3 scFv).

FIG. 2 shows the positive rates of H93 CAR-T cells and H93M CAR-T cells expressing humanized anti-GPC3 scFv CAR. UnT refers to T cells un-transduced with CAR.

FIGS. 3A-3B show the production of IL-23 (FIG. 3A) and CCL-19 (FIG. 3B) proteins of CAR-T cells cultured in normal condition (RPMI-1640+300 IU/mL IL-2).

FIGS. 4A-4B show the expansion of CAR-T cells in the re-challenge assay. Cells were stimulated overnight with PLC/PRF/5 cells at a 1:1 E/T ratio every 2 days. The expansion fold (FIG. 4A) and viability (FIG. 4B) of T cells were recorded at the end of each round.

FIGS. 5A-5C show in vitro cytotoxicity (FIG. 5A), TNF-α release level (FIG. 5B) and IFN-γ release level (FIG. 5C) of CAR-T cells against GPC3 positive cell line with 1:1 FT ratio after being treated with different stimulation rounds in the re-challenge assay.

FIG. 6 shows the positive ratio of CAR-T cells after being treated with different stimulation rounds in the re-challenge assay.

FIGS. 7A-7D show the expression of PD-1 (FIG. 7A and FIG. 7B) and LAG3 (FIG. 7C and FIG. 7D) in CAR-T cells 24 hours after each stimulation round in the re-challenge assay.

FIGS. 8A-8B show cell migration assay of CAR-T cells performed by 96-well transwell chambers. Different CAR-T cells were co-cultured with PLC/PRF/5 cells at 1:1 E/F ratio for 30 hours and the cell culture supernatant was collected and added to lower chamber. The T cell number of lower chamber migrating from the upper chamber after 4, 6, 8 hours were quantified (FIG. 8A), as well as the concentration of CCL-19 in co-culture supernatant (FIG. 8B).

FIGS. 9A-9D show the anti-tumor effects of CAR-T cells in NCG mouse xenograft model. NCG mice were inoculated subcutaneously with Huh7 cells, and treated with CAR-T cells at 0.2 M dosage and 0.6 M dosage (i.v.) (n=4 mice per group). The volume of tumors (FIG. 9A and FIG. 9B), CAR copy number (FIG. 9C and FIG. 9D) in genomic DNA of NCG mice peripheral blood were assessed.

FIGS. 10A-10B show the weight change of mice treated with CAR-T cells at 0.2 M (FIG. 10A) and 0.6 M (FIG. 10B) dosages (n=4 mice per group) in NCG mouse xenograft model.

FIGS. 11A-11D show the level of IL-23 protein (FIG. 11A and FIG. 11B) and IFN-γ protein (FIG. 11C and FIG. 11D) in NCG mice peripheral blood after treated with 0.2 M or 0.6 M dosage CAR-T cells (n=3 mice per group).

FIG. 12 shows the infiltration of T cells in tumor tissues of mice treated with 0.2 M UnT, H93 CAR-T cells or H93M CAR-T cells at the endpoint of the animal experiment (n=3) in NCG mouse xenograft model. Tumor tissues were fixed with formalin, embedded in paraffin and used for immunohistochemistry (IHC) to detect the infiltration of T cells which were stained by 3,3′-Diaminobenzidine (DAB). The images were obtained by 3D HISTECH (DRNJIER). The right column shows the enlarged area of the black rectangle.

FIG. 13 shows the recruitment of mouse macrophage and mouse dendritic cell in tumor tissues after treated with 0.2 M UnT, H93 CAR-T cells or H93M CAR-T cells in NCG mouse xenograft model. At the endpoint of the animal experiment, tumor tissues were fixed with formalin, embedded in paraffin and used for immunohistochemistry (IHC). Macrophage with CD68 (left column) and dendritic cell with CD11c (right column) were stained by 3,3′-Diaminobenzidine (DAB). The images were obtained by 3D HISTECH (DRNJIER).

FIG. 14 shows the positive rates of H93 CAR-T cells, H93M CAR-T cells and H93P CAR-T cells expressing humanized anti-GPC3 scFv CAR.

FIGS. 15A-15B show the results of a re-challenge assay. The expansion fold CAR-T cells were recorded at the end of each round (FIG. 15A). FIG. 15B shows in vitro cytotoxicity of CAR-T cells against GPC3 positive cell line at total T cells:target cells ratio of 1:2 after treated with different stimulation rounds in re-challenge assay.

FIG. 16 shows cell migration assay of CAR-T cells performed by 96-well transwell chambers. Different CAR-T cells were co-cultured with PLC/PRF/5 cells at 1:1 E/T ratio for 30 hours and the cell culture supernatant were collected and added to lower chamber. The T cell number of lower chamber migrating from the upper chamber after 2, 4 and 6 hours were quantified.

FIGS. 17A-17B show the anti-tumor effects of CAR-T cells in NCG mouse xenograft model. NCG mice were inoculated subcutaneously with GPC3+ Hep3B cells, and infused with CAR-T cells at two doses of 0.3 M CAR+ T cells/mouse and 0.8 M CAR+ T cells/mouse (i.v.) (n=4 mice per group).

FIGS. 18A-18C show the anti-tumor effects of CAR-T cells in NCG mouse xenograft model. NCG mice were inoculated subcutaneously with Hep3B cells, and treated with H93 CAR-T cells, H93M CAR-T cells, H93IL12p40 CAR-T cells or H93CCL19 CAR-T cells at 0.3 M dosage (i.v.) (n=4 mice per group). The volume of tumors (FIG. 18A), the rates of CD3 positive cells (FIG. 18B) in NCG mice peripheral blood and bodyweight (FIG. 18C) were assessed.

FIGS. 19A-19D show the anti-tumor effects of CAR-T cells in C57BL/6 mouse xenograft model. C57BL/6 mice were inoculated subcutaneously with LL/2-hGPC3 cells, and treated with musH93 CAR-T cells, musH93M CAR-T cells, musH93IL 12p40 CAR-T cells, and musH93CCL19 CAR-T cells at 10M, 6M, 6M and 6M dosages, respectively (i.v.) (n=5 mice per group). The volume of tumors (FIG. 19A), the ratio of CAR⁺CD3⁺ (FIG. 19B) in C57BL/6 mice peripheral blood and bodyweight (FIG. 19C) were assessed, and the percent survival (%) was calculated (FIG. 19D).

FIGS. 20A-20B show the production of IL-23 (FIG. 20A) and CCL-19 (FIG. 20B) proteins of 261 CAR-T cells and 261M CAR-T cells after co-cultured with CLDN18.2+ NUGC4 cells overnight.

FIGS. 21A-21C show the results of re-challenge assay of 261 CAR-T cells and 261M CAR-T cells. The comparison of expansion fold of CAR-T cells in traditional condition (10%1640+300 IU IL-2) (FIG. 21A) and in re-challenged assay (FIG. 21B) indicated that 261M CAR-T cells after multiple challenged by NUGC4 cells showed antigen dependent expansion. FIG. 21C shows cell viability of CAR-T cells after treated with multiple stimulation rounds in re-challenge assay.

FIGS. 22A-22D show the anti-tumor effects of 261 CAR-T cells and 261M CAR-T cells in NCG mouse xenograft model. NCG mice were inoculated subcutaneously with NUGC4 cells, and treated with CAR-T cells at 0.5 M dosage (i.v.) (n=4 mice per group). The volume of tumors (FIGS. 22A and B), the ratio of CD3 positive cells (FIG. 22C) in NCG mice peripheral blood and bodyweight (FIG. 22D) were assessed.

FIGS. 23A-23B show the anti-tumor effects of mus261M CAR-T cells and mus261-719 CAR-T cells in C57BL/6 mouse xenograft model. C57BL/6 mice were inoculated subcutaneously with LL/2-hCLDN18.2 cells, and treated with mouse CAR-T cells at 10M dosage (i.v.) (n=4 mice per group). The volume of tumors (FIG. 23A) and bodyweight (FIG. 23B) were assessed.

FIGS. 24A-24B show the CAR positive rates (FIG. 24A) and IL 12p40 positive rates (FIG. 24B) of H93 CAR-T cells, H93M CAR-T cells and H93M-MB12 CAR-T cells.

FIGS. 25A-25B show the production of IL 12p40 (FIG. 25A), IL-23 (FIG. 25B), CCL-19 (FIG. 25C), IL-12 (FIG. 25D) proteins of H93 CAR-T cells, H93M CAR-T cells and H93M-MB12 CAR-T cells after stimulation with anti-CD3/CD28 beads for overnight.

FIGS. 26A-26B show cell migration assay of H93 CAR-T cells, H93M CAR-T cells and H93M-MB12 CAR-T cells performed by 96-well transwell chambers. Different CAR-T cells were co-cultured with PLC/PRF/5 cells at 1:1 E/F ratio for 24 hours and the cell culture supernatant was collected and added to lower chamber. The T cell number of lower chamber migrating from the upper chamber after 2, 4, 6 hours were quantified (FIG. 26A), as well as the concentration of CCL-19 in co-culture supernatant (FIG. 26B).

FIGS. 27A-27C show the results of a re-challenge assay of H93 CAR-T cells, H93M CAR-T cells and H93M-MB12 CAR-T cells. FIG. 27A and FIG. 27B show the expansion fold of CAR-T cells in traditional condition (10%1640+300 IU IL-2) and in re-challenged assay. FIG. 27C shows CAR positive change of CAR-T cells after treated with multiple stimulation rounds in re-challenge assay.

FIGS. 28A-28B show the secretion of cytokines in vitro, TNF-α, release level (FIG. 28A) and IFN-γ release level (FIG. 28B) of H93 CAR-T cells, H93M CAR-T cells and H93M-MB12 CAR-T cells after being treated with different stimulation rounds in the re-challenge assay.

### 7. DETAILED DESCRIPTION

The present disclosure is based, in part, on the surprising finding of improved functions and properties of engineered immune cells expressing one or more functional exogenous receptor(s) (such as a CAR) and exogenously introduced p40 and CCR7 ligand (such as CCL-19 and CCL-21).

### 7.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, single domain antibodies (e.g., VHH) and fragments thereof (e.g., domain antibodies). An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Single domain antibody” or “sdAb” as used herein refers to a single monomeric variable antibody domain and which is capable of antigen binding. Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; VHHs derived from such other species are within the scope of the disclosure. In some embodiments, the single domain antibody (e.g., VHH) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a chimeric antigen receptor).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (koff) to association rate (kon) of a binding molecule (e.g., an antibody) to a monovalent antigen (koff/kon) is the dissociation constant KD, which is inversely related to affinity. The lower the KD value, the higher the affinity of the antibody. The value of KD varies for different complexes of antibody and antigen and depends on both kon and koff. The dissociation constant KD for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by FACS analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (KD) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise a single domain antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boerner et al., J. Immunol. 147(1):86-95 (1991); and van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Brüggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid isomerization or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for p and a isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L 1, L2 or L3) within the non-framework region of the antibody VL β-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27(1):55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-70 (2001). Correspondence between the numbering system, including, for example, the Kabat numbering and the IMGT unique numbering system, is well known to one skilled in the art (see. e.g., Kabat, supra; Chothia and Lesk, supra; Martin, supra; Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26--H32 . . . 34 | H30--H35B | H27--H38 |
| | (Kabat | | | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 or | H93--H101 | H105-H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR as set forth in a specific VH or VHH” includes any CDR1 as defined by the exemplary CDR numbering systems described above, but is not limited thereby. Once a variable region (e.g., a VHH, VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies (e.g., single domain antibodies), diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody comprising a single chain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “functional exogenous receptor” as used herein, refers to an exogenous receptor (e.g., TCR such as a recombinant or engineered TCR, cTCR, TAC-like chimeric receptor, or CAR) that retains its biological activity after being introduced into an immune effector cell such as a T cell. The biological activity includes but are not limited to the ability of the exogenous receptor in specifically binding to a molecule, properly transducing downstream signals, such as inducing cellular proliferation, cytokine production and/or performance of regulatory or cytolytic effector functions.

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” In some embodiments, the CAR comprises an extracellular antigen binding domain specific for one or more antigens (such as tumor antigens), a transmembrane domain, and an intracellular signaling domain of a T cell and/or other receptors. “CAR-T cell” or “CAR-T” refers to a T cell that expresses a CAR.

The term “recombinant or engineered TCR” as used herein is included as a kind of functional exogenous receptor provided herein, and refers to peptide expressed into an immune cell. The functions of recombinant or engineered TCR may include for example redirecting immune activity of the immune cell against a desired type of cells, such as cancer and infected cells having specific markers at their surface. It can replace or be-co-expressed with the endogenous TCR. In some embodiments, such recombinant TCR are single-chain TCRs comprising an open reading frame where the variable Vα and Vβ domains are paired with a protein linker. This involves the molecular cloning of the TCR genes known to be specific for an antigen of choice. These chains are then introduced into T cells usually by means of a retroviral vector. Consequently, expression of the cloned TCRα and TCRβ genes endows the transduced T cell with a functional specificity determined by the pairing of these new genes. A component of a recombinant or engineered TCR is any functional subunit of a TCR, such as a recombined TCRα and TCRβ, which is encoded by an exogenous polynucleotide sequence introduced into the cell.

In some embodiments, the functional exogenous receptor provided herein is a chimeric TCR (cTCR), which has both antigen-binding and T-cell activating functions. For example, a cTCR can comprise: (a) an extracellular ligand binding domain comprising an antigen-binding fragment (e.g., sdAb, scFv) that specifically recognizes one or more epitopes of a tumor antigen (e.g., GPC3, Claudin18.2); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., all CD3ε). In some embodiments, the first, second, and third TCR subunits are different. In some embodiments, the cTCR further comprises a hinge domain located between the C-terminus of the extracellular ligand binding domain and the N-terminus of the transmembrane domain. In some embodiments, the hinge domain is derived from CD8α. In some embodiments, the cTCR further comprises a signal peptide located at the N-terminus of the cTCR, such as a signal peptide derived from CD8α.

In some embodiments, the functional exogenous receptor is a T cell antigen coupler (TAC), e.g., comprising: (a) an extracellular ligand binding domain comprising an antigen-binding fragment (e.g., sdAb, scFv) that specifically recognizes one or more epitopes of a tumor antigen (e.g., GPC3, Claudin18.2); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28. In some embodiments, the first, second, and third TCR co-receptors are the same. In some embodiments, the first, second, and third TCR co-receptors are different. In some embodiments, the TAC further comprises a hinge domain located between the C-terminus of the extracellular ligand binding domain and the N-terminus of the transmembrane domain. In some embodiments, the hinge domain is derived from CD8α. In some embodiments, the TAC further comprises a signal peptide located at the N-terminus of the TAC, such as a signal peptide derived from CD8α. In some embodiments, the extracellular ligand binding domain is at N-terminal of the extracellular TCR binding domain. In some embodiments, the extracellular ligand binding domain is at C-terminal of the extracellular TCR binding domain.

In some embodiments, the functional exogenous receptor is a TAC-like chimeric receptor, e.g., comprising: (a) an extracellular ligand binding domain comprising an antigen-binding fragment (e.g., sdAb, scFv) that specifically recognizes one or more epitopes of a tumor antigen (e.g., GPC3, Claudin18.2); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRS, CD3ε, CD3γ, and CD3δ. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, the extracellular ligand binding domain is at N-terminal of the extracellular TCR binding domain. In some embodiments, the extracellular ligand binding domain is at C-terminal of the extracellular TCR binding domain. In some embodiments, the TAC-like chimeric receptor further comprises a hinge domain located between the C-terminus of the extracellular ligand binding domain and the N-terminus of the transmembrane domain. In some embodiments, the hinge domain is derived from CD8α. In some embodiments, the TAC-like chimeric receptor further comprises a signal peptide located at the N-terminus of the TAC-like chimeric receptor, such as a signal peptide derived from CD8α.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a single chain antibody or an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a CAR described herein is a nucleic acid molecule that is identified and separated from at least one contaminant nucleic acid molecule with which it is ordinarily associated in the environment in which it was produced.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

“Allogeneic” refers to a graft derived from a different individual of the same species.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of engineered immune effector cells or a therapeutic molecule comprising an agent and the engineered immune effector cells or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., a cancer).

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 7.2. Engineered Immune Effector Cells

As shown in Section 8 below, introducing a specific combination of p40 subunit of IL-12 and a ligand of CCR7 (e.g., CCL-19 and CCL-21) into immune effector cells expressing one or more functional exogenous receptor(s) can significantly improves properties/functions of the immune effector cells. Thus, in one aspect, provided herein are host cells (such as immune effector cells) comprising exogenously introduced p40 subunit of IL-12 and a ligand of CCR7 (e.g., CCL-19 and CCL-21), wherein the host cells express one or more functional exogenous receptor(s).

CCR7 (C-C chemokine receptor type 7) is a protein that in humans is encoded by the CCR7gene. CCR7 is also known as BLR2, CC-CKR-7, CCR-7, CD197, CDw197, CMKBR7, EBI1, and C-C motif chemokine receptor 7. In some embodiments, CCR7 provided herein is identified as OMIM: 600242; MGI: 103011; HomoloGene: 1387; and/or GeneCards: CCR7. Two natural ligands have been identified for this receptor: CCL-19 and CCL-21. The ligand of CCR7 provided herein can be a natural ligand of CCR7 of any species. In other embodiments, the ligand of CCR7 provided herein can be an artificial or synthetic ligand of CCR7 of any species.

The functional exogenous receptors can be, for example, chimeric antigen receptor (CAR), engineered T cell receptor (TCR), chimeric TCR (cTCR), and T cell antigen coupler (TAC)-like chimeric receptor. In some embodiments, the functional exogenous receptor is a CAR. In some embodiments, the functional exogenous receptor is a TCR. In some embodiments, the functional exogenous receptor is cTCR. In yet other embodiments, the functional exogenous receptor is a TAC. Any immune effector cell that can perform immune effector functions may be used in the present invention, including but not limited to, peripheral blood mononuclear cells (PBMC), natural killer (NK) cells, monocytes, cytotoxic T cells, neutrophils, and eosinophils.

In some embodiments, provided herein are host cells, such as immune effector cells, comprising any one of the polypeptides, polynucleotides, or vectors described herein, for example, as provided in the following sections.

In some specific embodiments, provided herein is a CAR-T cell expressing exogenously introduced p40 and CCL-19. In some specific embodiments, provided herein is a TCR-T cell expressing exogenously introduced p40 and CCL-19. In some specific embodiments, provided herein is a TAC-T cell expressing exogenously introduced p40 and CCL-19.

In some specific embodiments, provided herein is a CAR-T cell expressing exogenously introduced p40 and CCL-21. In some specific embodiments, provided herein is a TCR-T cell expressing exogenously introduced p40 and CCL-21. In some specific embodiments, provided herein is a TAC-T cell expressing exogenously introduced p40 and CCL-21.

### 7.2.1. P40, CCL-19, and CCL-21

In some embodiments, the present engineered immune effector cells comprise a specific combination of exogenously introduced p40 subunit of IL-12 or a variant thereof and CCL-19 or a variant thereof. In other embodiments, the present engineered immune effector cells comprise a specific combination of exogenously introduced p40 subunit of IL-12 or a variant thereof and CCL-21 or a variant thereof.

As used herein, p40 is subunit beta of interleukin 12, also known as IL-12B, natural killer cell stimulatory factor 2, cytotoxic lymphocyte maturation factor p40, or interleukin-12 subunit p40, and is a protein that in humans is encoded by the IL 12B gene. P40 is a common subunit of interleukin 12 and interleukin 23. In some embodiments, p40 is identified as OMIM: 161561, MGI: 96540, HomoloGene: 1648, and/or GeneCards: IL12B. The p40 as used herein includes p40 from any species. The p40 as used herein also includes any p40 variants that essentially retain at least one of its biological activities (e.g., retains at least 80% of one of its biological activities). In some embodiments, p40 provided herein is a human p40 or a fragment or a variant thereof. In a specific embodiment, the p40 provided herein comprises the amino acid sequence of SEQ ID NO: 5. In some embodiments, the p40 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 5. For example, in some embodiments, p40 provided herein comprises an amino acid sequence having about 80% sequence identity to SEQ ID NO: 5, and retains at least 80% of a biological activity of a p40 comprising the amino acid sequence of SEQ ID NO: 5. In some embodiments, the p40 polypeptide provided herein is secreted. In some embodiments, the p40 polypeptide provided herein is membrane bound (e.g., MB12).

The polypeptide of p40 provided herein is membrane-bound formed by any technique known in the art. In some embodiments, the p40 polypeptide is bound to the membrane via a membrane anchoring domain. In some embodiments, the p40 polypeptide is bound to the membrane via a transmembrane domain. Exemplary transmembrane domains include, but not limited to, the transmembrane domain of an alpha, beta or zeta chain of a T-cell receptor, CD28, CD3 epsilon, CD45, CD4, CD5, CD8, B7, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, CD8α, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the p40 polypeptide is membrane bound further comprising a second polypeptide fragment. In some embodiments, the second polypeptide fragment is selected from the group consisting of CD28, CD8α, OX40 and B7. In some embodiments, the p40 fragment and the second polypeptide fragment are linked to each other with a peptide linker. In some embodiments, the linker peptide is a flexible linker. Exemplary flexible linkers include but not limited to glycine polymers (G)n, glycine-serine polymers (including, for example, (GSGGS)n (SEQ ID NO: 46), (GGGS)n (SEQ ID NO: 47), and (GGGGS)n (SEQ ID NO: 48), where n is an integer of at least one), glycine-alanine polymers, alanine-serine polymers, and other flexible linkers known in the art. In some embodiments, the membrane bound p40 polypeptide provided herein comprises from N terminus to C terminus: a signal peptide, p40, a hinge and transmembrane domain derived from CD8α, and optionally an intracellular signaling domain from CD8α. In a specific embodiment, the membrane bound p40 polypeptide provided herein comprises the amino acid sequence of SEQ ID NO: 37. In some embodiments, the membrane bound p40 polypeptide further comprising a third polypeptide fragment. In some embodiments, the third polypeptide fragment is a dominant negative receptor (DNR). In some embodiments, the third polypeptide fragment is a TGFβRII DNR. In some embodiments, the membrane bound p40 polypeptide provided herein comprises from N terminus to C terminus: a signal peptide, p40, TGFβRII DNR, a hinge and transmembrane domain derived from CD8α, and optionally an intracellular signaling domain from CD8α.

Chemokine (C-C motif) ligand 19 (CCL-19) is a protein that in humans is encoded by the CCL19 gene, which also is known as CKb11, ELC, MIP-3b, MIP3B, SCYA19, and C-C motif chemokine ligand 19. In some embodiments, CCL-19 is identified as OMIM: 602227; MGI: 5434459; HomoloGene: 4569; and/or GeneCards: CCL19. The CCL-19 as used herein includes CCL-19 from any species. The CCL-19 as used herein also includes any CCL-19 variants that essentially retain at least one of its biological activities (e.g., retains at least 80% of one of its biological activities). In some embodiments, CCL-19 provided herein is a human CCL-19 or a fragment or a variant thereof. In a specific embodiment, the CCL-19 provided herein comprises the amino acid sequence of SEQ ID NO: 6. In some embodiments, the CCL-19 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 6. For example, in some embodiments, CCL-19 provided herein comprises an amino acid sequence having about 80% sequence identity to SEQ ID NO: 6, and retains at least 80% of a biological activity of a CCL-19 comprising the amino acid sequence of SEQ ID NO: 6.

Chemokine (C-C motif) ligand 21 (CCL-21) is a cytokine belonging to the CC chemokine family. CCL-21 is also known as 6Ckine, CKb9, ECL, SCYA21, SLC, TCA4, and C-C motif chemokine ligand 21. In some embodiments, CCL-21 is identified as OMIM: 602737; MGI: 1349183; HomoloGene: 2247; and/or GeneCards: CCL21. The CCL-21 as used herein includes CCL-21 from any species. The CCL-21 as used herein also includes any CCL-21 variants that essentially retain at least one of its biological activities (e.g., retains at least 80% of one of its biological activities). In some embodiments, CCL-21 provided herein is a human CCL-21 or a fragment or a variant thereof. In a specific embodiment, the CCL-21 provided herein comprises the amino acid sequence of SEQ ID NO: 22. In some embodiments, the CCL-21 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 22. For example, in some embodiments, CCL-21 provided herein comprises an amino acid sequence having about 80% sequence identity to SEQ ID NO: 22, and retains at least 80% of a biological activity of a CCL-21 comprising the amino acid sequence of SEQ ID NO: 22.

In some embodiments, the p40 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 5; and the CCL-19 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 6. In a specific embodiment, the p40 provided herein comprises the amino acid sequence of SEQ ID NO: 5; and the CCL-19 provided herein comprises the amino acid sequence of SEQ ID NO: 6.

In some embodiments, the p40 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 5; and the CCL-21 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 22. In a specific embodiment, the p40 provided herein comprises the amino acid sequence of SEQ ID NO: 5; and the CCL-21 provided herein comprises the amino acid sequence of SEQ ID NO: 22.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In some embodiments, amino acid sequence modification(s) or variation(s) of the p40, CCL-19 and CCL-21 described herein are contemplated. Variations may be a substitution, deletion, or insertion of one or more codons encoding the polypeptide that results in a change in the amino acid sequence as compared with the original polypeptide.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental polypeptides.

The polypeptides generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties.

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. For example, any cysteine residue not involved in maintaining the proper conformation of the peptide also may be substituted, for example, with another amino acid, such as alanine or serine, to improve the oxidative stability of the molecule and to prevent aberrant crosslinking. Non-conservative substitutions will entail exchanging a member of one of these classes for another class.

Amino acid sequence insertions include amino- and/or carboxyl-terminal fusions ranging in length from one residue to polypeptides containing a hundred or more residues, as well as intrasequence insertions of single or multiple amino acid residues. The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the polypeptide variant DNA.

### 7.2.2. Chimeric Antigen Receptors

The present engineered immune effector cells express one or more functional exogenous receptors. Any functional exogenous receptors are included in the present disclosure. Chimeric antigen receptor (CAR) is described in more detail below only as an exemplary functional exogenous receptor provided herein, but does not limit the scope of the present disclosure.

In some embodiments, the CAR in the present immune effector cells comprises a polypeptide comprising: (a) an extracellular antigen binding domain; (b) a transmembrane domain; and (c) an intracellular signaling domain, each of which and additional regions are described in more detail below.

### Extracellular Antigen Binding Domain

The extracellular antigen binding domain of the CARs described herein comprises one or more antigen binding domains. In some embodiments, the extracellular antigen binding domain of the CAR provided herein is monospecific. In other embodiments, the extracellular antigen binding domain of the CAR provided herein is multispecific. In other embodiments, the extracellular antigen binding domain of the CAR provided herein is monovalent. In other embodiments, the extracellular antigen binding domain of the CAR provided herein is multivalent. In some embodiments, the extracellular antigen binding domain comprises two or more antigen binding domains which are fused to each other directly via peptide bonds, or via peptide linkers.

In some embodiments, the extracellular antigen binding domain comprises an antibody or a fragment thereof. For example, the binding domain may be derived from monoclonal antibodies (including agonist, antagonist, neutralizing antibodies, full length or intact monoclonal antibodies), antibody with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, single domain antibodies and fragments thereof (e.g., domain antibodies). An antibody can be human, humanized, chimeric and/or affinity matured, as well as an antibody from other species, for example, mouse, rabbit, llama, etc. In some embodiments, the antibody include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)2 fragments, F(ab′)2 fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

In a specific embodiment, the extracellular antigen binding domain of the present CARs comprise a single-chain Fv (sFv or scFv). ScFvs are antibody fragments that comprise the VH and VL antibody domains connected into a single polypeptide chain. Preferably, the scFv polypeptide further comprises a polypeptide linker between the VH and VL domains which enables the sFv to form the desired structure for antigen binding. See Pluckthun in The Pharmacology of Monoclonal Antibodies, vol. 113, Rosenburg and Moore eds., Springer-Verlag, New York, pp. 269-315 (1994).

In another specific embodiment, the extracellular antigen binding domain of the present CARs comprises one or more single domain antibodies (sdAbs). The sdAbs may be of the same or different origins, and of the same or different sizes. Exemplary sdAbs include, but are not limited to, heavy chain variable domains from heavy-chain only antibodies (e.g., VHH or VNAR), binding molecules naturally devoid of light chains, single domains (such as VH or VL) derived from conventional 4-chain antibodies, humanized heavy-chain only antibodies, human single domain antibodies produced by transgenic mice or rats expressing human heavy chain segments, and engineered domains and single domain scaffolds other than those derived from antibodies. Any sdAbs known in the art or developed by the present disclosure, including the single domain antibodies described above in the present disclosure, may be used to construct the CARs described herein. The sdAbs may be derived from any species including, but not limited to mouse, rat, human, camel, llama, lamprey, fish, shark, goat, rabbit, and bovine. Single domain antibodies contemplated herein also include naturally occurring single domain antibody molecules from species other than Camelidae and sharks.

In some embodiments, the sdAb is derived from a naturally occurring single domain antigen binding molecule known as heavy chain antibody devoid of light chains (also referred herein as “heavy chain only antibodies”). Such single domain molecules are disclosed in WO 94/04678 and Hamers-Casterman, C. et al., Nature 363:446-448 (1993), for example. For clarity reasons, the variable domain derived from a heavy chain molecule naturally devoid of light chain is known herein as a VHH to distinguish it from the conventional VH of four chain immunoglobulins. Such a VHH molecule can be derived from antibodies raised in Camelidae species, for example, camel, llama, vicuna, dromedary, alpaca and guanaco. Other species besides Camelidae may produce heavy chain molecules naturally devoid of light chain, and such VHHs are within the scope of the present disclosure. In addition, humanized versions of VHHs as well as other modifications and variants are also contemplated and within the scope of the present disclosure. In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov, Protein Sci. 14:2901-2909 (2005).

In some embodiments, naturally occurring VHH domains against a particular antigen or target, can be obtained from (naïve or immune) libraries of Camelid VHH sequences. Such methods may or may not involve screening such a library using said antigen or target, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from (naïve or immune) VHH libraries may be used, such as VHH libraries obtained from (naïve or immune) VHH libraries by techniques such as random mutagenesis and/or CDR shuffling, as for example described in WO 00/43507.

In some embodiments, the sdAb is recombinant, CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacing or substitution of one or more amino acid residues in the amino acid sequence of a (naturally occurring) VH domain from a conventional 4-chain antibody by one or more of the amino acid residues that occur at the corresponding position(s) in a VHH domain of a heavy chain antibody. This can be performed in a manner known in the field, which will be clear to the skilled person. Such “camelizing” substitutions are preferably inserted at amino acid positions that form and/or are present at the VH-VL interface, and/or at the so-called Camelidae hallmark residues, as defined herein (see for example WO 94/04678, Davies and Riechmann FEBS Letters 339: 285-290 (1994); Davies and Riechmann, Protein Engineering 9 (6): 531-537 (1996); Riechmann, J. Mol. Biol. 259: 957-969 (1996); and Riechmann and Muyldermans, J. Immunol. Meth. 231: 25-38 (1999)).

In some embodiments, the sdAb is a human single domain antibody produced by transgenic mice or rats expressing human heavy chain segments. See, e.g., US20090307787, U.S. Pat. No. 8,754,287, US20150289489, US20100122358, and WO2004049794.

In some embodiments, the single domain antibodies are generated from conventional four-chain antibodies. See, for example, EP 0 368 684; Ward et al., Nature, 341 (6242): 544-6 (1989); Holt et al., Trends Biotechnol., 21(11):484-490 (2003); WO 06/030220; and WO 06/003388.

In some embodiments, the extracellular antigen binding domain comprises humanized antibodies or fragment thereof. A humanized antibody can comprise human framework region and human constant region sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 93/17105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

A deimmunized antibody is an antibody in which T-cell epitopes have been removed. Methods for making deimmunized antibodies have been described. See, e.g., Jones et al., Methods Mol Biol. 2009; 525:405-23, xiv, and De Groot et al., Cell. Immunol. 244:148-153(2006)). Deimmunized antibodies comprise T-cell epitope-depleted variable regions and human constant regions. Briefly, VH and VL of an antibody are cloned and T-cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

In certain embodiments, the extracellular antigen binding domain comprises multiple binding domains. In some embodiments, the extracellular antigen binding domain comprises multispecific antibodies or fragments thereof, e.g., an extracellular antigen binding domain comprising multiple binding domains (e.g., multiple scFvs) in tandem. In other embodiments, the extracellular antigen binding domain comprises multivalent antibodies or fragments thereof. The term “specificity” refers to selective recognition of an antigen binding protein for a particular epitope of an antigen. The term “multispecific” as used herein denotes that an antigen binding protein has two or more antigen-binding sites of which at least two bind different antigens. The term “valent” as used herein denotes the presence of a specified number of binding sites in an antigen binding protein. A full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

Multispecific antibodies such as bispecific antibodies are antibodies that have binding specificities for at least two different antigens. Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

The antibodies can be multivalent antibodies with two or more antigen binding sites (e.g., tetravalent antibodies), which can be readily produced by recombinant expression of nucleic acid encoding the polypeptide chains of the antibody. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In case there are multiple binding domains in the extracellular antigen binding domain of the present CARs. The various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. In some embodiment, a peptide linker comprises flexible residues (such as glycine and serine) so that the adjacent domains are free to move relative to each other. For example, a glycine-serine doublet can be a suitable peptide linker.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)n, glycine-serine polymers (including, for example, (GS)n, (GSGGS)n (SEQ ID NO: 46), (GGGS)n (SEQ ID NO: 47), and (GGGGS)n (SEQ ID NO: 48), where n is an integer of at least one), glycine-alanine polymers, alanine-serine polymers, and other flexible linkers known in the art. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the CARs provided herein, the disclosure of each of which is incorporated herein by reference.

In some embodiments, the extracellular antigen binding domain provided in the present CARs recognizes an antigen that acts as a cell surface marker on target cells associated with a special disease state. In some embodiments, the antigen is a tumor antigen. Tumors express a number of proteins that can serve as a target antigen for an immune response, particularly T cell mediated immune responses. The antigens targeted by the CAR may be antigens on a single diseased cell or antigens that are expressed on different cells that each contribute to the disease. The antigens targeted by the CAR may be directly or indirectly involved in the diseases.

In some embodiments, the antigen of a target cell is an antigen on the surface of the cancer cell. In some embodiments, the antigen is a tumor-specific antigen, a tumor-associated antigen, or a neoantigen.

In some embodiments, the target cell is a cancer cell, e.g., a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma (MM), neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma (MM), neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is a adrenal cancer. In some embodiments, the cancer is a anal cancer. In some embodiments, the cancer is an appendix cancer. In some embodiments, the cancer is a bile duct cancer. In some embodiments, the cancer is a bladder cancer. In some embodiments, the cancer is a bone cancer. In some embodiments, the cancer is a brain cancer. In some embodiments, the cancer is a breast cancer. In some embodiments, the cancer is a cervical cancer. In some embodiments, the cancer is a colorectal cancer. In some embodiments, the cancer is a esophageal cancer. In some embodiments, the cancer is a gallbladder cancer. In some embodiments, the cancer is a gestational trophoblastic. In some embodiments, the cancer is a head and neck cancer. In some embodiments, the cancer is a Hodgkin lymphoma. In some embodiments, the cancer is an intestinal cancer. In some embodiments, the cancer is a kidney cancer. In some embodiments, the cancer is a leukemia. In some embodiments, the cancer is a liver cancer. In some embodiments, the cancer is a lung cancer. In some embodiments, the cancer is a melanoma. In some embodiments, the cancer is a mesothelioma. In some embodiments, the cancer is a multiple myeloma (MM). In some embodiments, the cancer is a neuroendocrine tumor. In some embodiments, the cancer is a non-Hodgkin lymphoma. In some embodiments, the cancer is an oral cancer. In some embodiments, the cancer is a ovarian cancer. In some embodiments, the cancer is a pancreatic cancer. In some embodiments, the cancer is a prostate cancer. In some embodiments, the cancer is a sinus cancer. In some embodiments, the cancer is a skin cancer. In some embodiments, the cancer is a soft tissue sarcoma spinal cancer. In some embodiments, the cancer is a stomach cancer. In some embodiments, the cancer is a testicular cancer. In some embodiments, the cancer is a throat cancer. In some embodiments, the cancer is a thyroid cancer. In some embodiments, the cancer is a uterine cancer endometrial cancer. In some embodiments, the cancer is a vaginal cancer. In some embodiments, the cancer is a vulvar cancer.

In some embodiments, the adrenal cancer is an adrenocortical carcinoma (ACC), adrenal cortex cancer, pheochromocytoma, or neuroblastoma. In some embodiments, the anal cancer is a squamous cell carcinoma, cloacogenic carcinoma, adenocarcinoma, basal cell carcinoma, or melanoma. In some embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments, the bladder cancer is transitional cell carcinoma (TCC), papillary carcinoma, flat carcinoma, squamous cell carcinoma, adenocarcinoma, small-cell carcinoma, or sarcoma. In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments, the cervical cancer is a squamous cell carcinoma, or adenocarcinoma. In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments, the esophageal cancer is an adenocarcinoma or squamous cell carcinoma. In some embodiments, the gall bladder cancer is an adenocarcinoma, papillary adenocarcinoma, adenosquamous carcinoma, squamous cell carcinoma, small cell carcinoma, or sarcoma. In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments, the intestinal cancer is a small intestine cancer, small bowel cancer, adenocarcinoma, sarcoma, gastrointestinal stromal tumors, carcinoid tumors, or lymphoma. In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML. In some embodiments, the liver cancer is a hepatocellular carcinoma (HCC), fibrolamellar HCC, cholangiocarcinoma, angiosarcoma, or liver metastasis. In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments, the mesothelioma is a pleural mesothelioma, peritoneal mesothelioma, pericardial mesothelioma, or testicular mesothelioma. In some embodiments, the multiple myeloma is an active myeloma or smoldering myeloma. In some embodiments, the neuroendocrine tumor is a gastrointestinal neuroendocrine tumor, pancreatic neuroendocrine tumor, or lung neuroendocrine tumor. In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments, the pancreatic cancer is a pancreatic exocrine gland cancer, pancreatic endocrine gland cancer, or pancreatic adenocarcinoma, islet cell tumor, or neuroendocrine tumor. In some embodiments, the prostate cancer is a prostate adenocarcinoma, prostate sarcoma, transitional cell carcinoma, small cell carcinoma, or neuroendocrine tumor. In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments, the spinal cancer is a spinal metastatic tumor. In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments, the thyroid cancer is a papillary carcinoma, follicular carcinoma, Hürthle cell carcinoma, medullary thyroid carcinoma, or anaplastic carcinoma. In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments, the vaginal cancer is a squamous cell carcinoma, adenocarcinoma, melanoma, or sarcoma. In some embodiments, the vulvar cancer is a squamous cell carcinoma or adenocarcinoma.

Tumor antigens are proteins that are produced by tumor cells that can elicit an immune response, particularly T-cell mediated immune responses. Exemplary tumor antigens include, but not limited to, a glioma-associated antigen, carcinoembryonic antigen (CEA), 0-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, BRCA1, BRCA2, CDK4, CML66, fibronectin, MART-2, p53, Ras, TGF-βRII, or MUC1. In some embodiments, the cancer antigen is CEA. In some embodiments, the cancer antigen is immature laminin receptor. In some embodiments, the cancer antigen is TAG-72. In some embodiments, the cancer antigen is HPV E6. In some embodiments, the cancer antigen is HPV E7. In some embodiments, the cancer antigen is BING-4. In some embodiments, the cancer antigen is calcium-activated chloride channel 2. In some embodiments, the cancer antigen is cyclin-Bi. In some embodiments, the cancer antigen is 9D7. In some embodiments, the cancer antigen is EpCAM. In some embodiments, the cancer antigen is EphA3. In some embodiments, the cancer antigen is Her2/neu. In some embodiments, the cancer antigen is telomerase. In some embodiments, the cancer antigen is mesothelin. In some embodiments, the cancer antigen is SAP-1. In some embodiments, the cancer antigen is surviving. In some embodiments, the cancer antigen is a BAGE family antigen. In some embodiments, the cancer antigen is CAGE family antigen. In some embodiments, the cancer antigen is GAGE family antigen. In some embodiments, the cancer antigen is MAGE family antigen. In some embodiments, the cancer antigen is SAGE family antigen. In some embodiments, the cancer antigen is XAGE family antigen. In some embodiments, the cancer antigen is NY-ESO-1/LAGE-1. In some embodiments, the cancer antigen is PRAME. In some embodiments, the cancer antigen is SSX-2. In some embodiments, the cancer antigen is Melan-A. In some embodiments, the cancer antigen is MART-1. In some embodiments, the cancer antigen is Gp100. In some embodiments, the cancer antigen is pmel17. In some embodiments, the cancer antigen is tyrosinase. In some embodiments, the cancer antigen is TRP-1. In some embodiments, the cancer antigen is TRP-2. In some embodiments, the cancer antigen is P. polypeptide. In some embodiments, the cancer antigen is MC1R. In some embodiments, the cancer antigen is prostate-specific antigen. In some embodiments, the cancer antigen is β-catenin. In some embodiments, the cancer antigen is BRCA1. In some embodiments, the cancer antigen is BRCA2. In some embodiments, the cancer antigen is CDK4. In some embodiments, the cancer antigen is CML66. In some embodiments, the cancer antigen is fibronectin. In some embodiments, the cancer antigen is MART-2. In some embodiments, the cancer antigen is p53. In some embodiments, the cancer antigen is Ras. In some embodiments, the cancer antigen is TGF-βRII. In some embodiments, the cancer antigen is MUC1.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

Additional non-limiting exemplary targets of the CARs provided herein include GPC2, CD276, Delta-like protein ligand 3 (DLL3), NY-ESO-1, melanoma associated antigen 4; survivin protein, synovial sarcoma X breakpoint protein 2, CD3, epidermal growth factor receptor (EGFR), erbb2 tyrosine kinase receptor, HER2, CEA, CD66, CD66e, ROR1, ntrkr1 tyrosine kinase receptor, GPC3, Claudin18.2, mesothelin, glutamate carboxypeptidase II, PMSA, PD-L1, folate receptor alpha, PSCA, Mucin 1, HLA antigen (such as HLA class I antigen A-2 alpha, HLA class I antigen A-11 alpha, and HLA class II antigen), c-Met, hepatocyte growth factor receptor, K-Ras GTPase (KRAS), IL-15 receptor, Kit tyrosine kinase, PDGF receptor beta, RET tyrosine kinase receptor; Raf 1 protein kinase, Raf B protein kinase, thymidylate synthase, topoisomerase II, Brachyury protein, Flt3 tyrosine kinase, VEGF, VEGF receptor (VEGF-1 receptor, VEGF-2 receptor, and VEGF-3 receptor), estrogen receptor, neoantigen, human papillomavirus E6, and heat shock protein.

In some specific embodiments, at least one target antigen of the present CARs is CD19. In other specific embodiments, at least one target antigen of the present CARs is CD20. In yet other specific embodiments, at least one target antigen of the present CARs is CD22. In yet other specific embodiments, at least one target antigen of the present CARs is BCMA. In yet other specific embodiments, at least one target antigen of the present CARs is VEGFR2. In yet other specific embodiments, at least one target antigen of the present CARs is FAP. In yet other specific embodiments, at least one target antigen of the present CARs is EpCam. In yet other specific embodiments, at least one target antigen of the present CARs is GPC3. In yet other specific embodiments, at least one target antigen of the present CARs is Claudin18.2. In yet other specific embodiments, at least one target antigen of the present CARs is CD133. In yet other specific embodiments, at least one target antigen of the present CARs is IL 13Ra. In yet other specific embodiments, at least one target antigen of the present CARs is EGFRIII. In yet other specific embodiments, at least one target antigen of the present CARs is EphA2. In yet other specific embodiments, at least one target antigen of the present CARs is Muc1. In yet other specific embodiments, at least one target antigen of the present CARs is CD70. In yet other specific embodiments, at least one target antigen of the present CARs is CD123. In yet other specific embodiments, at least one target antigen of the present CARs is ROR1. In yet other specific embodiments, at least one target antigen of the present CARs is PSMA. In yet other specific embodiments, at least one target antigen of the present CARs is CD5. In yet other specific embodiments, at least one target antigen of the present CARs is GD2. In yet other specific embodiments, at least one target antigen of the present CARs is GAP. In yet other specific embodiments, at least one target antigen of the present CARs is CD33. In yet other specific embodiments, at least one target antigen of the present CARs is CEA. In yet other specific embodiments, at least one target antigen of the present CARs is PSCA. In yet other specific embodiments, at least one target antigen of the present CARs is Her2. In yet other specific embodiments, at least one target antigen of the present CARs is Mesothelin.

In some embodiments, the CAR provided herein binds to a B cell antigen. In some embodiments, the B cell antigen is a CD1a, CD1b, CD1c, CD1d, CD2, CD5, CD6, CD9, CD11a, CD11b, CD11c, CD17, CD18, CD19, CD20, CD21, CD22, CD23, CD24, CD25, CD26, CD27, CD29, CD30, CD31, CD32a, CD32b, CD35, CD37, CD38, CD39, CD40, CD45, CD45RA, CD45RB, CD45RC, CD45RO, CD46, CD47, CD48, CD49b, CD49c, CD49d, CD50, CD52, CD53, CD54, CD55, CD58, CD60a, CD62L, CD63, CD68, CD69, CD70, CD72, CD73, CD74, CD75, CD75S, CD77, CD79a, CD79b, CD80, CD81, CD82, CD83, CD84, CD85E, CD85I, CD85J, CD86, CD92, CD95, CD97, CD98, CD99, CD100, CD102, CD108, CD119, CD120a, CD120b, CD121b, CD122, CD124, CD125, CD126, CD130, CD132, CD137, CD138, CD139, CD147, CD148, CD150, CD152, CD162, CD164, CD166, CD167a, CD170, CD171, CD175, CD175s, CD180, CD184, CD185, CD192, CD196, CD197, CD200, CD205, CD201a, CDw210b, CD212, CD213a1, CD213a2, CD 215, CD217, CD218a, CD218b, CD220, CD221, CD222, CD224, CD225, CD226, CD227, CD229, CD230, CD232, CD252, CD252, CD254, CD255, CD256, CD257 CD258, CD259, CD260, CD261, CD262, CD263, CD264, CD267, CD268, CD269, CD270, CD272, CD274, CD275, CD277, CD279, CD283, CD289, CD290, CD295, CD298, CD300, CD300c, CD305, CD306, CD307a, CD307b, CD307c, CD307d, CD307e, CD314, CD215, CD316, CD317, CD319, CD321, CD327, CD328, CD329, CD338, CD351, CD352, CD353, CD354, CD355, CD356, CD357, CD358, CD360, CD361, CD362, or CD363 antigen. In some embodiments, the B cell antigen is a CD1a antigen. In some embodiments, the B cell antigen is a CD1b antigen. In some embodiments, the B cell antigen is a CD1c antigen. In some embodiments, the B cell antigen is a CD1d antigen. In some embodiments, the B cell antigen is a CD2 antigen. In some embodiments, the B cell antigen is a CD5 antigen. In some embodiments, the B cell antigen is a CD6 antigen. In some embodiments, the B cell antigen is a CD9 antigen. In some embodiments, the B cell antigen is a CD11a antigen. In some embodiments, the B cell antigen is a CD11b antigen. In some embodiments, the B cell antigen is a CD11c antigen. In some embodiments, the B cell antigen is a CD17 antigen. In some embodiments, the B cell antigen is a CD18 antigen. In some embodiments, the B cell antigen is a CD19 antigen. In some embodiments, the B cell antigen is a CD20 antigen. In some embodiments, the B cell antigen is a CD21 antigen. In some embodiments, the B cell antigen is a CD22 antigen. In some embodiments, the B cell antigen is a CD23 antigen. In some embodiments, the B cell antigen is a CD24 antigen. In some embodiments, the B cell antigen is a CD25 antigen. In some embodiments, the B cell antigen is a CD26 antigen. In some embodiments, the B cell antigen is a CD27 antigen. In some embodiments, the B cell antigen is a CD29 antigen. In some embodiments, the B cell antigen is a CD30 antigen. In some embodiments, the B cell antigen is a CD31 antigen. In some embodiments, the B cell antigen is a CD32a antigen. In some embodiments, the B cell antigen is a CD32b antigen. In some embodiments, the B cell antigen is a CD35 antigen. In some embodiments, the B cell antigen is a CD37 antigen. In some embodiments, the B cell antigen is a CD38 antigen. In some embodiments, the B cell antigen is a CD39 antigen. In some embodiments, the B cell antigen is a CD40 antigen. In some embodiments, the B cell antigen is a CD45 antigen. In some embodiments, the B cell antigen is a CD45RA antigen. In some embodiments, the B cell antigen is a CD45RB antigen. In some embodiments, the B cell antigen is a CD45RC antigen. In some embodiments, the B cell antigen is a CD45RO antigen. In some embodiments, the B cell antigen is a CD46 antigen. In some embodiments, the B cell antigen is a CD47 antigen. In some embodiments, the B cell antigen is a CD48 antigen. In some embodiments, the B cell antigen is a CD49b antigen. In some embodiments, the B cell antigen is a CD49c antigen. In some embodiments, the B cell antigen is a CD49d antigen. In some embodiments, the B cell antigen is a CD50 antigen. In some embodiments, the B cell antigen is a CD52 antigen. In some embodiments, the B cell antigen is a CD53 antigen. In some embodiments, the B cell antigen is a CD54 antigen. In some embodiments, the B cell antigen is a CD55 antigen. In some embodiments, the B cell antigen is a CD58 antigen. In some embodiments, the B cell antigen is a CD60a antigen. In some embodiments, the B cell antigen is a CD62L antigen. In some embodiments, the B cell antigen is a CD63 antigen. In some embodiments, the B cell antigen is a CD68 antigen. In some embodiments, the B cell antigen is a CD69 antigen. In some embodiments, the B cell antigen is a CD70 antigen. In some embodiments, the B cell antigen is a CD72 antigen. In some embodiments, the B cell antigen is a CD73 antigen. In some embodiments, the B cell antigen is a CD74 antigen. In some embodiments, the B cell antigen is a CD75 antigen. In some embodiments, the B cell antigen is a CD75S antigen. In some embodiments, the B cell antigen is a CD77 antigen. In some embodiments, the B cell antigen is a CD79a antigen. In some embodiments, the B cell antigen is a CD79b antigen. In some embodiments, the B cell antigen is a CD80 antigen. In some embodiments, the B cell antigen is a CD81 antigen. In some embodiments, the B cell antigen is a CD82 antigen. In some embodiments, the B cell antigen is a CD83 antigen. In some embodiments, the B cell antigen is a CD84 antigen. In some embodiments, the B cell antigen is a CD85E antigen. In some embodiments, the B cell antigen is a CD85I antigen. In some embodiments, the B cell antigen is a CD85J antigen. In some embodiments, the B cell antigen is a CD86 antigen. In some embodiments, the B cell antigen is a CD92 antigen. In some embodiments, the B cell antigen is a CD95 antigen. In some embodiments, the B cell antigen is a CD97 antigen. In some embodiments, the B cell antigen is a CD98 antigen. In some embodiments, the B cell antigen is a CD99 antigen. In some embodiments, the B cell antigen is a CD100 antigen. In some embodiments, the B cell antigen is a CD102 antigen. In some embodiments, the B cell antigen is a CD108 antigen. In some embodiments, the B cell antigen is a CD 119 antigen. In some embodiments, the B cell antigen is a CD120a antigen. In some embodiments, the B cell antigen is a CD120b antigen. In some embodiments, the B cell antigen is a CD121b antigen. In some embodiments, the B cell antigen is a CD122 antigen. In some embodiments, the B cell antigen is a CD124 antigen. In some embodiments, the B cell antigen is a CD125 antigen. In some embodiments, the B cell antigen is a CD126 antigen. In some embodiments, the B cell antigen is a CD130 antigen. In some embodiments, the B cell antigen is a CD132 antigen. In some embodiments, the B cell antigen is a CD137 antigen. In some embodiments, the B cell antigen is a CD138 antigen. In some embodiments, the B cell antigen is a CD139 antigen. In some embodiments, the B cell antigen is a CD147 antigen. In some embodiments, the B cell antigen is a CD148 antigen. In some embodiments, the B cell antigen is a CD150 antigen. In some embodiments, the B cell antigen is a CD152 antigen. In some embodiments, the B cell antigen is a CD162 antigen. In some embodiments, the B cell antigen is a CD164 antigen. In some embodiments, the B cell antigen is a CD166 antigen. In some embodiments, the B cell antigen is a CD167a antigen. In some embodiments, the B cell antigen is a CD170 antigen. In some embodiments, the B cell antigen is a CD171 antigen. In some embodiments, the B cell antigen is a CD175 antigen. In some embodiments, the B cell antigen is a CD175s antigen. In some embodiments, the B cell antigen is a CD180 antigen. In some embodiments, the B cell antigen is a CD184 antigen. In some embodiments, the B cell antigen is a CD185 antigen. In some embodiments, the B cell antigen is a CD192 antigen. In some embodiments, the B cell antigen is a CD196 antigen. In some embodiments, the B cell antigen is a CD197 antigen. In some embodiments, the B cell antigen is a CD200 antigen. In some embodiments, the B cell antigen is a CD205 antigen. In some embodiments, the B cell antigen is a CD201a antigen. In some embodiments, the B cell antigen is a CDw210b antigen. In some embodiments, the B cell antigen is a CD212 antigen. In some embodiments, the B cell antigen is a CD213a1 antigen. In some embodiments, the B cell antigen is a CD213a2 antigen. In some embodiments, the B cell antigen is a CD 215 antigen. In some embodiments, the B cell antigen is a CD217 antigen. In some embodiments, the B cell antigen is a CD218a antigen. In some embodiments, the B cell antigen is a CD218b antigen. In some embodiments, the B cell antigen is a CD220 antigen. In some embodiments, the B cell antigen is a CD221 antigen. In some embodiments, the B cell antigen is a CD222 antigen. In some embodiments, the B cell antigen is a CD224 antigen. In some embodiments, the B cell antigen is a CD225 antigen. In some embodiments, the B cell antigen is a CD226 antigen. In some embodiments, the B cell antigen is a CD227 antigen. In some embodiments, the B cell antigen is a CD229 antigen. In some embodiments, the B cell antigen is a CD230 antigen. In some embodiments, the B cell antigen is a CD232 antigen. In some embodiments, the B cell antigen is a CD252 antigen. In some embodiments, the B cell antigen is a CD252 antigen. In some embodiments, the B cell antigen is a CD254 antigen. In some embodiments, the B cell antigen is a CD255 antigen. In some embodiments, the B cell antigen is a CD256 antigen. In some embodiments, the B cell antigen is a CD257 CD258 antigen. In some embodiments, the B cell antigen is a CD259 antigen. In some embodiments, the B cell antigen is a CD260 antigen. In some embodiments, the B cell antigen is a CD261 antigen. In some embodiments, the B cell antigen is a CD262 antigen. In some embodiments, the B cell antigen is a CD263 antigen. In some embodiments, the B cell antigen is a CD264 antigen. In some embodiments, the B cell antigen is a CD267 antigen. In some embodiments, the B cell antigen is a CD268 antigen. In some embodiments, the B cell antigen is a CD269 antigen. In some embodiments, the B cell antigen is a CD270 antigen. In some embodiments, the B cell antigen is a CD272 antigen. In some embodiments, the B cell antigen is a CD274 antigen. In some embodiments, the B cell antigen is a CD275 antigen. In some embodiments, the B cell antigen is a CD277 antigen. In some embodiments, the B cell antigen is a CD279 antigen. In some embodiments, the B cell antigen is a CD283 antigen. In some embodiments, the B cell antigen is a CD289 antigen. In some embodiments, the B cell antigen is a CD290 antigen. In some embodiments, the B cell antigen is a CD295 antigen. In some embodiments, the B cell antigen is a CD298 antigen. In some embodiments, the B cell antigen is a CD300 antigen. In some embodiments, the B cell antigen is a CD300c antigen. In some embodiments, the B cell antigen is a CD305 antigen. In some embodiments, the B cell antigen is a CD306 antigen. In some embodiments, the B cell antigen is a CD307a antigen. In some embodiments, the B cell antigen is a CD307b antigen. In some embodiments, the B cell antigen is a CD307c antigen. In some embodiments, the B cell antigen is a CD307d antigen. In some embodiments, the B cell antigen is a CD307e antigen. In some embodiments, the B cell antigen is a CD314 antigen. In some embodiments, the B cell antigen is a CD215 antigen. In some embodiments, the B cell antigen is a CD316 antigen. In some embodiments, the B cell antigen is a CD317 antigen. In some embodiments, the B cell antigen is a CD319 antigen. In some embodiments, the B cell antigen is a CD321 antigen. In some embodiments, the B cell antigen is a CD327 antigen. In some embodiments, the B cell antigen is a CD328 antigen. In some embodiments, the B cell antigen is a CD329 antigen. In some embodiments, the B cell antigen is a CD338 antigen. In some embodiments, the B cell antigen is a CD351 antigen. In some embodiments, the B cell antigen is a CD352 antigen. In some embodiments, the B cell antigen is a CD353 antigen. In some embodiments, the B cell antigen is a CD354 antigen. In some embodiments, the B cell antigen is a CD355 antigen. In some embodiments, the B cell antigen is a CD356 antigen. In some embodiments, the B cell antigen is a CD357 antigen. In some embodiments, the B cell antigen is a CD358 antigen. In some embodiments, the B cell antigen is a CD360 antigen. In some embodiments, the B cell antigen is a CD361 antigen. In some embodiments, the B cell antigen is a CD362 antigen. In some embodiments, the B cell antigen is a CD363 antigen.

In one embodiment, target of the present CAR is a pathogen. In certain embodiments, the target cell is a cell comprising a pathogen.

In some embodiments, the pathogen causes an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection. In some embodiments, the infectious disease is Acute Flaccid Myelitis (AFM). In some embodiments, the infectious disease is Anaplasmosis. In some embodiments, the infectious disease is Anthrax. In some embodiments, the infectious disease is Babesiosis. In some embodiments, the infectious disease is Botulism. In some embodiments, the infectious disease is Brucellosis. In some embodiments, the infectious disease is Campylobacteriosis. In some embodiments, the infectious disease is Carbapenem-resistant Infection. In some embodiments, the infectious disease is Chancroid. In some embodiments, the infectious disease is Chikungunya Virus Infection. In some embodiments, the infectious disease is Chlamydia. In some embodiments, the infectious disease is Ciguatera. In some embodiments, the infectious disease is Difficile Infection. In some embodiments, the infectious disease is Perfringens. In some embodiments, the infectious disease is Coccidioidomycosis fungal infection. In some embodiments, the infectious disease is coronavirus. In some embodiments, the infectious disease is Covid-19 (SARS-CoV-2). In some embodiments, the infectious disease is Creutzfeldt-Jacob Disease/transmissible spongiform encephalopathy. In some embodiments, the infectious disease is Cryptosporidiosis (Crypto). In some embodiments, the infectious disease is Cyclosporiasis. In some embodiments, the infectious disease is Dengue 1, 2, 3 or 4. In some embodiments, the infectious disease is Diphtheria. In some embodiments, the infectious disease is E. coli infection/Shiga toxin-producing (STEC). In some embodiments, the infectious disease is Eastern Equine Encephalitis. In some embodiments, the infectious disease is Hemorrhagic Fever (Ebola). In some embodiments, the infectious disease is Ehrlichiosis. In some embodiments, the infectious disease is Encephalitis. In some embodiments, the infectious disease is Arboviral or parainfectious. In some embodiments, the infectious disease is Non-Polio Enterovirus. In some embodiments, the infectious disease is D68 Enteroviru (EV-D68). In some embodiments, the infectious disease is Giardiasis. In some embodiments, the infectious disease is Glanders. In some embodiments, the infectious disease is Gonococcal Infection. In some embodiments, the infectious disease is Granuloma inguinale. In some embodiments, the infectious disease is Haemophilus influenza disease Type B (Hib or H-flu). In some embodiments, the infectious disease is Hantavirus Pulmonary Syndrome (HPS). In some embodiments, the infectious disease is Hemolytic Uremic Syndrome (HUS). In some embodiments, the infectious disease is Hepatitis A (Hep A). In some embodiments, the infectious disease is Hepatitis B (Hep B). In some embodiments, the infectious disease is Hepatitis C (Hep C). In some embodiments, the infectious disease is Hepatitis D (Hep D). In some embodiments, the infectious disease is Hepatitis E (Hep E). In some embodiments, the infectious disease is Herpes. In some embodiments, the infectious disease is Herpes Zoster (Shingles). In some embodiments, the infectious disease is Histoplasmosis infection. In some embodiments, the infectious disease is Human Immunodeficiency Virus/AIDS (HIV/AIDS). In some embodiments, the infectious disease is Human Papillomavirus (HPV). In some embodiments, the infectious disease is influenza (Flu). In some embodiments, the infectious disease is Legionellosis (Legionnaires Disease). In some embodiments, the infectious disease is Leprosy (Hansens Disease). In some embodiments, the infectious disease is Leptospirosis. In some embodiments, the infectious disease is Listeriosis (Listeria). In some embodiments, the infectious disease is Lyme Disease. In some embodiments, the infectious disease is Lymphogranuloma venereum infection (LGV). In some embodiments, the infectious disease is Malaria. In some embodiments, the infectious disease is Measles. In some embodiments, the infectious disease is Melioidosis. In some embodiments, the infectious disease is Meningitis (Viral). In some embodiments, the infectious disease is Meningococcal Disease (Meningitis (Bacterial)). In some embodiments, the infectious disease is Middle East Respiratory Syndrome Coronavirus (MERS-CoV). In some embodiments, the infectious disease is Mumps. In some embodiments, the infectious disease is Norovirus. In some embodiments, the infectious disease is Pediculosis. In some embodiments, the infectious disease is Pelvic Inflammatory Disease (PID). In some embodiments, the infectious disease is Pertussis (Whooping Cough). In some embodiments, the infectious disease is Plague (Bubonic. In some embodiments, the infectious disease is Septicemic. In some embodiments, the infectious disease is Pneumonic). In some embodiments, the infectious disease is Pneumococcal Disease (Pneumonia). In some embodiments, the infectious disease is Poliomyelitis (Polio). In some embodiments, the infectious disease is Powassan. In some embodiments, the infectious disease is Psittacosis. In some embodiments, the infectious disease is Pthiriasis. In some embodiments, the infectious disease is Pustular Rash diseases (Small pox. In some embodiments, the infectious disease is monkeypox. In some embodiments, the infectious disease is cowpox). In some embodiments, the infectious disease is Q-Fever. In some embodiments, the infectious disease is Rabies. In some embodiments, the infectious disease is Rickettsiosis (Rocky Mountain Spotted Fever). In some embodiments, the infectious disease is Rubella (German Measles). In some embodiments, the infectious disease is Salmonellosis gastroenteritis (Salmonella). In some embodiments, the infectious disease is Scabies. In some embodiments, the infectious disease is Scombroid. In some embodiments, the infectious disease is Sepsis. In some embodiments, the infectious disease is Severe Acute Respiratory Syndrome (SARS). In some embodiments, the infectious disease is Shigellosis gastroenteritis (Shigella). In some embodiments, the infectious disease is Smallpox. In some embodiments, the infectious disease is Staphyloccal Infection Methicillin-resistant (MRSA). In some embodiments, the infectious disease is Staphylococcal Food Poisoning Enterotoxin B Poisoning (Staph Food Poisoning). In some embodiments, the infectious disease is Saphylococcal Infection Vancomycin Intermediate (VISA). In some embodiments, the infectious disease is Staphylococcal Infection Vancomycin Resistant (VRSA). In some embodiments, the infectious disease is Streptococcal Disease Group A (invasive) (Strep A (invasive). In some embodiments, the infectious disease is Streptococcal Disease. In some embodiments, the infectious disease is Group B (Strep-B). In some embodiments, the infectious disease is Streptococcal Toxic-Shock Syndrome STSS Toxic Shock. In some embodiments, the infectious disease is Syphilis (primary. In some embodiments, the infectious disease is secondary. In some embodiments, the infectious disease is early latent. In some embodiments, the infectious disease is late latent. In some embodiments, the infectious disease is congenital). In some embodiments, the infectious disease is Tetanus Infection. In some embodiments, the infectious disease is Trichomoniasis. In some embodiments, the infectious disease is Trichonosis Infection. In some embodiments, the infectious disease is Tuberculosis (TB). In some embodiments, the infectious disease is Tuberculosis Latent (LTBI). In some embodiments, the infectious disease is Tularemia. In some embodiments, the infectious disease is Typhoid Fever Group D. In some embodiments, the infectious disease is Vaginosis. In some embodiments, the infectious disease is Varicella (Chickenpox), Vibrio cholerae (Cholera). In some embodiments, the infectious disease is Vibriosis (Vibrio). In some embodiments, the infectious disease is Ebola Virus Hemorrhagic Fever. In some embodiments, the infectious disease is Lasa Virus Hemorrhagic Fever. In some embodiments, the infectious disease is Marburg Virus Hemorrhagic Fever. In some embodiments, the infectious disease is West Nile Virus. In some embodiments, the infectious disease is Yellow Fever. In some embodiments, the infectious disease is Yersenia. In some embodiments, the infectious disease is and Zika Virus Infection.

In some embodiments, the pathogen is a bacteria. In some embodiments, the bacteria is a bacteria of a Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio or Yersinia genus. In some embodiments, the bacteria is a bacteria of the Bacillus genus. In some embodiments, the bacteria is a bacteria of the Bartonella genus. In some embodiments, the bacteria is a bacteria of the Bordetella genus. In some embodiments, the bacteria is a bacteria of the Borrelia genus. In some embodiments, the bacteria is a bacteria of the Brucella genus. In some embodiments, the bacteria is a bacteria of the Campylobacter genus. In some embodiments, the bacteria is a bacteria of the Chlamydia genus. In some embodiments, the bacteria is a bacteria of the Chlamydophila genus. In some embodiments, the bacteria is a bacteria of the clostridium genus. In some embodiments, the bacteria is a bacteria of the Corynebacterium genus. In some embodiments, the bacteria is a bacteria of the Enterococcus genus. In some embodiments, the bacteria is a bacteria of the Escherichia genus. In some embodiments, the bacteria is a bacteria of the Francisella genus. In some embodiments, the bacteria is a bacteria of the Haemophilus genus. In some embodiments, the bacteria is a bacteria of the Helicobacter genus. In some embodiments, the bacteria is a bacteria of the Legionella genus. In some embodiments, the bacteria is a bacteria of the leptospira genus. In some embodiments, the bacteria is a bacteria of the Listeria genus. In some embodiments, the bacteria is a bacteria of the Mycobacterium genus. In some embodiments, the bacteria is a bacteria of the Mycoplasma genus. In some embodiments, the bacteria is a bacteria of the Neisseria genus. In some embodiments, the bacteria is a bacteria of the Pseudomonas genus. In some embodiments, the bacteria is a bacteria of the Rickettsia genus. In some embodiments, the bacteria is a bacteria of the Salmonella genus. In some embodiments, the bacteria is a bacteria of the Shigella genus. In some embodiments, the bacteria is a bacteria of the Staphylococcus genus. In some embodiments, the bacteria is a bacteria of the Streptococcus genus. In some embodiments, the bacteria is a bacteria of the Treponema genus. In some embodiments, the bacteria is a bacteria of the Ureaplasma genus. In some embodiments, the bacteria is a bacteria of the Vibrio genus. In some embodiments, the bacteria is a bacteria of the Yersinia genus.

In some embodiments, the pathogen is a parasite. In some embodiments, the parasite is a protozoa, helminth, or ectoparasite. In some embodiments, the protozoa is an Entamoeba, Giardia, Leishmania, Balantidium, Plasmodium, or Cryptosporidium. In some embodiments, the helminth is a trematode, cestode, acanthocephalan, or round worm. In some embodiments, the ectoparasite is a arthropod.

In some embodiments, the pathogen is a virus. In some embodiments, the virus is a virus of the adenoviridae, arenaviridae, astroviridae, bunyaviridae, caliciviridae, coronaviridae, filoviridae, flaviviridae, hepadnaviridae, hepeviridae, orthomyxoviridae, papillomaviridae, paramyxoviridae, parvoviridae, picomaviridae, polyomaviridae, poxviridae, reoviridae, retroviridae, rhabdoviridae, or togaviridae family. In some embodiments family. In some embodiments, the virus is a virus of the virus is a virus of the adenoviridae family. In some embodiments, the virus is a virus of the arenaviridae family. In some embodiments, the virus is a virus of the astroviridae family. In some embodiments, the virus is a virus of the bunyaviridae family. In some embodiments, the virus is a virus of the caliciviridae family. In some embodiments, the virus is a virus of the coronaviridae family. In some embodiments, the virus is a virus of the filoviridae family. In some embodiments, the virus is a virus of the flaviviridae family. In some embodiments, the virus is a virus of the hepadnaviridae family. In some embodiments, the virus is a virus of the hepeviridae family. In some embodiments, the virus is a virus of the orthomyxoviridae family. In some embodiments, the virus is a virus of the papillomaviridae family. In some embodiments, the virus is a virus of the paramyxoviridae family. In some embodiments, the virus is a virus of the parvoviridae family. In some embodiments, the virus is a virus of the picomaviridae family. In some embodiments, the virus is a virus of the polyomaviridae family. In some embodiments, the virus is a virus of the poxviridae family. In some embodiments, the virus is a virus of the reoviridae family. In some embodiments, the virus is a virus of the retroviridae family. In some embodiments, the virus is a virus of the rhabdoviridae family. In some embodiments, the virus is a virus of the togaviridae family.

In some embodiments, the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus. In some embodiments, the virus is an adenovirus. In some embodiments, the virus is a coronavirus. In some embodiments, the coronavirus virus is Covid-19 (SARS-CoV-2). In some embodiments, the virus is a coxsackievirus. In some embodiments, the virus is a Epstein-Barr virus. In some embodiments, the virus is a hepatitis A virus. In some embodiments, the virus is a hepatitis B virus. In some embodiments, the virus is a hepatitis C virus. In some embodiments, the virus is a herpes simplex virus type 2. In some embodiments, the virus is a cytomegalovirus. In some embodiments, the virus is a human herpes virus type 8. In some embodiments, the virus is a human immunodeficiency virus. In some embodiments, the virus is an influenza virus. In some embodiments, the virus is a measles virus. In some embodiments, the virus is a mumps virus. In some embodiments, the virus is a human papillomavirus. In some embodiments, the virus is a parainfluenza virus. In some embodiments, the virus is a poliovirus. In some embodiments, the virus is a rabies virus. In some embodiments, the virus is a respiratory syncytial virus. In some embodiments, the virus is a rubella virus. In some embodiments, the virus is a varicella-zoster virus.

### Transmembrane Domain

The CARs of the present disclosure comprise a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, preferably a eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the CAR provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some specific embodiments, the transmembrane domain is derived from CD8α. In other specific embodiments, the transmembrane domain is derived from CD28α.

### Intracellular Signaling Domain

The intracellular signaling domain in the CARs provided herein is responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3z, FcR gamma (FCER1G), FcR beta (Fc Epsilon Rib), CD3 gamma, CD3 delta, CD3 epsilon, CD5, CD22, CD79a, CD79b, and CD66d.

### Co-Stimulatory Signaling Domain

In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. Many immune effector cells require co-stimulation, in addition to stimulation of an antigen-specific signal, to promote cell proliferation, differentiation and survival, as well as to activate effector functions of the cell.

The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3z) and one or more co-stimulatory signaling domains. In some embodiments, the one or more co-stimulatory signaling domains and the primary intracellular signaling domain (such as cytoplasmic signaling domain of CD3z) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3z). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24NISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27nTNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, CD3, lymphocyte function-associated antigen-1 (LFA-1), CD2, CD7, LIGHT, NKG2C, B7-H3 and ligands that specially bind to CD83.

In some embodiments, the co-stimulatory signaling domains are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### Signal Peptide

In certain embodiments, the CARs provided herein may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α, GM-CSF receptor α, and IgG1 heavy chain.

### Hinge Region

In some embodiments, the CARs provided herein comprise a hinge domain that is located between the extracellular antigen binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

Hinge domains of antibodies, such as an IgG, IgA, IgM, IgE, or IgD antibodies, are also compatible for use in the pH-dependent chimeric receptor systems described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (GxS)n linker, wherein x and n, independently can be an integer between 3 and 12, including 3, 4, 5, 6, 7, 8, 9, 10, 11, 12, or more.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor.

In some specific embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α. In other embodiments, the hinge domain is derived from CD28α. In some embodiments, the hinge domain is a portion of the hinge domain of CD28α, e.g., a fragment containing at least 15 (e.g., 20, 25, 30, 35, or 40) consecutive amino acids of the hinge domain of CD28α.

### Exemplary CARs

Any CARs can be used in the present disclosure, including but not limited to Kymria™ (tisagenlecleucel), Yescart™ (axicabtagene ciloleucel), ALEXIS AIDT-2 EOC (Kiromic Biopharma Inc), CIK-CAR.PSMA (Formula Pharmaceuticals Inc), ADI-002 (Adicet Bio Inc), TSC-200 (TScan Therapeutics Inc), TSC-100 (TScan Therapeutics Inc), RB-H21 (Refuge Biotechnologies Inc), ADP-A2AFP (Adaptimmune Therapeutics plc), CT-0729 (Carisma Therapeutics), CT-1119 (Carisma Therapeutics), CCT-301-59 (EXUMA Biotech Corp), BOXR-889 (Unum Therapeutics Inc), meso-CAR-T+PD-78 (MirImmune LLC), MAGE-A10C796T (Adaptimmune Therapeutics plc), NKG2D-DARIC T-cells (Bluebird Bio Inc), AGENt-NY-ESO-1× (AgenTus Therapeutics Inc), MB-105 (City of Hope Medical Center), P-MUC1C-101 (Poseida Therapeutics Inc), TT-16 (Baylor College of Medicine), ACTR-087 (Unum Therapeutics Inc), EGFR-806 (Seattle Children's Hospital), TAC01-ROR1 (Triumvira Immunologics Inc), CCT-301-38 (EXUMA Biotech Corp), MB-103 (Mustang Bio Inc), TAB-28z (OncoTab Inc), ET-1402 (Eureka Therapeutics Inc), ITI-1000 (Duke University), CIDeCAR (Bellicum Pharmaceuticals Inc), MT-201 (Myeloid Therapeutics Inc), CT-0508 (Carisma Therapeutics), ADP-A2M4 (Adaptimmune Therapeutics plc), AMG-119 (Amgen Inc), iCasp9M28z T cells (Memorial Sloan-Kettering Cancer Center), P-PSMA-101 (Poseida Therapeutics Inc), ACE-1702 (Acepodia Inc), AIC-100 (Cornell University), PRGN-3005 (Precigen Inc), UniCAR-T-PSMA (GEMoaB Monoclonals GmbH), IMA-204 (MD Anderson Cancer Center), DSG3-CAART (University of Pennsylvania), LXF-821 (University of Pennsylvania), MOv19-BBz CAR T cells (University of Pennsylvania), Tn MUC-1 CAR-T (University of Pennsylvania), huMesoCART (University of Pennsylvania), IMA-203 (Immatics NV), IMA-202 (Immatics NV), XYP-317 (Xyphos Inc), CYAD-101 (Celyad Oncology), huMNC2-CAR44 T cells (Minerva Biotechnologies Corp), HER2Bi-armed ATC (Roger Williams Medical Center), and CYAD-200 series (Celdara Medical LLC), Lisocabtagene Maraleucel (liso-cel), FT596 (Fate Therapeutics), KTE-C19 (Kite Pharma Inc), KTE-X19 (Kite Pharma Inc), KITE-718 (Kite Pharma Inc), KITE-439 (Kite Pharma Inc), JCAR-024 (Fred Hutchinson Cancer Research Center), JCAR-023 (Juno Therapeutics Inc), BPX-201 (Bellicum Pharmaceuticals Inc), BPX-601 (Bellicum Pharmaceuticals Inc), BPX-603 (Bellicum Pharmaceuticals Inc), MCY-M11 (MaxCyte Inc), KUR-503 (Baylor College of Medicine), ICS-200 (University of Alabama at Birmingham), ADP-A2M4CD8 (Adaptimmune Therapeutics plc), GLYCAR (Baylor College of Medicine).

In certain embodiments, the CAR provided herein comprises amino acid sequences with certain percent identity relative to any one of the exemplary CARs described above and in Section 8 below. In some embodiments, provided herein is a CAR comprising or consisting of an extracellular domain having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of the CARs described above and in Section 8 below.

In some embodiments, amino acid sequence modification(s) of the CARs described herein are contemplated. For example, it may be desirable to optimize the binding affinity and/or other biological properties of the extracellular domain, including but not limited to specificity, thermostability, expression level, effector functions, glycosylation, reduced immunogenicity, or solubility. Thus, in addition to the extracellular domain described herein, it is contemplated that variants of the domains described herein can be prepared. For example, scFv variants can be prepared by introducing appropriate nucleotide changes into the encoding DNA, and/or by synthesis of the desired antibody or polypeptide. Those skilled in the art who appreciate that amino acid changes may alter post-translational processes of the antibody.

Variations may be a substitution, deletion, or insertion of one or more codons encoding the polypeptide that results in a change in the amino acid sequence as compared with the original antibody or polypeptide. Sites of interest for substitutional mutagenesis include the CDRs and FRs.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental polypeptides.

The polypeptides generated by conservative amino acid substitutions are included in the present disclosure. Conservative (e.g., within an amino acid group with similar properties and/or side chains) substitutions may be made, so as to maintain or not significantly change the properties.

One type of substitutional variant involves substituting one or more hypervariable region residues of a parent antibody (e.g., a humanized or human antibody) or fragment thereof in the extracellular antigen binding domain of the present CARs. Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see. e.g., Chowdhury, Methods Mol. Biol. 207:179-196 (2008)), and/or SDRs (a-CDRs), with the resulting variant antibody or fragment thereof being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, 244:1081-1085 (1989). Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

The variations can be made using methods known in the art such as oligonucleotide-mediated (site-directed) mutagenesis, alanine scanning, and PCR mutagenesis. Site-directed mutagenesis (see. e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the antibody variant DNA.

Also included in the present disclosure are immune effector cells comprising two or more kinds of functional exogenous receptors, such dual CARs that binds two different targets.

### 7.2.3. Immune Effector Cells

“Immune effector cells” are immune cells that can perform immune effector functions. In some embodiments, the immune effector cells express at least FcγRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils.

In some embodiments, the immune effector cells are T cells. In some embodiments, the T cells are CD4+/CD8−, CD4−/CD8+, CD4+/CD8+, CD4−/CD8−, or combinations thereof. In some embodiments, the T cells produce IL-2, TFN, and/or TNF upon expressing the CAR and binding to the target cells. In some embodiments, the CD8+ T cells lyse antigen-specific target cells upon expressing the CAR and binding to the target cells.

In some embodiments, the immune effector cells are NK cells. In other embodiments, the immune effector cells can be established cell lines, for example, NK-92 cells.

In some embodiments, the immune effector cells are differentiated from a stem cell, such as a hematopoietic stem cell, a pluripotent stem cell, an iPS, or an embryonic stem cell.

The engineered immune effector cells are prepared by introducing the polypeptide provided herein into the immune effector cells, such as T cells. In some embodiments, the polypeptide is introduced to the immune effector cells by transfecting any one of the isolated nucleic acids or any one of the vectors described above.

Methods of introducing vectors or isolated nucleic acids into a mammalian cell are known in the art. The vectors described can be transferred into an immune effector cell by physical, chemical, or biological methods.

Physical methods for introducing the vector into an immune effector cell include calcium phosphate precipitation, lipofection, particle bombardment, microinjection, electroporation, and the like. Methods for producing cells comprising vectors and/or exogenous nucleic acids are well-known in the art. See, e.g., Sambrook et al. (2001) Molecular Cloning: A Laboratory Manual, Cold Spring Harbor Laboratory, New York. In some embodiments, the vector is introduced into the cell by electroporation.

Biological methods for introducing the vector into an immune effector cell include the use of DNA and RNA vectors. Viral vectors have become the most widely used method for inserting genes into mammalian, e.g., human cells.

Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the polypeptides described herein may be prepared by a conventional method (e.g., in vitro transcription) and then introduced into the immune effector cells via known methods such as mRNA electroporation. See, e.g., Rabinovich et al., Human Gene Therapy 17:1027-1035 (2006).

In some embodiments, the transduced or transfected immune effector cell is propagated ex vivo after introduction of the vector or isolated nucleic acid. In some embodiments, the transduced or transfected immune effector cell is cultured to propagate for at least about any of 1 day, 2 days, 3 days, 4 days, 5 days, 6 days, 7 days, 10 days, 12 days, or 14 days. In some embodiments, the transduced or transfected immune effector cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially.

Other methods to confirm the presence of the nucleic acid encoding the polypeptide in the engineered immune effector cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

### 7.2.4. Sources of T Cells

In some embodiments, prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Initial activation steps in the absence of calcium may lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, in some embodiments, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immuno adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/mL is used. In one embodiment, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and cell expansion. Further, use of high cell concentrations may allow more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. In some embodiments, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/mL. In some embodiments, the concentration used can be from about 1×10⁵/mL to 1×10⁶/mL, and any integer value in between.

In some embodiments, the cells may be incubated on a rotator for varying lengths of time at varying speeds at either 2-10° C., or at room temperature.

T cells for stimulation can also be frozen after a washing step. Without being bound by theory, the freeze and subsequent thaw step may provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% dextran 40 and 5% dextrose, 20% human serum albumin and 7.5% DMSO, or 31.25% plasmalyte-A, 31.25% dextrose 5%, 0.45% NaCl, 10% dextran 40 and 5% dextrose, 20% human serum albumin, and 7.5% DMSO or other suitable cell freezing media containing for example, Hespan and PlasmaLyte A. The cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment, a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815 (1991); Henderson et al., Immun 73:316-321 (1991); Bierer et al., Curr. Opin. Immun. 5:763-773 (1993)). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### 7.2.5. Activation and Expansion of T Cells

In some embodiments, prior to or after genetic modification of the T cells with the CARs described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD3 antibody include UCHT1, OKT3, HIT3a (BioLegend, San Diego, US) can be used as can other methods commonly known in the art (Graves J, et al., J. Immunol. 146:2102 (1991); Li B, et al., Immunology 116:487 (2005); Rivollier A, et al., Blood 104:4029 (2004)). Examples of an anti-CD28 antibody include 9.3, B-T3, XR-CD28 (Diaclone, Besancon, France) can be used as can other methods commonly known in the art (Berg et al., Transplant Proc. 30(8):3975-3977 (1998); Haanen et al., J. Exp. Med. 190(9):13191328 (1999); Garland et al., J. Immunol Meth. 227(1-2):53-63 (1999)).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in certain embodiments in the present disclosure.

In some embodiments, the T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment, the cells (for example, 10⁴ to 4×10⁸ T cells) and beads (for example, anti-CD3/CD28 MACSiBead particles at a recommended titer of 1:100) are combined in a buffer, preferably PBS (without divalent cations such as, calcium and magnesium). Those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and cell expansion. Further, use of high cell concentrations may allow more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment, the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α, or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### 7.3. Polypeptides Comprising a Functional Exogenous Receptor and Additional Domain(s)

In another aspect, provided herein is a polypeptide comprising at least one functional exogenous receptor (such as CAR, TCR and TAC) and at least one of p40 subunit of IL-12 and a ligand of CCR7 (such as CCL-19 and CCL-21).

In some embodiments, provided herein is a polypeptide comprising at least one functional exogenous receptor (such as CAR, TCR and TAC) and at least one of p40 subunit of IL-12 and CCL-19.

In some embodiments, provided herein is a polypeptide comprising a CAR and p40. In some embodiments, provided herein is a polypeptide comprising a CAR and CCL-19. In other embodiments, provided herein is a polypeptide comprising a CAR and both p40 and CCL-19. The CAR in the present polypeptide can be any of those described in Section 7.2 above. The p40 and CCL-19 in the present polypeptide can be any of those described in Section 7.2 above.

In some embodiments, provided herein is a polypeptide comprising a TCR and p40. In some embodiments, provided herein is a polypeptide comprising a TCR and CCL-19. In other embodiments, provided herein is a polypeptide comprising a TCR and both p40 and CCL-19. The p40 and CCL-19 in the present polypeptide can be any of those described in Section 7.2 above.

In some embodiments, provided herein is a polypeptide comprising a TAC and p40. In some embodiments, provided herein is a polypeptide comprising a TAC and CCL-19. In other embodiments, provided herein is a polypeptide comprising a TAC and both p40 and CCL-19. The p40 and CCL-19 in the present polypeptide can be any of those described in Section 7.2 above.

In some embodiments, p40 provided herein is a human p40 or a fragment or a variant thereof. In a specific embodiment, the p40 provided herein comprises the amino acid sequence of SEQ ID NO: 5. In some embodiments, the p40 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 5. In some embodiments, the p40 polypeptide provided herein is secreted. In some embodiments, the p40 polypeptide provided herein is membrane bound (e.g., MB12).

In some embodiments, CCL-19 provided herein is a human CCL-19 or a fragment or a variant thereof. In a specific embodiment, the CCL-19 provided herein comprises the amino acid sequence of SEQ ID NO: 6. In some embodiments, the CCL-19 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 6.

In the present polypeptides, the CAR, p40 and/or CCL-19 can be arranged in any order. For example, in the case wherein CAR, p40 and CCL-19 are all present in a polypeptide, in some embodiments, the polypeptide provided herein comprises from N terminus to C terminus: the CAR, p40 and CCL-19; in some embodiments, the polypeptide provided herein comprises from N terminus to C terminus: the CAR, CCL-19, and p40; in some embodiments, the polypeptide provided herein comprises from N terminus to C terminus: CCL-19, p40, and the CAR; in some embodiments, the polypeptide provided herein comprises from N terminus to C terminus: p40, CCL-19 and the CAR; in some embodiments, the polypeptide provided herein comprises from N terminus to C terminus: p40, the CAR, and CCL-19; and in some embodiments, the polypeptide provided herein comprises from N terminus to C terminus: CCL-19, the CAR, and p40.

In some embodiments of the various polypeptides provided herein, the CAR, p40 and/or CCL-19 are linked with each other via a peptide linker. In some embodiments, the peptide linker is a self-cleaving peptide such as 2A self-cleaving peptide, so that the CAR, p40 and/or CCL-19 become separate polypeptides upon cleavage in cells.

In other embodiments, provided herein is a polypeptide comprising at least one functional exogenous receptor (such as CAR, TCR and TAC) and at least one of p40 subunit of IL-12 and CCL-21.

In some embodiments, provided herein is a polypeptide comprising a CAR and p40. In some embodiments, provided herein is a polypeptide comprising a CAR and CCL-21. In other embodiments, provided herein is a polypeptide comprising a CAR and both p40 and CCL-21. The CAR in the present polypeptide can be any of those described in Section 7.2 above. The p40 and CCL-21 in the present polypeptide can be any of those described in Section 7.2 above.

In some embodiments, provided herein is a polypeptide comprising a TCR and p40. In some embodiments, provided herein is a polypeptide comprising a TCR and CCL-21. In other embodiments, provided herein is a polypeptide comprising a TCR and both p40 and CCL-21. The p40 and CCL-21 in the present polypeptide can be any of those described in Section 7.2 above.

In some embodiments, provided herein is a polypeptide comprising a TAC and p40. In some embodiments, provided herein is a polypeptide comprising a TAC and CCL-21. In other embodiments, provided herein is a polypeptide comprising a TAC and both p40 and CCL-21. The p40 and CCL-21 in the present polypeptide can be any of those described in Section 7.2 above.

In some embodiments, p40 provided herein is a human p40 or a fragment or a variant thereof. In a specific embodiment, the p40 provided herein comprises the amino acid sequence of SEQ ID NO: 5. In some embodiments, the p40 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 5. In some embodiments, the p40 polypeptide provided herein is secreted. In some embodiments, the p40 polypeptide provided herein is membrane bound (e.g., MB12).

In some embodiments, CCL-21 provided herein is a human CCL-21 or a fragment or a variant thereof. In a specific embodiment, the CCL-21 provided herein comprises the amino acid sequence of SEQ ID NO: 22. In some embodiments, the CCL-21 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 22.

In the present polypeptides, the CAR, p40 and/or CCL-21 can be arranged in any order. For example, in the case wherein CAR, p40 and CCL-21 are all present in a polypeptide, in some embodiments, the polypeptide provided herein comprises from N terminus to C terminus: the CAR, p40 and CCL-21; in some embodiments, the polypeptide provided herein comprises from N terminus to C terminus: the CAR, CCL-21, and p40; in some embodiments, the polypeptide provided herein comprises from N terminus to C terminus: CCL-21, p40, and the CAR; in some embodiments, the polypeptide provided herein comprises from N terminus to C terminus: p40, CCL-21 and the CAR; in some embodiments, the polypeptide provided herein comprises from N terminus to C terminus: p40, the CAR, and CCL-21; and in some embodiments, the polypeptide provided herein comprises from N terminus to C terminus: CCL-21, the CAR, and p40.

In some embodiments of the various polypeptides provided herein, the CAR, p40 and/or CCL-21 are linked with each other via a peptide linker. In some embodiments, the peptide linker is a self-cleaving peptide such as 2A self-cleaving peptide, so that the CAR, p40 and/or CCL-21 become separate polypeptides upon cleavage in cells.

The members of 2A peptides are named after the virus in which they have been first described. For example, F2A, the first described 2A peptide, is derived from foot-and-mouth disease virus. The self-cleaving 18-22 amino acids long 2A peptides mediate ‘ribosomal skipping’ between the proline and glycine residues and inhibit peptide bond formation without affecting downstream translation. These peptides allow multiple proteins to be encoded as polyproteins, which dissociate into component proteins upon translation. Self-cleaving peptides are found in members of the picomaviridae virus family, including aphthoviruses such as foot-and-mouth disease virus (FMDV), equine rhinitis A virus (ERAV), Thosea asigna virus (TaV) and porcine teschovirus-1 (PTV-1) (see Donnelly et al., J. Gen. Virol., 82: 1027-101 (2001); Ryan et al., J. Gen. Virol., 72: 2727-2732 (2001)) and cardioviruses such as theilovirus (e.g., theiler's murine encephalomyelitis) and encephalomyocarditis viruses. The 2A peptides derived from FMDV, ERAV, PTV-1, and TaV are sometimes referred to as “F2A,” “E2A,” “P2,” and “T2A,” respectively, and are included in the present disclosure, e.g., as described in Donnelly et al., J. Gen. Virol., 78: 13-21 (1997); Ryan and Drew, EMBO J., 13: 928-933 (1994); Szymczak et al., Nature Biotech., 5: 589-594 (2004); Hasegawa et al., Stem Cells, 25(7): 1707-12 (2007). In yet other embodiments, intein mediated protein splicing system is used herein, e.g., as described in Shah and Muir, Chem Sci., 5(1): 446461 (2014) and Topilina and Mills, Mobile DNA, 5 (5) (2014). Other methods known in the art can also be used in the present constructs.

In some embodiments, the 2A self-cleaving peptide is selected from a group consisting of F2A, E2A, P2A, T2A, or variants thereof. In some embodiments, the self-cleaving peptide is a 2A self-cleaving peptide P2A fragment comprising the amino acid sequence of SEQ ID NO: 13. In a specific embodiment, the self-cleaving peptide is T2A fragment comprising the amino acid sequence of SEQ ID NO: 14.

In some specific embodiments, p40 and CCL-19 are linked by a first self-cleaving peptide. In some embodiments, the first self-cleaving peptide is a 2A self-cleaving peptide T2A fragment comprising the amino acid sequence of SEQ ID NO: 14. In some embodiments, a domain comprising p40 subunit of IL-12 and CCL-19 provided herein comprises the amino acid sequence of SEQ ID NO: 4. In some embodiments, a domain comprising p40 subunit of IL-12 and CCL-19 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 4. In some embodiments, the functional exogenous receptor such as a CAR is linked to the domain by a second self-cleaving peptide. In some embodiments, the second self-cleaving peptide is a 2A self-cleaving peptide P2A fragment comprising the amino acid sequence of SEQ ID NO: 13.

In other specific embodiments, p40 and CCL-21 are linked by a first self-cleaving peptide. In some embodiments, the first self-cleaving peptide is a 2A self-cleaving peptide T2A fragment comprising the amino acid sequence of SEQ ID NO: 14. In some embodiments, a domain comprising p40 subunit of IL-12 and CCL-21 provided herein comprises the amino acid sequence of SEQ ID NO: 20. In some embodiments, a domain comprising p40 subunit of IL-12 and CCL-21 provided herein comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 20. In some embodiments, the functional exogenous receptor such as a CAR is linked to the domain by a second self-cleaving peptide. In some embodiments, the second self-cleaving peptide is a 2A self-cleaving peptide P2A fragment comprising the amino acid sequence of SEQ ID NO: 13.

In another aspect, provided herein is a nucleic acid encoding a polypeptide described above comprising a functional exogenous receptor (such as a CAR) provided herein, p40 subunit of IL-12, and/or a ligand of CCR7 (such as CCL-19 and CCL-21), which is described in more detail below.

### 7.4. Polynucleotides

In another aspect, the disclosure provides polynucleotides that encode the polypeptide provided herein, including those described in Section 7.2 and Section 7.3 above.

More specifically, in some embodiments, provided herein is a polynucleotide encoding a polypeptide comprising a chimeric antigen receptor (CAR), p40, and CCL-19, wherein the CAR, p40 and CCL-19 are fused with each other by peptide linkers, such as 2A self-cleaving peptide linkers.

In yet another aspect, provided herein is a polynucleotide comprising a region encoding a CAR, a region encoding p40, and/or a region encoding CCL-19. In some embodiments, two or more regions are controlled by the same promoter. For example, in some embodiments, internal ribosomal entry sites (IRES) are used herein to express multiple genes from one promoter. In other embodiments, these regions are controlled by separate promoters.

In yet another aspect, provided herein is a composition comprising a first polynucleotide encoding a CAR, a second polynucleotide encoding p40, and a third polynucleotide encoding CCL-19. In yet another aspect, provided herein is a composition comprising a first polynucleotide encoding a CAR, a second polynucleotide comprising a region encoding p40 and a region encoding CCL-19. In yet another aspect, provided herein is a composition comprising a first polynucleotide encoding p40, a second polynucleotide comprising a region encoding CAR and a region encoding CCL-19. In yet another aspect, provided herein is a composition comprising a first polynucleotide encoding CCL-19, a second polynucleotide comprising a region encoding CAR and a region encoding p40.

In yet another aspect, provided herein is a polynucleotide encoding a polypeptide comprising a TCR (or a subunit thereof), p40, and CCL-19, wherein the TCR (or a subunit thereof), p40 and CCL-19 are fused with each other by peptide linkers, such as 2A self-cleaving peptide linkers.

In yet another aspect, provided herein is a polynucleotide comprising a region encoding a TCR (or a subunit thereof), a region encoding p40, and/or a region encoding CCL-19. In some embodiments, two or more regions are controlled by the same promoter. For example, in some embodiments, internal ribosomal entry sites (IRES) are used herein to express multiple genes from one promoter. In other embodiments, these regions are controlled by separate promoters.

In yet another aspect, provided herein is a composition comprising a first polynucleotide encoding a TCR (or a subunit thereof), a second polynucleotide encoding p40, and a third polynucleotide encoding CCL-19. In yet another aspect, provided herein is a composition comprising a first polynucleotide encoding a TCR (or a subunit thereof), a second polynucleotide comprising a region encoding p40 and a region encoding CCL-19. In yet another aspect, provided herein is a composition comprising a first polynucleotide encoding p40, a second polynucleotide comprising a region encoding TCR (or a subunit thereof) and a region encoding CCL-19. In yet another aspect, provided herein is a composition comprising a first polynucleotide encoding CCL-19, a second polynucleotide comprising a region encoding TCR (or a subunit thereof) and a region encoding p40.

In yet another aspect, provided herein is a polynucleotide encoding a polypeptide comprising a TAC (or a domain thereof), p40, and CCL-19, wherein the TAC (or a domain thereof), p40 and CCL-19 are fused with each other by peptide linkers, such as 2A self-cleaving peptide linkers.

In yet another aspect, provided herein is a polynucleotide comprising a region encoding a TAC (or a domain thereof), a region encoding p40, and/or a region encoding CCL-19. In some embodiments, two or more regions are controlled by the same promoter. For example, in some embodiments, internal ribosomal entry sites (IRES) are used herein to express multiple genes from one promoter. In other embodiments, these regions are controlled by separate promoters.

In yet another aspect, provided herein is a composition comprising a first polynucleotide encoding a TAC (or a domain thereof), a second polynucleotide encoding p40, and a third polynucleotide encoding CCL-19. In yet another aspect, provided herein is a composition comprising a first polynucleotide encoding a TAC (or a domain thereof), a second polynucleotide comprising a region encoding p40 and a region encoding CCL-19. In yet another aspect, provided herein is a composition comprising a first polynucleotide encoding p40, a second polynucleotide comprising a region encoding TAC (or a domain thereof) and a region encoding CCL-19. In yet another aspect, provided herein is a composition comprising a first polynucleotide encoding CCL-19, a second polynucleotide comprising a region encoding TAC (or a domain thereof) and a region encoding p40.

In other embodiments, provided herein is a polynucleotide encoding a polypeptide comprising a chimeric antigen receptor (CAR), p40, and CCL-21, wherein the CAR, p40 and CCL-21 are fused with each other by peptide linkers, such as 2A self-cleaving peptide linkers.

In yet another aspect, provided herein is a polynucleotide comprising a region encoding a CAR, a region encoding p40, and/or a region encoding CCL-21. In some embodiments, two or more regions are controlled by the same promoter. For example, in some embodiments, internal ribosomal entry sites (IRES) are used herein to express multiple genes from one promoter. In other embodiments, these regions are controlled by separate promoters.

In yet another aspect, provided herein is a composition comprising a first polynucleotide encoding a CAR, a second polynucleotide encoding p40, and a third polynucleotide encoding CCL-21. In yet another aspect, provided herein is a composition comprising a first polynucleotide encoding a CAR, a second polynucleotide comprising a region encoding p40 and a region encoding CCL-21. In yet another aspect, provided herein is a composition comprising a first polynucleotide encoding p40, a second polynucleotide comprising a region encoding CAR and a region encoding CCL-21. In yet another aspect, provided herein is a composition comprising a first polynucleotide encoding CCL-21, a second polynucleotide comprising a region encoding CAR and a region encoding p40.

In yet another aspect, provided herein is a polynucleotide encoding a polypeptide comprising a TCR (or a subunit thereof), p40, and CCL-21, wherein the TCR (or a subunit thereof), p40 and CCL-21 are fused with each other by peptide linkers, such as 2A self-cleaving peptide linkers.

In yet another aspect, provided herein is a polynucleotide comprising a region encoding a TCR (or a subunit thereof), a region encoding p40, and/or a region encoding CCL-21. In some embodiments, two or more regions are controlled by the same promoter. For example, in some embodiments, internal ribosomal entry sites (IRES) are used herein to express multiple genes from one promoter. In other embodiments, these regions are controlled by separate promoters.

In yet another aspect, provided herein is a composition comprising a first polynucleotide encoding a TCR (or a subunit thereof), a second polynucleotide encoding p40, and a third polynucleotide encoding CCL-21. In yet another aspect, provided herein is a composition comprising a first polynucleotide encoding a TCR (or a subunit thereof), a second polynucleotide comprising a region encoding p40 and a region encoding CCL-21. In yet another aspect, provided herein is a composition comprising a first polynucleotide encoding p40, a second polynucleotide comprising a region encoding TCR (or a subunit thereof) and a region encoding CCL-21. In yet another aspect, provided herein is a composition comprising a first polynucleotide encoding CCL-21, a second polynucleotide comprising a region encoding TCR (or a subunit thereof) and a region encoding p40.

In yet another aspect, provided herein is a polynucleotide encoding a polypeptide comprising a TAC (or a domain thereof), p40, and CCL-21, wherein the TAC (or a domain thereof), p40 and CCL-21 are fused with each other by peptide linkers, such as 2A self-cleaving peptide linkers.

In yet another aspect, provided herein is a polynucleotide comprising a region encoding a TAC (or a domain thereof), a region encoding p40, and/or a region encoding CCL-21. In some embodiments, two or more regions are controlled by the same promoter. For example, in some embodiments, internal ribosomal entry sites (IRES) are used herein to express multiple genes from one promoter. In other embodiments, these regions are controlled by separate promoters.

In yet another aspect, provided herein is a composition comprising a first polynucleotide encoding a TAC (or a domain thereof), a second polynucleotide encoding p40, and a third polynucleotide encoding CCL-21. In yet another aspect, provided herein is a composition comprising a first polynucleotide encoding a TAC (or a domain thereof), a second polynucleotide comprising a region encoding p40 and a region encoding CCL-21. In yet another aspect, provided herein is a composition comprising a first polynucleotide encoding p40, a second polynucleotide comprising a region encoding TAC (or a domain thereof) and a region encoding CCL-21. In yet another aspect, provided herein is a composition comprising a first polynucleotide encoding CCL-21, a second polynucleotide comprising a region encoding TAC (or a domain thereof) and a region encoding p40.

The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the polypeptide of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the polypeptide of the disclosure. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

### 7.5. Vectors

Also provided are vectors comprising the polynucleotides or nucleic acid molecules described herein. In one embodiment, the nucleic acid molecules can be incorporated into a recombinant expression vector.

The present disclosure provides vectors for cloning and expressing any one of the polypeptides described herein. In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulator (such as immune checkpoint inhibitor) coding sequence and/or self-inactivating lentiviral vectors carrying chimeric antigen receptors can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector comprises any one of the nucleic acids encoding a polypeptide described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present disclosure. Promoters may be roughly categorized as constitutive promoters or regulated promoters, such as inducible promoters.

In some embodiments, the nucleic acid encoding the polypeptide is operably linked to a constitutive promoter. Constitutive promoters allow heterologous genes (also referred to as transgenes) to be expressed constitutively in the host cells. Exemplary constitutive promoters contemplated herein include, but are not limited to, murine stem cell virus (MSCV) promoter, cytomegalovirus (CMV) promoters, human elongation factors-1 alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1α, UbiC and PGK to drive chimeric antigen receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the nucleic acid encoding the CAR is operably linked to a hEF1α promoter. In some embodiments, the nucleic acid encoding the CAR is operably linked to a MSCV promoter.

In some embodiments, the nucleic acid encoding the polypeptide is operably linked to an inducible promoter. Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof.

In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

In some embodiments, the vector also contains a selectable marker gene or a reporter gene to select cells expressing the polypeptide from the population of host cells transfected through lentiviral vectors. Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

In some specific embodiments, the vector provided herein comprises a MND promoter (myeloproliferative sarcoma virus enhancer, negative control region deleted, dl587rev primer-binding site substituted (MND) promoter). In some specific embodiments, the vector provided herein comprises a MSCV promoter. In other specific embodiments, the vector provided herein comprises an EF1α

### 7.6. Methods of Making

In yet another aspect, provided herein is a method for making an engineered immune effector cell (e.g., a CAR-T cell or a TCR-T cell or a TAC-T cell), comprising introducing one or more polynucleotide(s) or the vector(s) provided herein (e.g., as described in Section 7.4 and Section 7.5 above) into an immune effector cell (e.g., a T cell).

More specifically, in some embodiments, CAR-T cells expressing exogenously introduced p40 and CCL-19 can be produced by introducing one or more nucleic acid(s) encoding a polypeptide described in Section 7.3 or one or more nucleic acid(s) described in Section 7.4 into T cells.

The CAR, p40 and CCL-19 can each be introduced into T cells separately as separately polypeptides. For example, a nucleic acid encoding a CAR provided herein, a nucleic acid encoding p40, and a nucleic acid encoding CCL-19 are introduced into T cells separately. Alternatively, any two of the three or all three of them can be introduced into T cell together as single polypeptide via one nucleic acid which will be cleaved upon translation in cells. For example, a nucleic acid encoding a polypeptide comprising a CAR provided herein and p40 linked via a self-cleaving peptide linker is introduced into the T cells, and separately a nucleic acid encoding CCL-19 is introduced into the T cells. Similarly, a nucleic acid encoding a polypeptide comprising a CAR provided herein and CCL-19 linked via a self-cleaving peptide linker is introduced into the T cells, and separately a nucleic acid encoding p40 is introduced into the T cells. In some embodiments, a nucleic acid encoding a polypeptide comprising all three of the CAR, p40 and CCL-19 linked to each other via self-cleaving peptide linkers can be introduced into T cells. Self-cleaving peptide linkers are described in more detail above. In some embodiments, the 2A self-cleaving peptide is selected from a group consisting of F2A, E2A, P2A, T2A, or variants thereof. In some embodiments, the self-cleaving peptide is a 2A self-cleaving peptide P2A fragment comprising the amino acid sequence of SEQ ID NO: 13. In a specific embodiment, the self-cleaving peptide is T2A fragment comprising the amino acid sequence of SEQ ID NO: 14.

Alternatively, the CAR-T cells provided herein can be produced by a polynucleotide comprising multiple regions, for example, a region encoding a CAR, a region encoding p40, and/or a region encoding CCL-19. Different regions can be controlled by the same promoter. For example, in some embodiments, internal ribosomal entry sites (IRES) are used herein to express multiple genes from one promoter. In other embodiments, different regions are controlled by separate promoters.

In yet another aspect, provided herein is an engineered immune effector cell (e.g., a CAR-T cell) produced according to the method provided herein. Among other properties, the CAR-T cells expressing exogenously introduced p40 and CCL-19 have higher level of IL-23 as compared to CAR-T cells without exogenously introduced p40 and CCL-19. In some embodiments, the level of IL-23 is at least 20% higher. In some embodiments, the level of IL-23 is at least 30% higher. In some embodiments, the level of IL-23 is at least 40% higher. In some embodiments, the level of IL-23 is at least 50% higher. In some embodiments, the level of IL-23 is at least 60% higher. In some embodiments, the level of IL-23 is at least 70% higher. In some embodiments, the level of IL-23 is at least 80% higher. In some embodiments, the level of IL-23 is at least 90% higher. In some embodiments, the level of IL-23 is at least 2 fold, 3 fold, 4 fold, 5 fold or higher.

In other embodiments, CAR-T cells expressing exogenously introduced p40 and CCL-21 can be produced by introducing one or more nucleic acid(s) encoding a polypeptide described in Section 7.3 or one or more nucleic acid(s) described in Section 7.4 into T cells.

The CAR, p40 and CCL-21 can each be introduced into T cells separately as separately polypeptides. For example, a nucleic acid encoding a CAR provided herein, a nucleic acid encoding p40, and a nucleic acid encoding CCL-21 are introduced into T cells separately. Alternatively, any two of the three or all three of them can be introduced into T cell together as single polypeptide via one nucleic acid which will be cleaved upon translation in cells. For example, a nucleic acid encoding a polypeptide comprising a CAR provided herein and p40 linked via a self-cleaving peptide linker is introduced into the T cells, and separately a nucleic acid encoding CCL-21 is introduced into the T cells. Similarly, a nucleic acid encoding a polypeptide comprising a CAR provided herein and CCL-21 linked via a self-cleaving peptide linker is introduced into the T cells, and separately a nucleic acid encoding p40 is introduced into the T cells. In some embodiments, a nucleic acid encoding a polypeptide comprising all three of the CAR, p40 and CCL-21 linked to each other via self-cleaving peptide linkers can be introduced into T cells. Self-cleaving peptide linkers are described in more detail above. In some embodiments, the 2A self-cleaving peptide is selected from a group consisting of F2A, E2A, P2A, T2A, or variants thereof. In some embodiments, the self-cleaving peptide is a 2A self-cleaving peptide P2A fragment comprising the amino acid sequence of SEQ ID NO: 13. In a specific embodiment, the self-cleaving peptide is T2A fragment comprising the amino acid sequence of SEQ ID NO: 14.

Alternatively, the CAR-T cells provided herein can be produced by a polynucleotide comprising multiple regions, for example, a region encoding a CAR, a region encoding p40, and/or a region encoding CCL-21. Different regions can be controlled by the same promoter. For example, in some embodiments, internal ribosomal entry sites (IRES) are used herein to express multiple genes from one promoter. In other embodiments, different regions are controlled by separate promoters.

In yet another aspect, provided herein is an engineered immune effector cell (e.g., a CAR-T cell) produced according to the method provided herein. Among other properties, the CAR-T cells expressing exogenously introduced p40 and CCL-21 have higher level of IL-23 as compared to CAR-T cells without exogenously introduced p40 and CCL-21. In some embodiments, the level of IL-23 is at least 20% higher. In some embodiments, the level of IL-23 is at least 30% higher. In some embodiments, the level of IL-23 is at least 40% higher. In some embodiments, the level of IL-23 is at least 50% higher. In some embodiments, the level of IL-23 is at least 60% higher. In some embodiments, the level of IL-23 is at least 70% higher. In some embodiments, the level of IL-23 is at least 80% higher. In some embodiments, the level of IL-23 is at least 90% higher. In some embodiments, the level of IL-23 is at least 2 fold, 3 fold, 4 fold, 5 fold or higher.

The methods described above in the context of CAR-T cells are also applicable to production of other immune effector cells such as TCR-T cells and TAC-T cells.

### 7.7. Pharmaceutical Compositions

In one aspect, the present disclosure further provides pharmaceutical compositions comprising an engineered cell of the present disclosure. In some embodiments, a pharmaceutical composition comprises a therapeutically effective amount of the engineered T cell of the present disclosure and a pharmaceutically acceptable excipient.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, PA. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers may be used to control the pH in a range which optimizes the therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives may be added to retard microbial growth. Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein or antibody. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The pharmaceutical compositions described herein may also contain more than one active compound or agent as necessary for the particular indication being treated. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

Various compositions and delivery systems are known and can be used with the therapeutic agents provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

In some embodiments, the pharmaceutical composition provided herein contains the binding molecules and/or cells in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

### 7.8. Methods and Uses

In another aspect, provided herein are methods for using and uses of the engineered cells provided herein (e.g., CAR-T cells). Such methods and uses include therapeutic methods and uses, for example, involving administration of the cells, or compositions containing the same, to a subject having a disease or disorder. In some embodiments, the cell is administered in an effective amount to effect treatment of the disease or disorder. Uses include uses of the cells in such methods and treatments, and in the preparation of a medicament in order to carry out such therapeutic methods. In some embodiments, the methods are carried out by administering the cells, or compositions comprising the same, to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed. In other embodiments, the method or the use provided herein prevents a disease or disorder.

In some embodiments, the present CAR-T cell therapies are used for treating solid tumor cancer. In other embodiments, the present CAR-T cell therapies are used for treating blood cancer. In other embodiments, the disease or disorder is an autoimmune and inflammatory disease. In other embodiments, the disease or disorder is an infectious disease.

In some embodiments, the disease or disorder is a disease of abnormal cell growth and/or dysregulated apoptosis. Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments, the disease or disorder is selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer and spleen cancer.

In some embodiments, the disease or disorder is a hematological cancer, such as leukemia, lymphoma, or myeloma. In some embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the disease or disorder is a solid tumor cancer. In some embodiments, the solid tumor cancer is selected from a group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, a liver cancer and a lung cancer.

In some embodiments, the cancer is an adrenal cancer. In some embodiments, the cancer is an anal cancer. In some embodiments, the cancer is an appendix cancer. In some embodiments, the cancer is a bile duct cancer. In some embodiments, the cancer is a bladder cancer. In some embodiments, the cancer is a bone cancer. In some embodiments, the cancer is a brain cancer. In some embodiments, the cancer is a breast cancer. In some embodiments, the cancer is a cervical cancer. In some embodiments, the cancer is a colorectal cancer. In some embodiments, the cancer is an esophageal cancer. In some embodiments, the cancer is a gallbladder cancer. In some embodiments, the cancer is a gestational trophoblastic. In some embodiments, the cancer is a head and neck cancer. In some embodiments, the cancer is a Hodgkin lymphoma. In some embodiments, the cancer is an intestinal cancer. In some embodiments, the cancer is a kidney cancer. In some embodiments, the cancer is a leukemia. In some embodiments, the cancer is a liver cancer. In some embodiments, the cancer is a lung cancer. In some embodiments, the cancer is a melanoma. In some embodiments, the cancer is a mesothelioma. In some embodiments, the cancer is a multiple myeloma (MM). In some embodiments, the cancer is a neuroendocrine tumor. In some embodiments, the cancer is a non-Hodgkin lymphoma. In some embodiments, the cancer is an oral cancer. In some embodiments, the cancer is an ovarian cancer. In some embodiments, the cancer is a pancreatic cancer. In some embodiments, the cancer is a prostate cancer. In some embodiments, the cancer is a sinus cancer. In some embodiments, the cancer is a skin cancer. In some embodiments, the cancer is a soft tissue sarcoma spinal cancer. In some embodiments, the cancer is a stomach cancer. In some embodiments, the cancer is a testicular cancer. In some embodiments, the cancer is a throat cancer. In some embodiments, the cancer is a thyroid cancer. In some embodiments, the cancer is a uterine cancer endometrial cancer. In some embodiments, the cancer is a vaginal cancer. In some embodiments, the cancer is a vulvar cancer.

In some embodiments, the adrenal cancer is an adrenocortical carcinoma (ACC), adrenal cortex cancer, pheochromocytoma, or neuroblastoma. In some embodiments, the anal cancer is a squamous cell carcinoma, cloacogenic carcinoma, adenocarcinoma, basal cell carcinoma, or melanoma. In some embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments, the bladder cancer is transitional cell carcinoma (TCC), papillary carcinoma, flat carcinoma, squamous cell carcinoma, adenocarcinoma, small-cell carcinoma, or sarcoma. In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments, the cervical cancer is a squamous cell carcinoma, or adenocarcinoma. In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments, the esophageal cancer is an adenocarcinoma or squamous cell carcinoma. In some embodiments, the gall bladder cancer is an adenocarcinoma, papillary adenocarcinoma, adenosquamous carcinoma, squamous cell carcinoma, small cell carcinoma, or sarcoma. In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments, the intestinal cancer is a small intestine cancer, small bowel cancer, adenocarcinoma, sarcoma, gastrointestinal stromal tumors, carcinoid tumors, or lymphoma. In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML. In some embodiments, the liver cancer is a hepatocellular carcinoma (HCC), fibrolamellar HCC, cholangiocarcinoma, angiosarcoma, or liver metastasis. In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments, the mesothelioma is a pleural mesothelioma, peritoneal mesothelioma, pericardial mesothelioma, or testicular mesothelioma. In some embodiments, the multiple myeloma is an active myeloma or smoldering myeloma. In some embodiments, the neuroendocrine tumor is a gastrointestinal neuroendocrine tumor, pancreatic neuroendocrine tumor, or lung neuroendocrine tumor. In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments, the pancreatic cancer is a pancreatic exocrine gland cancer, pancreatic endocrine gland cancer, or pancreatic adenocarcinoma, islet cell tumor, or neuroendocrine tumor. In some embodiments, the prostate cancer is a prostate adenocarcinoma, prostate sarcoma, transitional cell carcinoma, small cell carcinoma, or neuroendocrine tumor. In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments, the spinal cancer is a spinal metastatic tumor. In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments, the thyroid cancer is a papillary carcinoma, follicular carcinoma, Hürthle cell carcinoma, medullary thyroid carcinoma, or anaplastic carcinoma. In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments, the vaginal cancer is a squamous cell carcinoma, adenocarcinoma, melanoma, or sarcoma. In some embodiments, the vulvar cancer is a squamous cell carcinoma or adenocarcinoma.

In some embodiments, the disease or disorder is caused by a pathogen. In some embodiments, the pathogen causes an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection. In some embodiments, the infectious disease is Acute Flaccid Myelitis (AFM). In some embodiments, the infectious disease is Anaplasmosis. In some embodiments, the infectious disease is Anthrax. In some embodiments, the infectious disease is Babesiosis. In some embodiments, the infectious disease is Botulism. In some embodiments, the infectious disease is Brucellosis. In some embodiments, the infectious disease is Campylobacteriosis. In some embodiments, the infectious disease is Carbapenem-resistant Infection. In some embodiments, the infectious disease is Chancroid. In some embodiments, the infectious disease is Chikungunya Virus Infection. In some embodiments, the infectious disease is Chlamydia. In some embodiments, the infectious disease is Ciguatera. In some embodiments, the infectious disease is Difficile Infection. In some embodiments, the infectious disease is Perfringens. In some embodiments, the infectious disease is Coccidioidomycosis fungal infection. In some embodiments, the infectious disease is coronavirus. In some embodiments, the infectious disease is Covid-19 (SARS-CoV-2). In some embodiments, the infectious disease is Creutzfeldt-Jacob Disease/transmissible spongiform encephalopathy. In some embodiments, the infectious disease is Cryptosporidiosis (Crypto). In some embodiments, the infectious disease is Cyclosporiasis. In some embodiments, the infectious disease is Dengue 1, 2, 3 or 4. In some embodiments, the infectious disease is Diphtheria. In some embodiments, the infectious disease is E. coli infection/Shiga toxin-producing (STEC). In some embodiments, the infectious disease is Eastern Equine Encephalitis. In some embodiments, the infectious disease is Hemorrhagic Fever (Ebola). In some embodiments, the infectious disease is Ehrlichiosis. In some embodiments, the infectious disease is Encephalitis. In some embodiments, the infectious disease is Arboviral or parainfectious. In some embodiments, the infectious disease is Non-Polio Enterovirus. In some embodiments, the infectious disease is D68 Enteroviru (EV-D68). In some embodiments, the infectious disease is Giardiasis. In some embodiments, the infectious disease is Glanders. In some embodiments, the infectious disease is Gonococcal Infection. In some embodiments, the infectious disease is Granuloma inguinale. In some embodiments, the infectious disease is Haemophilus influenza disease Type B (Hib or H-flu). In some embodiments, the infectious disease is Hantavirus Pulmonary Syndrome (HPS). In some embodiments, the infectious disease is Hemolytic Uremic Syndrome (HUS). In some embodiments, the infectious disease is Hepatitis A (Hep A). In some embodiments, the infectious disease is Hepatitis B (Hep B). In some embodiments, the infectious disease is Hepatitis C (Hep C). In some embodiments, the infectious disease is Hepatitis D (Hep D). In some embodiments, the infectious disease is Hepatitis E (Hep E). In some embodiments, the infectious disease is Herpes. In some embodiments, the infectious disease is Herpes Zoster (Shingles). In some embodiments, the infectious disease is Histoplasmosis infection. In some embodiments, the infectious disease is Human Immunodeficiency Virus/AIDS (HIV/AIDS). In some embodiments, the infectious disease is Human Papillomavirus (HPV). In some embodiments, the infectious disease is influenza (Flu). In some embodiments, the infectious disease is Legionellosis (Legionnaires Disease). In some embodiments, the infectious disease is Leprosy (Hansens Disease). In some embodiments, the infectious disease is Leptospirosis. In some embodiments, the infectious disease is Listeriosis (Listeria). In some embodiments, the infectious disease is Lyme Disease. In some embodiments, the infectious disease is Lymphogranuloma venereum infection (LGV). In some embodiments, the infectious disease is Malaria. In some embodiments, the infectious disease is Measles. In some embodiments, the infectious disease is Melioidosis. In some embodiments, the infectious disease is Meningitis (Viral). In some embodiments, the infectious disease is Meningococcal Disease (Meningitis (Bacterial)). In some embodiments, the infectious disease is Middle East Respiratory Syndrome Coronavirus (MERS-CoV). In some embodiments, the infectious disease is Mumps. In some embodiments, the infectious disease is Norovirus. In some embodiments, the infectious disease is Pediculosis. In some embodiments, the infectious disease is Pelvic Inflammatory Disease (PID). In some embodiments, the infectious disease is Pertussis (Whooping Cough). In some embodiments, the infectious disease is Plague (Bubonic. In some embodiments, the infectious disease is Septicemic. In some embodiments, the infectious disease is Pneumonic). In some embodiments, the infectious disease is Pneumococcal Disease (Pneumonia). In some embodiments, the infectious disease is Poliomyelitis (Polio). In some embodiments, the infectious disease is Powassan. In some embodiments, the infectious disease is Psittacosis. In some embodiments, the infectious disease is Pthiriasis. In some embodiments, the infectious disease is Pustular Rash diseases (Small pox. In some embodiments, the infectious disease is monkeypox. In some embodiments, the infectious disease is cowpox). In some embodiments, the infectious disease is Q-Fever. In some embodiments, the infectious disease is Rabies. In some embodiments, the infectious disease is Rickettsiosis (Rocky Mountain Spotted Fever). In some embodiments, the infectious disease is Rubella (German Measles). In some embodiments, the infectious disease is Salmonellosis gastroenteritis (Salmonella). In some embodiments, the infectious disease is Scabies. In some embodiments, the infectious disease is Scombroid. In some embodiments, the infectious disease is Sepsis. In some embodiments, the infectious disease is Severe Acute Respiratory Syndrome (SARS). In some embodiments, the infectious disease is Shigellosis gastroenteritis (Shigella). In some embodiments, the infectious disease is Smallpox. In some embodiments, the infectious disease is Staphyloccal Infection Methicillin-resistant (MRSA). In some embodiments, the infectious disease is Staphylococcal Food Poisoning Enterotoxin B Poisoning (Staph Food Poisoning). In some embodiments, the infectious disease is Saphylococcal Infection Vancomycin Intermediate (VISA). In some embodiments, the infectious disease is Staphylococcal Infection Vancomycin Resistant (VRSA). In some embodiments, the infectious disease is Streptococcal Disease Group A (invasive) (Strep A (invasive). In some embodiments, the infectious disease is Streptococcal Disease. In some embodiments, the infectious disease is Group B (Strep-B). In some embodiments, the infectious disease is Streptococcal Toxic-Shock Syndrome STSS Toxic Shock. In some embodiments, the infectious disease is Syphilis (primary. In some embodiments, the infectious disease is secondary. In some embodiments, the infectious disease is early latent. In some embodiments, the infectious disease is late latent. In some embodiments, the infectious disease is congenital). In some embodiments, the infectious disease is Tetanus Infection. In some embodiments, the infectious disease is Trichomoniasis. In some embodiments, the infectious disease is Trichonosis Infection. In some embodiments, the infectious disease is Tuberculosis (TB). In some embodiments, the infectious disease is Tuberculosis Latent (LTBI). In some embodiments, the infectious disease is Tularemia. In some embodiments, the infectious disease is Typhoid Fever Group D. In some embodiments, the infectious disease is Vaginosis. In some embodiments, the infectious disease is Varicella (Chickenpox), Vibrio cholerae (Cholera). In some embodiments, the infectious disease is Vibriosis (Vibrio). In some embodiments, the infectious disease is Ebola Virus Hemorrhagic Fever. In some embodiments, the infectious disease is Lasa Virus Hemorrhagic Fever. In some embodiments, the infectious disease is Marburg Virus Hemorrhagic Fever. In some embodiments, the infectious disease is West Nile Virus. In some embodiments, the infectious disease is Yellow Fever. In some embodiments, the infectious disease is Yersenia. In some embodiments, the infectious disease is and Zika Virus Infection.

In some embodiments, the pathogen is a bacteria. In some embodiments, the bacteria is a bacteria of a Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio or Yersinia genus. In some embodiments, the bacteria is a bacteria of the Bacillus genus. In some embodiments, the bacteria is a bacteria of the Bartonella genus. In some embodiments, the bacteria is a bacteria of the Bordetella genus. In some embodiments, the bacteria is a bacteria of the Borrelia genus. In some embodiments, the bacteria is a bacteria of the Brucella genus. In some embodiments, the bacteria is a bacteria of the Campylobacter genus. In some embodiments, the bacteria is a bacteria of the Chlamydia genus. In some embodiments, the bacteria is a bacteria of the Chlamydophila genus. In some embodiments, the bacteria is a bacteria of the clostridium genus. In some embodiments, the bacteria is a bacteria of the Corynebacterium genus. In some embodiments, the bacteria is a bacteria of the Enterococcus genus. In some embodiments, the bacteria is a bacteria of the Escherichia genus. In some embodiments, the bacteria is a bacteria of the Francisella genus. In some embodiments, the bacteria is a bacteria of the Haemophilus genus. In some embodiments, the bacteria is a bacteria of the Helicobacter genus. In some embodiments, the bacteria is a bacteria of the Legionella genus. In some embodiments, the bacteria is a bacteria of the leptospira genus. In some embodiments, the bacteria is a bacteria of the Listeria genus. In some embodiments, the bacteria is a bacteria of the Mycobacterium genus. In some embodiments, the bacteria is a bacteria of the Mycoplasma genus. In some embodiments, the bacteria is a bacteria of the Neisseria genus. In some embodiments, the bacteria is a bacteria of the Pseudomonas genus. In some embodiments, the bacteria is a bacteria of the Rickettsia genus. In some embodiments, the bacteria is a bacteria of the Salmonella genus. In some embodiments, the bacteria is a bacteria of the Shigella genus. In some embodiments, the bacteria is a bacteria of the Staphylococcus genus. In some embodiments, the bacteria is a bacteria of the Streptococcus genus. In some embodiments, the bacteria is a bacteria of the Treponema genus. In some embodiments, the bacteria is a bacteria of the Ureaplasma genus. In some embodiments, the bacteria is a bacteria of the Vibrio genus. In some embodiments, the bacteria is a bacteria of the Yersinia genus.

In some embodiments, the pathogen is a parasite. In some embodiments, the parasite is a protozoa, helminth, or ectoparasite. In some embodiments, the protozoa is an Entamoeba, Giardia, Leishmania, Balantidium, Plasmodium, or Cryptosporidium. In some embodiments, the helminth is a trematode, cestode, acanthocephalan, or round worm. In some embodiments, the ectoparasite is an arthropod.

In some embodiments, the pathogen is a virus. In some embodiments, the virus is a virus of the adenoviridae, arenaviridae, astroviridae, bunyaviridae, caliciviridae, coronaviridae, filoviridae, flaviviridae, hepadnaviridae, hepeviridae, orthomyxoviridae, papillomaviridae, paramyxoviridae, parvoviridae, picomaviridae, polyomaviridae, poxviridae, reoviridae, retroviridae, rhabdoviridae, or togaviridae family. In some embodiments family. In some embodiments, the virus is a virus of the virus is a virus of the adenoviridae family. In some embodiments, the virus is a virus of the arenaviridae family. In some embodiments, the virus is a virus of the astroviridae family. In some embodiments, the virus is a virus of the bunyaviridae family. In some embodiments, the virus is a virus of the caliciviridae family. In some embodiments, the virus is a virus of the coronaviridae family. In some embodiments, the virus is a virus of the filoviridae family. In some embodiments, the virus is a virus of the flaviviridae family. In some embodiments, the virus is a virus of the hepadnaviridae family. In some embodiments, the virus is a virus of the hepeviridae family. In some embodiments, the virus is a virus of the orthomyxoviridae family. In some embodiments, the virus is a virus of the papillomaviridae family. In some embodiments, the virus is a virus of the paramyxoviridae family. In some embodiments, the virus is a virus of the parvoviridae family. In some embodiments, the virus is a virus of the picomaviridae family. In some embodiments, the virus is a virus of the polyomaviridae family. In some embodiments, the virus is a virus of the poxviridae family. In some embodiments, the virus is a virus of the reoviridae family. In some embodiments, the virus is a virus of the retroviridae family. In some embodiments, the virus is a virus of the rhabdoviridae family. In some embodiments, the virus is a virus of the togaviridae family.

In some embodiments, the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus. In some embodiments, the virus is an adenovirus. In some embodiments, the virus is a coronavirus. In some embodiments, the coronavirus virus is Covid-19 (SARS-CoV-2). In some embodiments, the virus is a coxsackievirus. In some embodiments, the virus is a Epstein-Barr virus. In some embodiments, the virus is a hepatitis A virus. In some embodiments, the virus is a hepatitis B virus. In some embodiments, the virus is a hepatitis C virus. In some embodiments, the virus is a herpes simplex virus type 2. In some embodiments, the virus is a cytomegalovirus. In some embodiments, the virus is a human herpes virus type 8. In some embodiments, the virus is a human immunodeficiency virus. In some embodiments, the virus is an influenza virus. In some embodiments, the virus is a measles virus. In some embodiments, the virus is a mumps virus. In some embodiments, the virus is a human papillomavirus. In some embodiments, the virus is a parainfluenza virus. In some embodiments, the virus is a poliovirus. In some embodiments, the virus is a rabies virus. In some embodiments, the virus is a respiratory syncytial virus. In some embodiments, the virus is a rubella virus. In some embodiments, the virus is a varicella-zoster virus.

In other embodiments, the disease or disorder is an immune or autoimmune disorder. Such disorders include autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome, AIDS dementia complex, alcoholic cirrhosis, alcohol-induced liver injury, alcohol-induced hepatitis, allergic conjunctivitis, allergic contact dermatitis, allergic rhinitis, allergy and asthma, allograft rejection, alpha-1-antitrypsin deficiency, Alzheimer's disease, amyotrophic lateral sclerosis, anemia, angina pectoris, ankylosing spondylitis-associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemo lytic anaemia, autoimmune hepatitis, type-1 autoimmune hepatitis (classical autoimmune or lupoid hepatitis), autoimmune mediated hypoglycemia, autoimmune neutropenia, autoimmune thrombocytopenia, autoimmune thyroid disease, B-cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy-associated disorders, Chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinemia), conjunctivitis, connective tissue disease-associated interstitial lung disease, contact dermatitis, Coombs-positive hemolytic anemia, cor pulmonale, Creutzfeldt-Jakob disease, cryptogenic autoimmune hepatitis, cryptogenic fibrosing alveolitis, culture-negative sepsis, cystic fibrosis, cytokine therapy-associated disorders, Crohn's disease, dementia pugilistica, demyelinating diseases, dengue hemorrhagic fever, dermatitis, dermatitis scleroderma, dermatologic conditions, dermatomyositis/polymyositis-associated lung disease, diabetes, diabetic arteriosclerotic disease, diabetes mellitus, diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hemophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram-negative sepsis, gram-positive sepsis, granulomas due to intracellular organisms, group B streptococci (GBS) infection, Graves' disease, hemosiderosis-associated lung disease, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopenic purpura, hemorrhage, Henoch-Schoenlein purpura, hepatitis A, hepatitis B, hepatitis C, HIV infection/HIV neuropathy, Hodgkin's disease, hypoparathyroidism, Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopenia, idiopathic pulmonary fibrosis, idiopathic thrombocytopenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anemia, juvenile rheumatoid arthritis, juvenile spinal muscular atrophy, Kaposi's sarcoma, Kawasaki's disease, kidney transplant rejection, Legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease-associated lung disease, monoclonal gammopathy, multiple myeloma, multiple systems degenerations (Mencel, Dejerine-Thomas, Shy-Drager and Machado-Joseph), myalgic encephalitis/Royal Free Disease, myasthenia gravis, microscopic vasculitis of the kidneys, Mycobacterium avium intracellulare, Mycobacterium tuberculosis, myelodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, non-alcoholic steatohepatitis, occlusion of the abdominal aorta and its branches, occlusive arterial disorders, organ transplant rejection, orchitis/epididymitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, Pemphigus vulgaris, Pemphigus foliaceus, pemphigoid, perennial rhinitis, pericardial disease, peripheral atherosclerotic disease, peripheral vascular disorders, peritonitis, pernicious anemia, phacogenic uveitis, Pneumocystis carinii pneumonia, pneumonia, POEMS syndrome (polyneuropathy, organomegaly, endocrinopathy, monoclonal gammopathy, and skin changes syndrome), post-perfusion syndrome, post-pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, progressive supranuclear palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynoud's disease, Refsum's disease, regular narrow QRS tachycardia, Reiter's disease, renal disease NOS, renovascular hypertension, reperfusion injury, restrictive cardiomyopathy, rheumatoid arthritis-associated interstitial lung disease, rheumatoid spondylitis, sarcoidosis, Schmidt's syndrome, scleroderma, senile chorea, senile dementia of Lewy body type, sepsis syndrome, septic shock, seronegative arthropathies, shock, sickle cell anemia, T-cell or FAB ALL, Takayasu's disease/arteritis, telangiectasia, Th2-type and Th1-type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemophagocytic syndrome, Wegener's granulomatosis, Wernicke-Korsakoff syndrome, Wilson's disease, xenograft rejection of any organ or tissue, Yersinia and Salmonella-associated arthropathy, acquired immunodeficiency disease syndrome (AIDS), autoimmune lymphoproliferative syndrome, hemolytic anemia, inflammatory diseases, thrombocytopenia, acute and chronic immune diseases associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis, Lyme arthritis, psoriatic arthritis and reactive arthritis), Sjogren's disease-associated lung disease, Sjogren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, sporadic polyglandular deficiency type I, sporadic polyglandular deficiency type II, Still's disease, streptococcal myositis, stroke, structural lesions of the cerebellum, subacute sclerosing panencephalitis, sympathetic ophthalmia, syncope, syphilis of the cardiovascular system, systemic anaphylaxis, systemic inflammatory response syndrome, systemic onset juvenile rheumatoid arthritis, systemic lupus erythematosus, systemic lupus erythematosus-associated lung disease, lupus nephritis, systemic sclerosis, and systemic sclerosis-associated interstitial lung disease.

In some embodiments, the disease or disorder is an inflammatory disease. Inflammation plays a fundamental role in host defenses and the progression of immune-mediated diseases. The inflammatory response is initiated in response to injury (e.g., trauma, ischemia, and foreign particles) and infection (e.g., bacterial or viral infection) by a complex cascade of events, including chemical mediators (e.g., cytokines and prostaglandins) and inflammatory cells (e.g., leukocytes). The inflammatory response is characterized by increased blood flow, increased capillary permeability, and the influx of phagocytic cells. These events result in swelling, redness, warmth (altered heat patterns), and pus formation at the site of injury or infection.

Cytokines and prostaglandins control the inflammatory response, and are released in an ordered and self-limiting cascade into the blood or affected tissues. This release of cytokines and prostaglandins increases the blood flow to the area of injury or infection, and may result in redness and warmth. Some of these chemicals cause a leak of fluid into the tissues, resulting in swelling. This protective process may stimulate nerves and cause pain. These changes, when occurring for a limited period in the relevant area, work to the benefit of the body.

A delicate well-balanced interplay between the humoral and cellular immune elements in the inflammatory response enables the elimination of harmful agents and the initiation of the repair of damaged tissue. When this delicately balanced interplay is disrupted, the inflammatory response may result in considerable damage to normal tissue and may be more harmful than the original insult that initiated the reaction. In these cases of uncontrolled inflammatory responses, clinical intervention is needed to prevent tissue damage and organ dysfunction. Diseases such as psoriasis, rheumatoid arthritis, osteoarthritis, psoriatic arthritis, Crohn's disease, asthma, allergies or inflammatory bowel disease, are characterized by chronic inflammation. Inflammatory diseases such as arthritis, related arthritic conditions (e.g., osteoarthritis, rheumatoid arthritis, and psoriatic arthritis), inflammatory bowel disease (e.g., Crohn's disease and ulcerative colitis), sepsis, psoriasis, atopic dermatitis, contact dermatitis, and chronic obstructive pulmonary disease, chronic inflammatory pulmonary diseases are also prevalent and problematic ailments.

In some embodiments, the methods include adoptive cell therapy, whereby genetically engineered cells are administered to a subject. Such administration can promote activation of the cells (e.g., T cell activation), such that the cells of the disease or disorder are targeted for destruction.

In some embodiments, the methods include administration of the cells or a composition containing the cells to a subject, tissue, or cell, such as one having, at risk for, or suspected of having the disease or disorder. In some embodiments, the cells, populations, and compositions are administered to a subject having the particular disease or disorder to be treated, e.g., via adoptive cell therapy, such as adoptive T cell therapy. In some embodiments, the cells or compositions are administered to the subject, such as a subject having or at risk for the disease or disorder. In some embodiments, the methods thereby treat, e.g., ameliorate one or more symptom of the disease or disorder.

Methods for administration of cells for adoptive cell therapy are known, as described, e.g., in US Patent Application Publication No. 2003/0170238; U.S. Pat. No. 4,690,915; Rosenberg, Nat Rev Clin Oncol. 8 (10):577-85 (2011); Themeli et al., Nat Biotechnol. 31(10): 928-933 (2013); Tsukahara et al., Biochem Biophys Res Commun 438(1): 84-9 (2013); and Davila et al., PLoS ONE 8(4): e61338 (2013). These methods may be used in connection with the methods and compositions provided herein.

In some embodiments, the cell therapy (e.g., adoptive T cell therapy) is carried out by autologous transfer, in which the cells are isolated and/or otherwise prepared from the subject who is to receive the cell therapy, or from a sample derived from such a subject. Thus, in some aspects, the cells are derived from a subject in need of a treatment and the cells, following isolation and processing are administered to the same subject. In other embodiments, the cell therapy (e.g., adoptive T cell therapy) is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. The subject can be male or female and can be any suitable age, including infant, juvenile, adolescent, adult, and geriatric subjects. In some examples, the subject is a validated animal model for disease, adoptive cell therapy, and/or for assessing toxic outcomes.

The composition provided herein can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration.

The amount of a prophylactic or therapeutic agent provided herein that will be effective in the prevention and/or treatment of a disease or condition can be determined by standard clinical techniques. Effective doses may be extrapolated from dose-response curves derived from in vitro or animal model test systems. For the prevention or treatment of disease, the appropriate dosage of the binding molecule or cell may depend on the type of disease or disorder to be treated, the type of binding molecule, the severity and course of the disease or disorder, whether the therapeutic agent is administered for preventive or therapeutic purposes, previous therapy, the patient's clinical history and response to the agent, and the discretion of the attending physician. The compositions, molecules and cells are in some embodiments suitably administered to the patient at one time or over a series of treatments. Multiple doses may be administered intermittently. An initial higher loading dose, followed by one or more lower doses may be administered.

In the context of genetically engineered cells, in some embodiments, a subject may be administered the range of about one million to about 100 billion cells and/or that amount of cells per kilogram of body weight. In some embodiments, wherein the pharmaceutical composition comprises any one of the engineered immune cells described herein, the pharmaceutical composition is administered at a dosage of at least about any of 10⁴, 10⁵, 10⁶, 10⁷, 10⁸, or 10⁹ cells/kg of body weight of the individual. Dosages may vary depending on attributes particular to the disease or disorder and/or patient and/or other treatments.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once or multiple times during a dosing cycle. A dosing cycle can be, e.g., 1, 2, 3, 4, 5 or more week(s), or 1, 2, 3, 4, 5, or more month(s). The optimal dosage and treatment regime for a particular patient can be determined by one skilled in the art of medicine by monitoring the patient for signs of disease and adjusting the treatment accordingly.

In some embodiments, the compositions provided herein are administered as part of a combination treatment, such as simultaneously with or sequentially with, in any order, another therapeutic intervention, such as another antibody or engineered cell or receptor or agent, such as a cytotoxic or therapeutic agent.

In some embodiments, the compositions provided herein are co-administered with one or more additional therapeutic agents or in connection with another therapeutic intervention, either simultaneously or sequentially in any order. In some embodiments, the cells are co-administered with another therapy sufficiently close in time such that the cell populations enhance the effect of one or more additional therapeutic agents, or vice versa. In some embodiments, the compositions provided herein are administered prior to the one or more additional therapeutic agents. In some embodiments, the compositions provided herein are administered after to the one or more additional therapeutic agents.

In certain embodiments, once the cells are administered to a mammal (e.g., a human), the biological activity of the engineered cell populations is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load.

### 7.9. Kits and Articles of Manufacture

Further provided are kits, unit dosages, and articles of manufacture comprising any of the engineered immune effector cells described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 8. EXAMPLES

The examples described herein are not intended to represent that the experiments below are all or the only experiments performed. Efforts have been made to ensure accuracy with respect to numbers used (for example, amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

### 8.1. Example 1—Preparation of CAR-T Cells Expressing Exogenously Introduced p40 and CCL-19

Exemplary CAR-T cells expressing exogenously introduced p40 and CCL-19 are constructed and tested. For example, in this and following examples, anti-GPC3 CAR-T cells were constructed to exemplify the present invention.

### 8.1.1. Construction of Chimeric Antigen Receptors

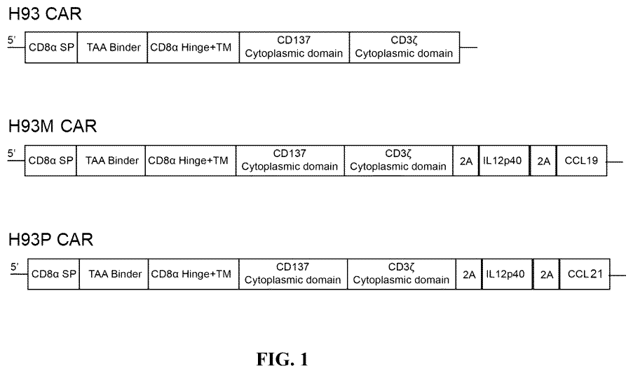

To construct humanized anti-GPC3 scFv CAR (i.e., H93 CAR), a CAR backbone sequence encoding a CAR backbone polypeptide comprising from the N-terminus to the C-terminus: a CD8α hinge domain (SEQ ID NO: 8), a CD8α transmembrane domain (SEQ ID NO: 9), a CD137 co-stimulatory signaling domain (SEQ ID NO: 10), and a CD3ζ cytoplasmic signaling domain (SEQ ID NO: 11) was chemically synthesized and cloned into a pre-modified lentiviral vector (pLSINK-BBzBB) downstream and operably linked to a constitutive hEF1α promoter for in vitro transcription. Multi-cloning sites (MCS) in the vector allowed insertion of a nucleic acid sequence comprising a Kozak sequence operably linked to a nucleic acid sequence encoding a CD8α signal peptide (SEQ ID NO: 7) fused to the N-terminus of an anti-GPC3 humanized scFv fragment (i.e., a scFv comprising the amino acid sequence of SEQ ID NO: 1) into the CAR backbone vector, upstream and operably linked to the CAR backbone sequence. The nucleic acid sequence encoding the CD8α signal peptide and the anti-GPC3 humanized scFv fragment was chemically synthesized and cloned into pLSINK-BBzBB CAR backbone via the EcoRI (5′-GAATTC-3′) (SEQ ID NO: 15) and SpeI (5′-ACTAGT-3′) (SEQ ID NO: 16) restriction sites by molecular cloning techniques known in the art. The resulting H93 CAR comprises the amino acid sequence of SEQ ID NO: 2. The CAR coding region is presented in the top part of FIG. 1.

Based on H93 CAR backbone, MCS in H93M CAR vector allowed insertion of a nucleic acid sequence comprising a nucleic acid sequence encoding human IL12p40 (SEQ ID NO: 5) and human CCL-19 (SEQ ID NO: 6) linked by a 2A self-cleaving peptide (e.g., a T2A peptide shown in SEQ ID NO: 14) fused to the C-terminus of another 2A self-cleaving peptide (e.g., a P2A fragment shown in SEQ ID NO: 13), upstream and operably linked to the C-terminus of CD3ζ cytoplasmic signaling domain. The nucleic acid sequence encoding the P2A peptide and IL12p40-T2A-CCL-19 peptide was chemically synthesized and cloned into pLSINK-BBzBB CAR backbone via the HpaI (5′-GTTAAC-3′) (SEQ ID NO: 17) and MluI (5′-ACGCGT-3′) (SEQ ID NO: 23) restriction sites. The resulting H93M CAR comprises the amino acid sequence of SEQ ID NO: 3, and the nucleic acid sequence of SEQ ID NO: 12. The CAR coding region of H93M is presented in the middle part of FIG. 1. Without being bound to any theory, overexpressed IL12p40 in H93M CAR-T cells could form IL-23 with the endogenous IL-23α p19 subunit which was reported to being upregulated by T cells upon TCR stimulation. See e.g., Ma, et al., Interleukin-23 engineering improves CAR T cell function in solid tumors. Nat Biotechnol. 2020 April; 38(4):448-459.

The lentivirus packaging plasmid mixture containing pMDLg.pRRE (Addgene #12251), pRSV-REV (Addgene #12253) and pMD2.G (Addgene #12259) were pre-mixed with the vectors expressing CAR constructs at a pre-optimized ratio with polyetherimide (PEI), then incubated at 25° C. for 5 min. Then HEK293 cells were added into the transfection mix. Afterwards, cells were incubated overnight in a cell incubator with 5% CO₂ at 37° C. The supernatants were collected after centrifuged at 4° C. and 3000 g for 15 min, and filtered through a 0.45 μm PES filter followed by ultra-centrifugation for lentivirus concentration. The supernatants were carefully discarded and the virus pellets were rinsed cautiously with pre-chilled DPBS. The viruses were liquated properly, and stored at −80° C. The virus titer was determined by a titration method via transduction of CHO (Chinese hamster ovarian) cell line.

### 8.1.2. CAR-T Cell Preparation

PBMCs were purchased from TPCS (#A19Z284097). Human T cells were purified from PBMCs using Pan T cell isolation kit (Miltenyi #130-096-535), following manufacturer's protocol as described below. Cell number was determined and the cell suspension was centrifuged at 300 g for 10 minutes. The supernatant was then aspirated completely, and the cell pellets were re-suspended in 40 μL buffer per 10⁷ total cells. 10 μL of Pan T Cell Biotin-Antibody Cocktail was added per 10⁷ total cells, mixed thoroughly and incubated for about 5 minutes in the refrigerator (2-8° C.). 30 μL of buffer was then added per 10⁷ cells. 20 μL of Pan T Cell MicroBead Cocktail was added per 10⁷ cells. The cell suspension mixture was mixed well and incubated for an additional 10 minutes in the refrigerator (2-8° C.). A minimum of 500 μL was required for magnetic separation. For magnetic separation, an LS column was placed in the magnetic field of a suitable MACS Separator. The column was prepared by rinsing with 3 mL of buffer. The cell suspension was then applied onto the column, and flow-through containing the unlabeled cells was collected, which represented the enriched T cell fractions. Additional T cells were collected by washing the column with 3 mL of buffer and collecting unlabeled cells that pass through. These unlabeled cells again represented the enriched T cells, and were combined with the flow-through from previous step. The pooled enriched T cells were then centrifuged and re-suspended in RPMI-1640+300 IU/mL IL-2.

The prepared T cells were subsequently pre-activated for 48 hours with human T cell TransAct kit (Miltenyi #170-076-156) according to manufacturer's protocol in which anti-CD3/CD28 MACSiBead particles were added at a bead-to-cell ratio of 40 μL/million.

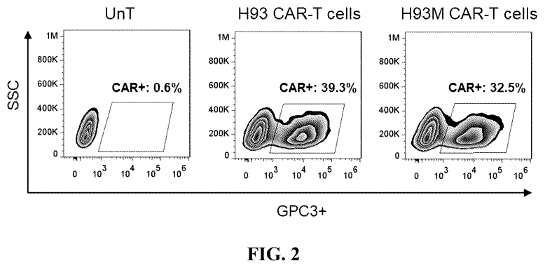

For CAR-T cells preparation, the pre-activated T cells were plated in 24-well plates at a density of 0.5×10⁶ cells/well. Then, the purified virus were added into the wells. After overnight, the transduced cells were placed into a 24-well G-Rex (Wilson Wolf #80192M) after collecting, centrifugation and re-suspended with fresh RPMI-1640+300 IU/mL IL-2. Un-transduced T cells (UnT) were used as a negative control. On day 7, the CAR positive rates of naked H93 CAR-T cells and H93M CAR-T cells were 39.3% and 32.5%, respectively (FIG. 2).

### 8.1.3. IL-23 and CCL-19 Expression

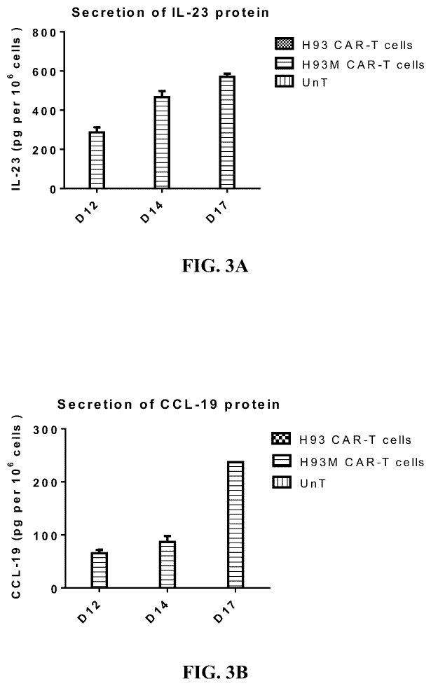

In order to verify the structure of H93M CAR, the CAR-T cells were cultured in normal condition (RPMI-1640+300 IU/mL IL-2) and the secretion of IL-23 and CCL-19 were analyzed. Briefly, cells were cultured at 1×10⁶ cells/mL in 6-well plates and supernatants were harvested on day 12, 14 and 17, respectively. The human IL-23 kit (CISBIO #62HIL23PEG) and CCL-19 ELISA kit (Abcam #ab100601) were used to determine the amount of IL-23 and CCL-19 according to manufacturer's protocol. As shown in FIG. 3, only H93M CAR-T cells had the secretion of IL-23 (286.9 pg per 10⁶ cells-570.3 pg per 10⁶ cells) and CCL-19 (65.4 pg per 10⁶ cells-237.2 pg per 10⁶ cells) compared with H93 CAR-T cells and UnT (Neither IL-23 nor CCL-19 expression was detected).

### 8.2. Example 2—In Vitro Repeat Challenge Assay for CAR-T Cells Expressing Exogenously Introduced p40 and CCL-19

To evaluate the persistence and exhaustion of CAR-T cells in vitro, CAR-T cells repeat challenge model was set up. CAR-T cells were constantly stimulated by GPC3 positive tumor cells for several rounds to gain an exhaustion phenotype of CAR-T cells. There were three groups of treatment in the re-challenge assay: H93 CAR-T group (the initial CAR-T cells were H93 CAR-T cells), H93M CAR-T group (the initial CAR-T cells were H93M CAR-T cells) and UnT (the initial T cells were un-transduced with CAR). For the first round (round 1) of re-challenge, CAR-T cells were co-cultured with tumor cells at 1:1 E/T ratio in a 6-well plate. After overnight, CAR-T cells in the well were gently collected, centrifuged, and re-suspended in fresh CAR-T culture medium (RPMI-1640+300 IU/mL IL-2). Then CAR-T cells were added to a new plate for another 2 days. Where after, CAR-T cells in the well were collected, re-suspended in fresh medium again and added to a new plate seeded with fresh tumor cells (round 2). The number of fresh tumor cells added was calculated by the positive ratio of CAR-T cells at the end of the former round. This procedure could be repeated for several rounds depending on the cell number and viability of the remaining CAR-T cells. Exhaustion markers PD-1 and LAG3 of CD4+ T cells and CD8+ T cells in CAR+ T cells were analyzed 24 hours after each round's tumor cell re-challenge. T cells were counted at the end of each round and the cytotoxicity assay was performed on GPC3 positive HCC cell line PLC/PRF/5 72 hours after stimulation.

### 8.2.1. Expansion of T Cells

To determine the benefits of the expansion function of H93M CAR-T cells, the cell number and viability of total T cells were recorded at the end of each round of re-challenge.

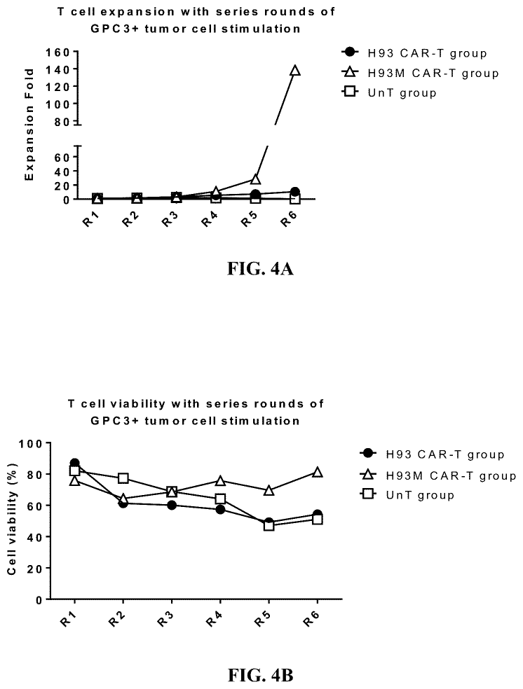

As shown in FIG. 4A, repeat stimulations with PLC/PRF/5 cells resulted in a significant expansion of cells in H93M CAR-T group (138.4 folds at round 6) in comparison with that in H93 CAR-T group (10.5 folds at round 6) and that in UnT group (0.2 folds at round 6). In parallel, the cell viability of H93M CAR-T group was remained high, as a comparison, the H93 CAR-T group and UnT group were gradually reduced after each stimulation (FIG. 4B).

### 8.2.2. Cytotoxicity Assay and Cytokine Release

The cytotoxicity of CAR-T cells was also assessed at the end of every two rounds in CAR-T re-challenge assay at total T cells:target cells ratio of 1:1. The target cell was GPC3 positive PLC/PRF/5 cell, which was engineered to express firefly luciferase with the techniques known in the art. The cells collected at the end of every two rounds and the target cells were mixed (2×10³ cells/well each), cultured in 384-well plates for 24 hours. To assay the cytotoxicity of CAR-T cells on tumor cells, One-glo luminescent luciferase assay reagents (Promega #E6110) were prepared according to manufacturer's protocol and added to the co-cultured cells to detect the remaining luciferase activity in the well. Since luciferase is expressed only in tumor cells, the remaining luciferase activity in the well correlates directly to the number of viable target cells in the well. The maximum luciferase activity was obtained by adding culture media to target cells in the absence of effector cells. The minimum luciferase activity was determined by adding Triton X-100 at a final concentration of 1% at the time when the cytotoxicity assays were initiated. The specific cytotoxicity was calculated by the formula: Specific Cytotoxicity %=100%×(1−(RLUsample−RLUmin)/(RLUmax−RLUmin)).

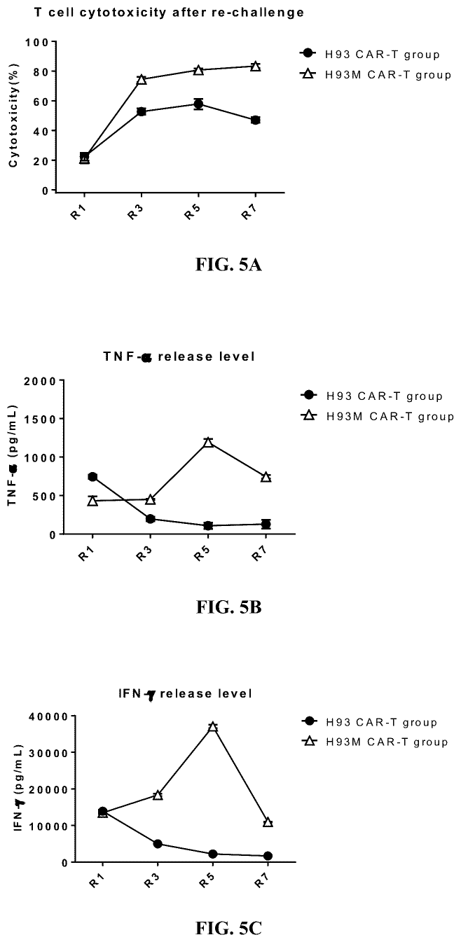

As shown in FIG. 5A, H93M CAR-T group showed higher cytotoxicity than H93 CAR-T group after round 3, except that after the round 1 showed equal cytotoxicity (21.1% vs 22.4%). Concretely, H93M CAR-T group exhibited average cytotoxicity of 74.4% after round 3, and increased slightly after round 5 (80.7%) and round 7 (83.4%). While the relative lower cytotoxicity was observed in H93 CAR-T group, which showed average cytotoxicity of 52.7% (round 3), 57.9% (round 5) and 47.0% (round 7), respectively. Indicating H93M CAR-T group had a sustained high levels of cytotoxicity on GPC3 positive-HCC cell line PLC/PRF/5.Luc cells.

To further verify the sustained cytotoxic activity of H93M CAR-T cells against GPC3 positive tumor cells in comparison with H93 CAR-T cells in the re-challenge assay, TNF-α and IFN-γ production were analyzed by ELISA after coculturing CAR-T cells with PLC/PRF/5.Luc cells at 1:1 E/T ratio (total T cells:target cells) for 24 hours. As a result, the ability to produce TNF-α and IFN-γ of H93M CAR-T group were significant higher compared with H93 CAR-T group, and showed significant correlation with cytotoxicity potency in all rounds. As shown in FIGS. 5B and 5C, the concentration of TNF-α and IFN-γ secreted by cells in H93M CAR-T group significantly increased after round 3 to round 5, within the range of 451.2-1151.1 pg/mL (TNF-α) and 18353.7-37072.7 pg/mL (IFN-γ), while the concentration of TNF-α and IFN-γ secreted by cells in H93 CAR-T group were gradually reduced after round 3 to round 7, within the range of 197.1-129.2 pg/mL (TNF-α) and 4993.1-1672.1 pg/mL (IFN-γ).

### 8.2.3. Rates Change of Positive CAR-T Cells

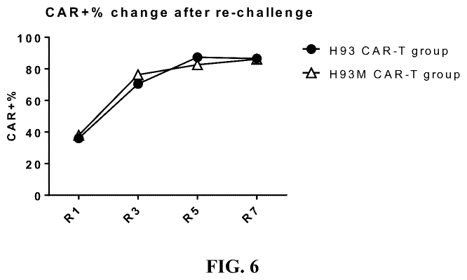

The positive rates of H93 CAR-T cells and H93M CAR-T cells were calculated after each two rounds, which were almost identical increasing with the rounds of stimulation, within the range of 30%-86%. Finally, the positive rates of H93 CAR-T cells and H93M CAR-T cells were not increased after round 5 (FIG. 6). H93M CAR-T cells showed high cytotoxicity comparing the increasing cytotoxicity (FIG. 5A) with the flat CAR+% (FIG. 6) after round 5 re-challenge.

### 8.2.4. Exhaustion of T Cells

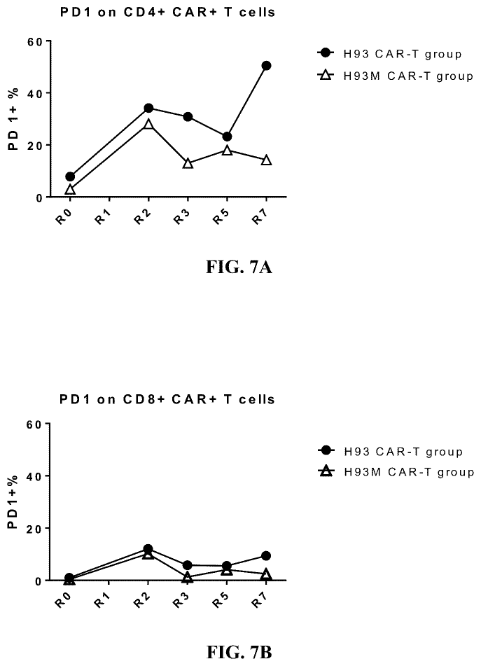

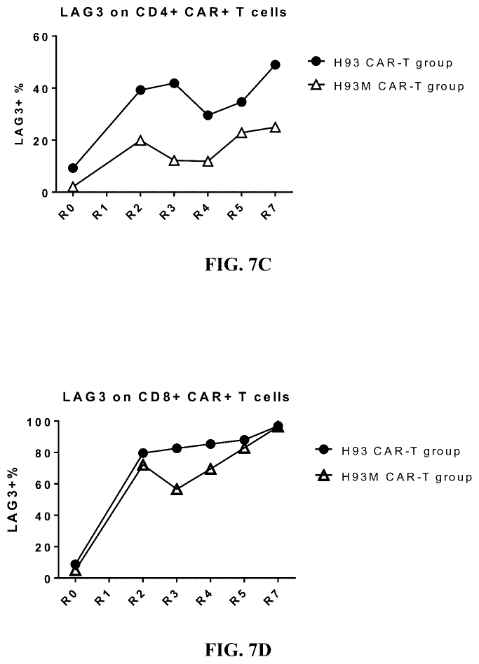

T cell exhaustion was evaluated 24 hours after each round in the re-challenge assay. PD-1 and LAG-3 as two exhaustion markers were marked using anti-human PD-1 antibody (Biolegend #329908) and anti-LAG-3 antibody (Invitrogen #17-2239-42), respectively and the expression levels of CD4+CAR+ T cells and CD8+CAR+ T cells double positive subsets were analyzed. As shown in FIG. 7, H93M CAR-T group showed reduced expression of the exhaustion markers PD-1 and LAG3, as compared to H93 CAR-T group in all rounds. Especially in rounds 3 to 5, the PD-1 expression of CD4+CAR+ double positive subset in H93M CAR-T group was between 13% and 18%, while H93 CAR-T group was above 30% (FIG. 7A). H93M CAR-T group had significant lower LAG-3 expression (12%-25%) than H93 CAR-T group (30%-49%) in CD4+CAR+ double positive subset in all rounds (R2-R7) (FIG. 7C). In CD8+CAR+ double positive subset, the expression level of PD-1 in H93M CAR-T group was still lower than H93 CAR-T group (1.3%-4.1% vs 5.6%-9.4% in round 3 to round 7) (FIG. 7B), and the LAG3 expression level of H93M CAR-T group was between 56.6% and 82.9% that was lower than that of the H93 CAR-T group (82.7%-88.1%) in round 3 to round 5 (FIG. 7D), indicating H93M CAR-T cells have the ability of resistance to exhaustion.

### 8.3. Example 3—Cell Migration Assay for CAR-T Cells Expressing Exogenously Introduced p40 and CCL-19

To evaluate the chemotactic function of CCL-19, cell migration assay was performed. Chemotaxis of the responder T cells was measured by migration through a polycarbonate filter of 5-μm pore-size in 96-well transwell chambers (Corning). CAR-T cells and UnT cells were stimulated with GPC3 positive human HCC cell line PLC/PRF/5 cells and the co-culture supernatant was collected after 30 hours. Then 125 μL of supernatant was placed in the lower chambers, and 75 μL untreated T cells (0.075 M) were incubated in the upper chambers. After 4, 6 and 8 hours, the T cells migrated from the upper chamber to the lower chamber were counted by blood counting chamber. There were three groups of treatment in cell migration assay: H93 CAR-T group (the supernatant in the lower chamber was from H93 CAR-T cells co-cultured with PLC/PRF/5 cells), H93M CAR-T group (the supernatant in the lower chamber was from H93M CAR-T cells co-cultured with PLC/PRF/5 cells) and UnT group (the supernatant in the lower chamber was from UnT cells co-cultured with PLC/PRF/5 cells).

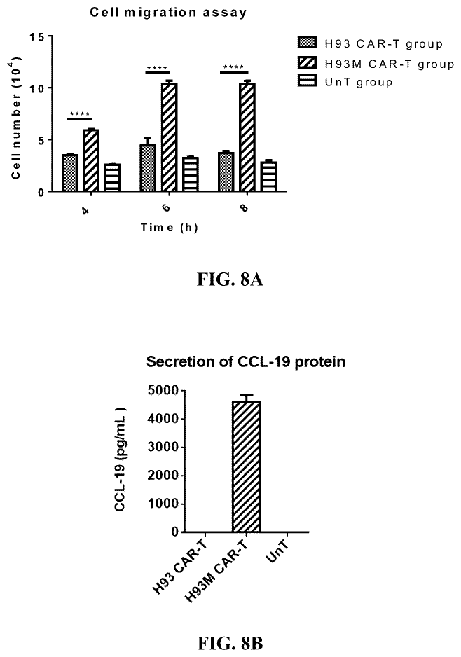

As shown in FIG. 8A, more T cells were attracted into the supernatant of H93M CAR-T cells (with secretion of CCL-19) co-cultured with PLC/PRF/5 cells than that of H93 CAR-T cells co-cultured with PLC/PRF/5 cells and that of UnT cells co-cultured with PLC/PRF/5 cells at different timing (H93M CAR-T group vs H93 CAR-T group: P<0.0001). Concretely, H93M CAR-T group could completely attract all T cells of upper chambers on 6 hours (0.104 M), while H93 CAR-T group and UnT group with no production of CCL-19 only migrated fewer T cells even on 8 hours, 0.032 M and 0.026 M respectively. In addition, the concentration of CCL-19 in co-culture supernatant was measured by CCL-19 ELISA (Abcam #ab100601). This result indicated that the supernatant from H93M CAR-T cells co-cultured with PLC/PRF/5 cells had secretion of CCL-19 (4590.15±380.85 pg/mL) that further verify the chemotactic function of H93M CAR-T cells (FIG. 8B).

### 8.4. Example 4—Anti-Tumor Effect of CAR-T Cells Expressing Exogenously Introduced p40 and CCL-19 after Adoptive Transfer In Vivo

Following the methods disclosed in Example 1, H93C CAR-T cells expressing human IL12p40 and human IL-7 were also prepared. Based on H93 CAR backbone, MCS in H93C CAR vector allowed insertion of a nucleic acid sequence comprising a nucleic acid sequence encoding human IL 12p40 (SEQ ID NO: 5) and human IL-7 (SEQ ID NO: 24) linked by a 2A self-cleaving peptide (e.g., a P2A peptide shown in SEQ ID NO: 13) fused to the C-terminus of another 2A self-cleaving peptide (e.g., a P2A fragment shown in SEQ ID NO: 13), upstream and operably linked to the C-terminus of CD3ζ cytoplasmic signaling domain. The nucleic acid sequence encoding the P2A peptide and IL 12p40-P2A-IL-7 peptide was chemically synthesized and cloned into pLSINK-BBzBB CAR backbone via the HpaI (5′-GTTAAC-3′) (SEQ ID NO: 17) and MluI (5′-ACGCGT-3′) (SEQ ID NO: 23) restriction sites. The amino acid sequence and nucleic acid sequence of IL 12p40-P2A-IL-7 peptide are shown in SEQ ID NO: 25 and SEQ ID NO: 26.

In vivo anti-tumor efficacy of CAR-T cells were evaluated in NCG mouse xenograft model. For the subcutaneous xenograft model, NCG mice aged 6-7 weeks were inoculated with Huh7 cells subcutaneously in the right foreleg (1.5×10⁶ cells/mouse). When the average tumor volume approached to 100 mm³ (day 11 after the xenograft inoculation), mice were randomized into several groups and treated with H93 CAR-T cells (at 0.2 M or 0.6 M dosage), H93M CAR-T cells (at 0.2 M or 0.6 M dosage), H93C CAR-T cells (at 0.2 M dosage), UnT (at 2.3 M dosage, same cell number of total T cells with CAR-T groups) and solvent only (Vehicle), respectively, by tail vein injection. Tumor size was measured with digital calipers twice per week. Tumor volume was calculated according to the following formula: Tumor volume=((length)×(width)²)/2. The copy number of CAR-T cells in mouse blood were also detected by digital PCR on the day 0, 7, 14, 21 and 28 after the adoptive transfer, respectively.

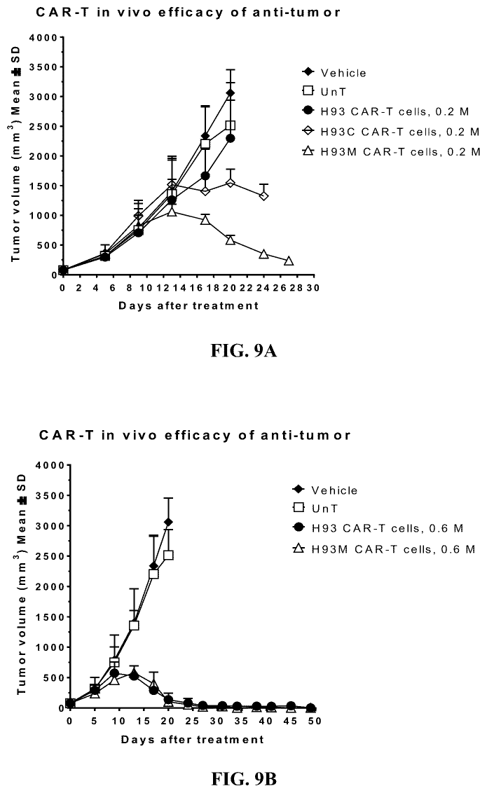

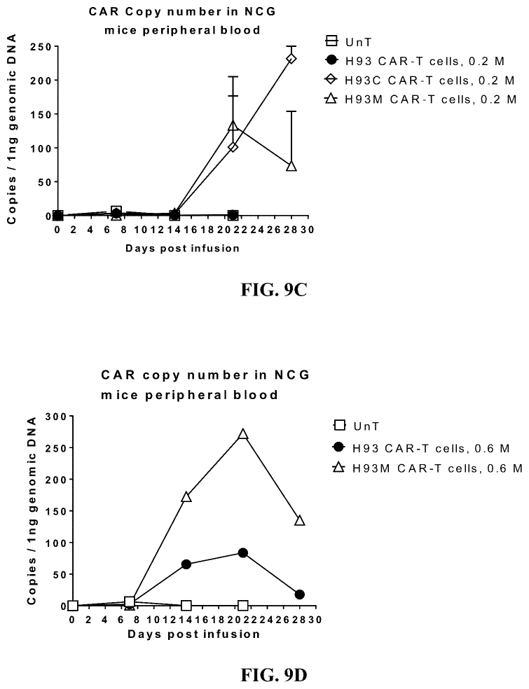

Mice treated with Vehicle, UnT and H93 CAR-T cells were sacrificed on day 20 because of animal ethics and the tumor volume of these three groups had reached more than 2000 mm³. Mice treated with H93M CAR-T cells were continued to record until day 27 (dosage: 0.2 M) and day 49 (dosage: 0.6 M). As shown in FIG. 9A, compared to the UnT and H93 CAR-T cells groups, mice administered with adoptive transfer of H93M CAR-T cells were observed to have a 76.8% and 74.6% reduction respectively in tumor size (584.3 mm³ vs 2514.7 mm³, 2297.3 mm³) at day 20, and after day 20, the tumor size still gradually reduced, which suggested H93M CAR-T cells had better therapeutic potential than H93 CAR-T cells at lower dosage (0.2 M/mouse). Meanwhile, H93C CAR-T cells group only had slight antitumor effect and had a growth inhibition rate of 32.7% at day 20 (1546.8 mm3 vs 2297.3 mm3), which suggested H93M CAR-T cells expressing exogenously introduced IL12p40 and CCL-19 had better therapeutic potential than H93C CAR-T cells expressing exogenously introduced IL 12p40 and IL-7 after adoptive transfer in vivo. When the dosage was increased to 0.6 M/mouse, as shown in FIG. 9B, administration of H93M CAR-T cells had an obvious inhibitory effect on tumor growth with a growth inhibition rate of 96.0% in comparison with UnT (101.6 mm³ vs 2514.7 mm³) at day 20, but similar tumor regression was observed in the H93M CAR-T cells treatment group and H93 CAR-T cells treatment group (101.6 mm³ vs 137.3 mm³). On day 34, the mice treated with H93M CAR-T cells had almost tumor free.

CAR gene copy number in mouse peripheral blood cells was detected by digital PCR, and H93M CAR-T cells exhibited significant expansion on day 21 and reduced subsequently at 0.2 M dosage, while there was no obvious expansion in the mice treated with H93 CAR-T cells and UnT throughout the treatment (FIG. 9C). At 0.2 M dosage, the copy number of H93M CAR-T cells at day 21 was 335.6 folds than that at day 0 (133.4175 copies vs 0.3975 copies) and decreased to 184.7 folds at day 28 (73.4250 copies vs 0.3975 copies). H93C CAR-T cells exhibited uncontrollable expansion after day 14, and reached 4873.7 folds (231.5350 copies vs 0.0475 copies) at day 28 (FIG. 9C). H93M CAR-T cells and H93 CAR-T cells both showed significant expansion after day 7 at 0.6 M dosage, and H93M CAR-T cells had a better ability of expansion than H93 CAR-T cells (272.2850 copies vs 83.6275 copies on day 21) (FIG. 9D).

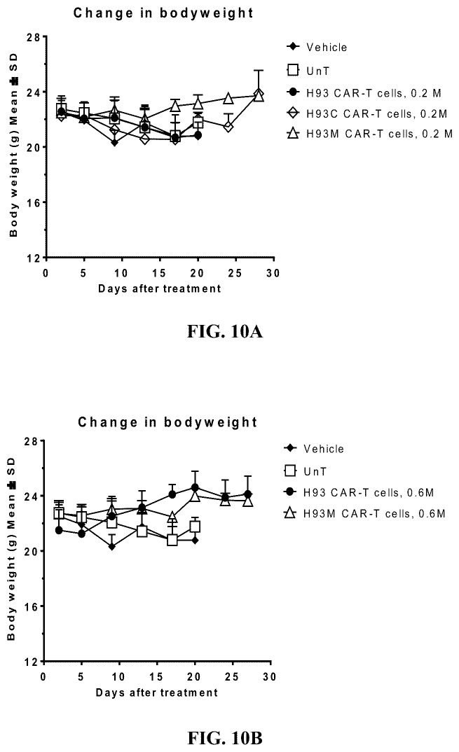

No mouse bodyweight loss was observed in all the three groups whether 0.2 M dosage or 0.6 M dosage during this experiment (FIGS. 10A and 10B). But the mice treated with H93 CAR-T cells showed such symptoms as bristling, arched back and emaciation on day 20 at 0.2 M dosage, while the mice treated with H93M CAR-T cells were normal. At 0.6 M dosage, the mice of both groups had normal appearance. 2 of the 4 mice administered with the adoptive transfer of H93C CAR-T cells (at 0.2 M dosage) were sacrificed on day 24 and showed such symptoms as bristling, arched back, emaciation and shortness of breath. The data comparison of mice treated with H93M CAR-T cells and H93C CAR-T cells indicated that H93M CAR-T cells have better antitumor potential, controllable expansion and safety in vivo than H93C CAR-T cells.

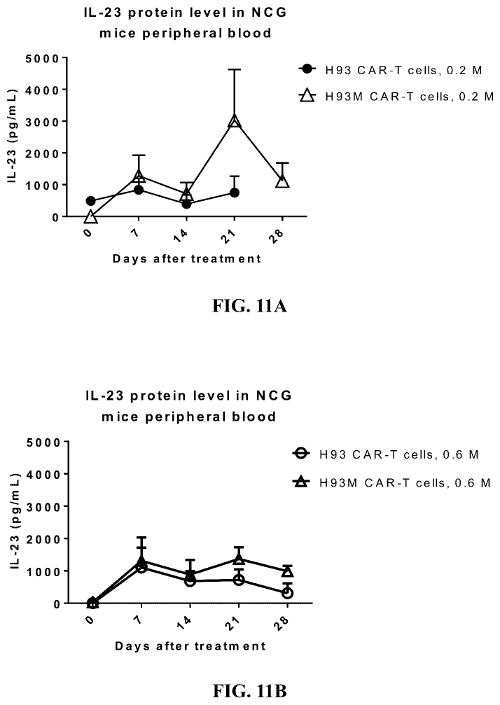

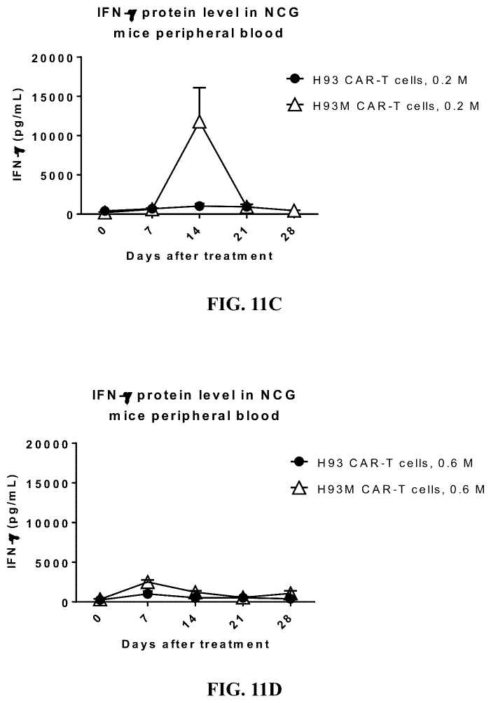

In order to verify the activation-dependent production of IL-23 in H93M CAR-T cells, the secretion level of IL-23 and IFN-γ in NCG mice peripheral blood after being treated with CAR-T cells were analyzed by using human IL-23 kits (CISBIO #62HIL23PEG) and human IFN-γ kits (CISBIO #62HIFNGPEG). As shown in FIG. 11A, H93M CAR-T cells treatment group showed up to 3 folds higher secretion level of IL-23 at low dose (0.2 M, day 21), compared with H93 CAR-T cells treatment group. Similar low level of IL-23 in H93M CAR-T cells treated mice and H93 CAR-T cells treated mice at high dose (0.6 M) was observed (FIG. 11B). In parallel, the secretory behavior of IFN-γ that was activation-dependent production in mice peripheral blood were similar with IL-23 (FIGS. 11C and 11D), indicating the activation-dependent production of IL-23 in H93M CAR-T cells.

To investigate the anti-tumor effects of H93M CAR-T cells in vivo, immunohistochemistry (IHC) was used to study the infiltration of T cells and the recruitment of macrophages and dendritic cells (DCs) in the tumor site. In brief, the tissue paraffin section slides were heated at 60° C. for 60 minutes, and then dewaxed three times in xylene for 10 minutes each and rehydrated in 100%, 100%, 95%, 90%, 80%, 70% alcohols and water for 5 minutes each. The antigen retrieval was performed by boiling the slides in the antigen retrieval buffer for 20 minutes, and the slides were then treated with 3% hydrogen peroxide in methanol for 10 minutes to deprive the endogenous peroxidase activity after the slides were immersed in the antigen retrieval buffer and cooled to Room Temperature (RT). To reduce nonspecific background staining, the slides were incubated with blocking buffers (5% goat serum diluted with DPBS) at 2-8° C. overnight.

After overnight, the slides were incubated at RT for 30 minutes, and then incubated with diluted primary antibody at 37° C. for 2 hours and second antibody HRP conjugated anti-mouse IgG antibody (Fuzhou Maixin Biotech. Co., Ltd. #KIT-5002) at RT for 15 minutes in the dark. The DAB substrate was prepared according to the Manufacturer's instructions, and 200-500 μL DAB substrate was added to the slides and incubate for 2 minutes in the dark. The staining was stopped by washing with distilled water for 5 minutes. Finally, the slides were added neutral balsam and mounted after counterstained with hematoxylin, differentiated in 1% hydrochloric acid alcohol and dehydrated in alcohols.

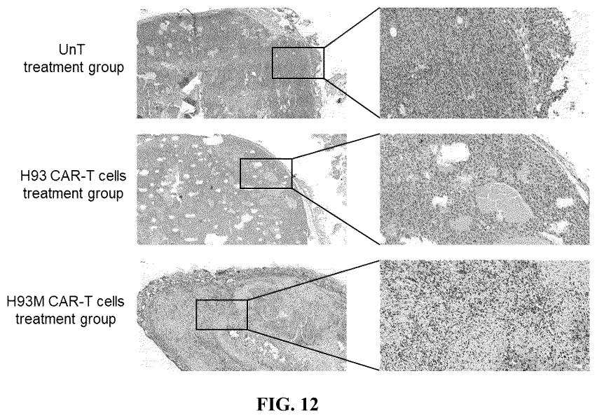

To elucidate the mechanisms of enhanced antitumor ability of H93M CAR-T cells at a lower dosage (0.2 M), T cell infiltration was investigated in tumor tissues of treated mice by IHC using TCRα (H-1) (SANTA CRUZ BIOTECHNOLOGY #sc-515719). As shown in FIG. 12, there was improved T cell infiltration into tumor sites of all three mice in the H93M CAR-T cells treatment group compared to the H93 CAR-T cells and UnT treatment groups. More T cells infiltrated into the central tumor nest in H93M CAR-T cells treatment group, while only one mouse among three H93 CAR-T cells treated mice had fewer T cell infiltration in the tumor marginal region and other mice had very few T cells in the tumor site. In UnT treatment group, there was no T cell infiltration observed in tumor site among three mice.

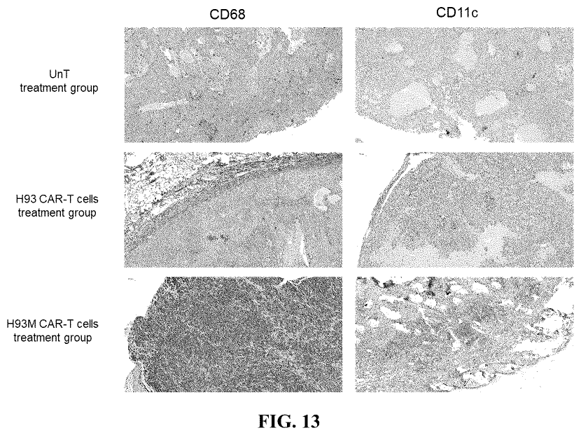

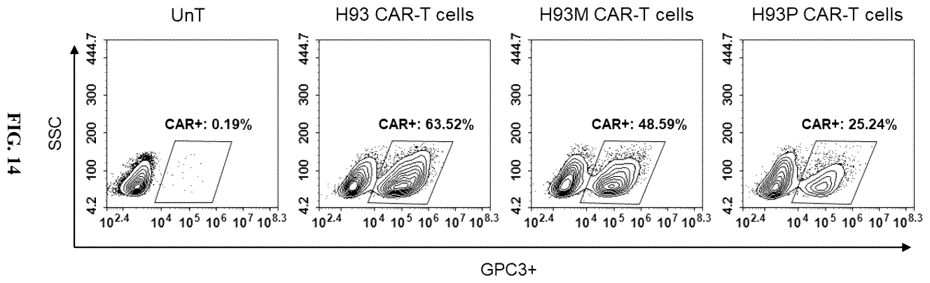

In addition, macrophage and DC recruitment in tumor tissues were investigated. Anti-CD68 antibody (Abcam #ab125212) and Anti-CD11c antibody (Cell signaling technology #97585S) were used to detect mouse macrophages and DCs in tumor tissues. As shown in FIG. 13, CD68+ macrophages (left column) and CD11c+ DCs (right column) were stained by DAB. Macrophage and DC recruitment to the tumor site were significantly higher in the H93M CAR-T cells treatment group, while the UnT and H93 CAR-T cells treatment groups only had macrophage recruitment in a margin of tumor tissues. These data reveal that H93M CAR-T cells can induce more macrophage and DC recruitment in the tumor tissues compared to H93 CAR-T cells and UnT, and are more potent to elicit innate immune responses in tumor tissue.

### 8.5. Example 5—Preparation of CAR-T Cells Expressing Exogenously Introduced p40 and CCL-21

PBMCs were purchased from TPCS (#A19K268037). Human T cells were purified from PBMCs using Pan T cell isolation kit (Miltenyi #130-096-535), following manufacturer's protocol as described in Section 8.1.2 above.

The prepared T cells were subsequently pre-activated for 48 hours with human T cell TransAct kit (Miltenyi #170-076-156) according to manufacturer's protocol in which anti-CD3/CD28 MACSiBead particles were added at a bead-to-cell ratio of 40 μL/million. For CAR-T cells preparation, the pre-activated T cells were plated in 24-well plates (BD) at a density of 0.5×10⁶ cells/well. Then, the purified virus were added into the wells. After overnight, the transduced cells were placed into a 24-well G-Rex (Wilson Wolf #80192M) after collecting, centrifugation and re-suspended with fresh RPMI-1640+300 IU/mL IL-2. Un-transduced T cells (UnT) were used as a negative control. H93 CAR-T cells and H93M CAR-T cells were prepared as described above, as well as H93P CAR-T cells with H93P CAR whose coding region is presented in the bottom part of FIG. 1. H93P CAR comprises the amino acid sequence of SEQ ID NO: 18, and the nucleic acid sequence of SEQ ID NO: 19. On day 6, the CAR positive rates of naked H93 CAR-T cells, armored H93M CAR-T cells (secretion of IL-23 and CCL-19) and armored H93P CAR-T cells (secretion of IL-23 and CCL-21) were 63.52%, 48.59%, and 25.24%, respectively (FIG. 14).

### 8.6. Example 6—Re-Challenge Assay for CAR-T Cells Expressing Exogenously Introduced p40 and CCL-21 and CAR-T Cells Expressing Exogenously Introduced p40 and CCL-19

CAR-T cells repeat challenge model was set up as described in Example 2 above. GPC3 positive cell line PLC/PRF/5 cells were used as the target cell. For the first round (round 1) of re-challenge, CAR-T cells were co-cultured with tumor cells at 1:1 E/T ratio in a 6-well plate. After overnight, CAR-T cells in the well were gently collected, centrifuged, and re-suspended in fresh CAR-T culture medium (RPMI-1640+300 IU/mL IL-2). Then CAR-T cells were added to a new plate for another 2 days. Where after, CAR-T cells in the well were collected, re-suspended in fresh medium again and added to a new plate seeded with fresh tumor cells (round 2). CAR-T cells were stimulated for 8 rounds in total. T cells were counted every round and the cytotoxicity assays were performed by using GPC3 positive human hepatocellular carcinoma (HCC) cell line PLC/PRF/5.Luc 72 hours after each stimulation. Cytotoxicity assays were performed using total T cells:target cells ratio. There were four groups of treatment in re-challenge assay: H93 group (the initial CAR-T cells were H93 CAR-T cells), H93M group (the initial CAR-T cells were H93M CAR-T cells), H93P group (the initial CAR-T cells were H93P CAR-T cells) and UnT (the initial T cells were T cells un-transduced with CAR).

### 8.6.1. Expansion of T Cells

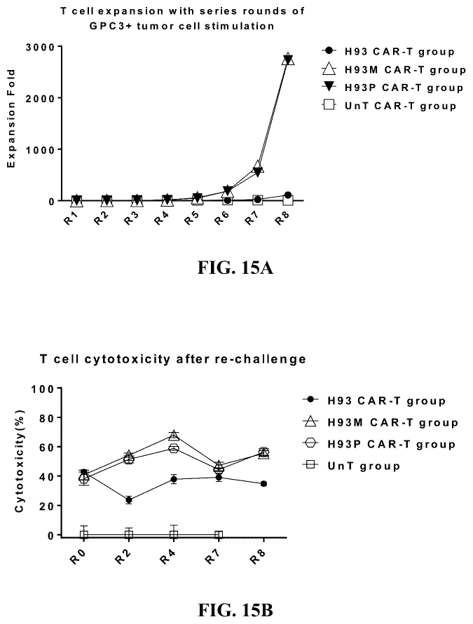

To determine the benefits of the expansion function of CAR-T cells with IL 12p40 expression in the re-challenge assay, the number of T cells were recorded at the end of each round and the expansion folds were calculated. As shown in FIG. 15A, repeat stimulations with PLC/PRF/5 cells resulted in a significant expansion of H93M CAR-T group (2759 folds at round 8) in comparison with H93 CAR-T group (109 folds at round 8) and UnT group (7 folds at round 8), and H93P CAR-T group had a similar strong expansion ability with H93M group.

### 8.6.2. Cytotoxicity Assay

The cytotoxicity of CAR-T cells was also assessed in re-challenge assay at total T cells: target cells ratio of 1:2. Target cell was GPC3 positive human hepatocellular carcinoma (HCC) cell line PLC/PRF/5.Luc that was engineered to express firefly luciferase with the techniques known in the art. Cells collected at the end of every two rounds and target cells were mixed (2×10³ cells/well), cultured in 384-well plates for 24 hours. To assay the cytotoxicity of CAR-T on tumor cells, One-glo luminescent luciferase assay reagents (Promega #E6110) were prepared according to manufacturer's protocol and added to the co-cultured cells to detect the remaining luciferase activity in the well. The specific cytotoxicity was calculated by the formula: Specific Cytotoxicity %=100%×(1−(RLUsample−RLUmin)/(RLUmax−RLUmin)). As shown in FIG. 15B, H93 CAR-T, H93M CAR-T and H93P CAR-T groups had a similar cytotoxicity at R0 (before stimulation of PLC/PRF/5). After repeat stimulation of PLC/PEF/5, H93M CAR-T group and H93P CAR-T group had similar cytotoxicity (47%-68% in all rounds), and significant higher than H93 CAR-T group (24%-39% in all rounds), indicating both H93M CAR-T and H93P CAR-T groups had a sustained high levels of cytotoxicity on GPC3 positive-HCC cell line PLC/PRF/5.Luc cells.

### 8.7. Example 7—Cell Migration Assay for CAR-T Cells Expressing Exogenously Introduced p40 and CCL-21 and CAR-T Cells Expressing Exogenously Introduced p40 and CCL-19

To evaluate the chemotactic function of CCL-19 and CCL-21, cell migration assay was performed. Chemotaxis of the responder T cells was measured by migration through a polycarbonate filter of 5-μm pore-size in 96-well transwell chambers (Corning). CAR-T and UnT cells were stimulated with GPC3 positive human HCC cell line PLC/PRF/5 cells and the co-culture supernatant was collected after 30 hours. Then 125 μL of supernatant was placed in the lower chambers, and the 75 μL untreated T cells (0.075 M) were incubated in the upper chambers. After 2, 4 and 6 hours, the T cells migrated from the upper chamber to the lower chamber were counted by blood counting chamber. There were four treatment groups: H93 CAR-T group (the supernatant in the lower chamber was from H93 CAR-T cells co-cultured with PLC/PRF/5 cells), H93M CAR-T group (the supernatant in the lower chamber was from H93M CAR-T cells co-cultured with PLC/PRF/5 cells), H93P CAR-T group (the supernatant in the lower chamber was from H93P CAR-T cells co-cultured with PLC/PRF/5 cells), and UnT group (the supernatant in the lower chamber was from UnT cells co-cultured with PLC/PRF/5 cells).

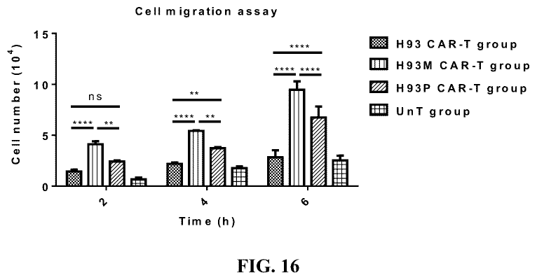

As shown in FIG. 16, more T cells were attracted into the supernatant of H93M CAR-T cells (secretion of CCL-19) and H93P (secretion of CCL-21) CAR-T cells co-cultured with PLC/PRF/5 cells than that of H93 CAR-T cells co-cultured with PLC/PRF/5 cells, and UnT cells co-cultured with PLC/PRF/5 cells at different timing. However, the H93M CAR-T group had a significant stronger ability of inducing migration in vitro than H93P CAR-T group (H93M CAR-T group vs H93P CAR-T group, P<0.0001 at 6 hours). Concretely, H93M CAR-T group could completely attract all T cells of upper chambers on 6 hours (0.095 M), and H93P CAR-T group could attract most T cells (0.065 M). While H93 CAR-T group and UnT group with no production of CCL-19 or CCL-21 only migrated fewer T cells even on 6 hours, 0.028 M and 0.025 M respectively. In addition, the concentration of CCL-19 and CCL-21 in the supernatant were measured by CCL-19 ELISA (Abcam #ab100601) and CCL-21 ELISA (Thermo #EHCCL21), respectively. This result indicated that the supernatant from H93M and H93P CAR-T cells co-cultured with PLC/PRF/5 cells had secretion of CCL-19 (2292.10±85.48 pg/mL) and CCL-21 (3481.08 f 193.11 pg/mL) respectively that further verify the chemotactic function of H93M and H93P CAR-T cells.

### 8.8. Example 8—Anti-Tumor Effect of CAR-T Cells Expressing Exogenously Introduced p40 and CCL-21 and CAR-T Cells Expressing Exogenously Introduced p40 and CCL-19 after Adoptive Transfer In Vivo

In vivo anti-tumor efficacy of CAR-T cells were evaluated in NCG mouse xenograft model. For the subcutaneous xenograft model, NCG mice aged 6-7 weeks were inoculated with Hep3B cells subcutaneously in the right foreleg (4.0×10⁶ cells/mouse). When the average tumor volume approached to 150 mm³ (day 5 after the xenograft inoculation), mice were randomized into 8 groups and treated with H93 CAR-T cells (at dosage of 0.3 M and 0.8 M), H93M CAR-T cells (at dosage of 0.3 M and 0.8 M), H93P CAR-T cells (at dosage of 0.3 M and 0.8 M), UnT (at dosage of 0.2 M) and solvent only (Vehicle), respectively, by tail vein injection. Tumor size was measured with digital calipers twice per week. Tumor volume was calculated according to the following formula: Tumor volume=(length)×(width)2/2. The number of T cells in mouse blood were also detected by FACS staining anti-human CD3 (Biolegend #300316) on the day 7, 14, 21 and 28 after the adoptive transfer.

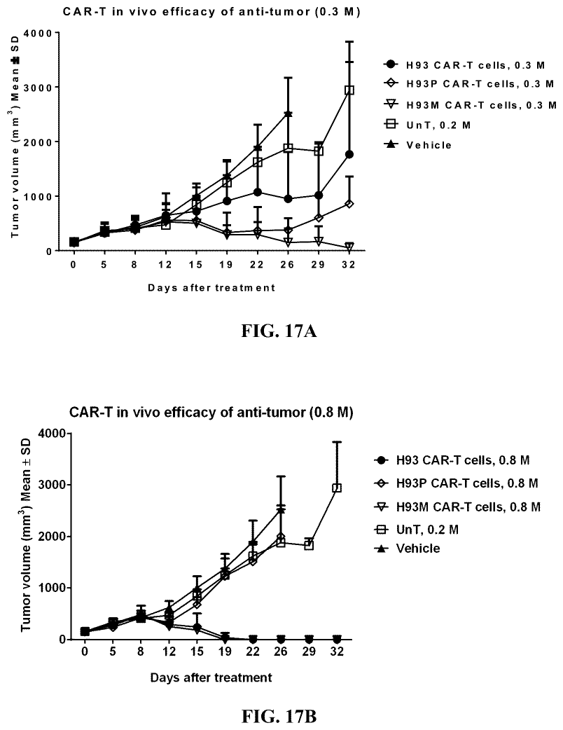

As shown in FIG. 17A, compared to the UnT and H93 CAR-T cells groups, mice administered with the adoptive transfer of H93M CAR-T cells were observed to have a 98.2% and 97.1% reduction in tumor size (52.0 mm³ vs 2942.0 mm³, 1766.5 mm³) at day 32, respectively. This result suggested H93M CAR-T cells had better therapeutic potential than H93 CAR-T cells at lower dosage (0.3 M/mouse). H93P CAR-T cells treatment group was observed similar anti-tumor efficacy with H93M CAR-T cells treatment group before day 22, and have 77.3% and 65.7% reduction respectively in tumor size (368.3 mm³ vs 1620.8 mm³, 1072.7 mm³) at day 22, but the tumor size were increased subsequently. When the dosage was increased to 0.8 M/mouse, as shown in FIG. 17B, both H93 CAR-T cells and H93M CAR-T cells had obvious inhibitory effect on tumor growth with a growth inhibition rate of 100% at day 32, but administration of H93P CAR-T cells seemed no obvious tumor volume inhibition. However, the xenograft tissues of H93P CAR-T group were observed to be spongy, different from other groups, and the mice were euthanized on day 26 because the tumor size was over 2000 mm³.

### 8.9. Example 9—Comparison of p40 and CCL-19 Armored CARs with CARs Armored with Either of them Separately

8.9.1. Comparison of p40 and CCL-19 Armored CARs with CARs Armored with Either of them Separately in NCG Mouse Xenograft Model

Based on H93 CAR backbone, MCS in H93IL12p40 CAR vector or H93CCL19 CAR vector allowed insertion of a nucleic acid sequence comprising a nucleic acid sequence encoding human IL 12p40 (SEQ ID NO: 5) or human CCL-19 (SEQ ID NO: 6) fused to the C-terminus of 2A self-cleaving peptide (e.g., a P2A fragment shown in SEQ ID NO: 13), upstream and operably linked to the C-terminus of CD3ζ cytoplasmic signaling domain.

To elucidate the synergy in H93M CAR-T cells, H93M CAR-T cells, H93IL12p40 CAR-T cells (with secretion of IL 12p40), H93CCL19 CAR-T cells (with secretion of CCL-19) were prepared and evaluated in NCG mouse xenograft model. As disclosed in Example 8, NCG mice aged 6-7 weeks were inoculated with Hep3B cells subcutaneously in the right foreleg (4.0×10⁶ cells/mouse). When the average tumor volume approached to 150 mm³ (day 5 after the xenograft inoculation), mice were randomized into 6 groups and treated with H93 CAR-T cells (at 0.3 M dosage), H93M CAR-T cells (at 0.3 M dosage), H93IL 12p40 CAR-T cells (at 0.3 M dosage), H93CCL 19 CAR-T cells (at 0.3 M dosage), UnT (at 3.5 M dosage, same cell number of total T cells with CAR-T groups) and solvent only (Vehicle), respectively, by tail vein injection. Tumor size was measured with digital calipers twice per week. Tumor volume was calculated according to the following formula: Tumor volume=((length)×(width)²)/2. The number of T cells in mouse blood were detected by FACS staining anti-human CD3 (Biolegend #300316) on the day 7, 14, 21 and 28 after the adoptive transfer.

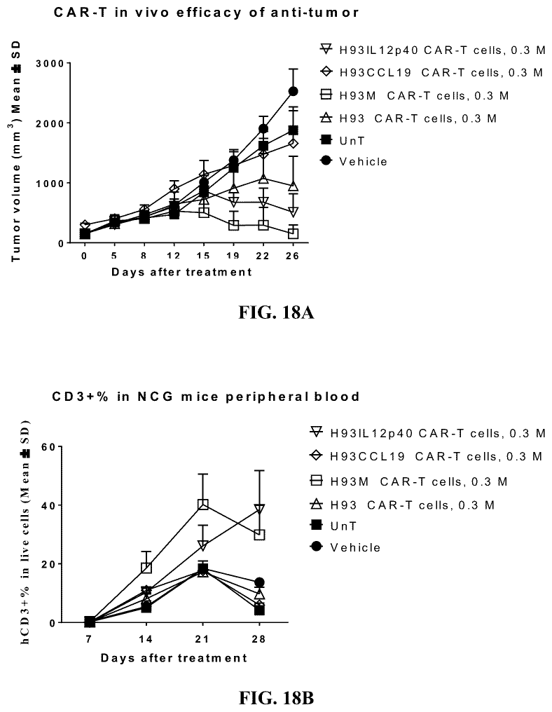

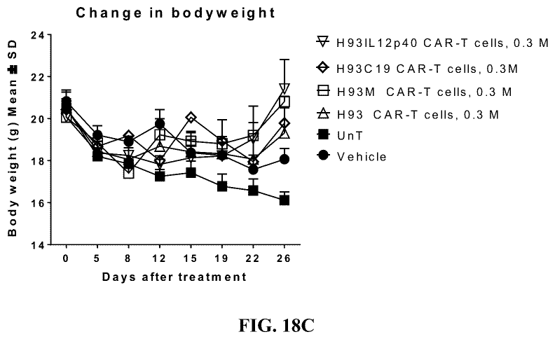

As shown in FIG. 18A, compared to the UnT group, mice administered with adoptive transfer of H93M CAR-T cells were observed to have a 92.0% reduction in tumor size (150.3 mm³ vs 1878.2 mm³) at day 26, mice administered with adoptive transfer of H93IL12p40 CAR-T cells and H93CCL19 CAR-T cells were observed to have a 72.9% and 11.7% reduction in tumor size, respectively (508.7 mm³ vs 1878.2 mm³ and 1658.7 mm³ vs 1878.2 mm³) at day 26. These results suggested H93M CAR-T cells had better therapeutic potential than H93IL 12p40 CAR-T cells and H93CCL 19 CAR-T cells.

The expansion of CAR-T cells in mouse peripheral blood cells was detected by FACS with CD3+%. H93M CAR-T cells exhibited significant expansion before day 21 and reduced subsequently. H93IL 12p40 CAR-T cells had a suboptimal proliferation ability, and H93CCL 19 CAR-T cells were similar as H93 CAR-T cells and had no obvious expansion (FIG. 18B). Concretely, the ratio of CD3 in H93M group at day 21 was 40.2%, and down to 29.8% due to reduction of tumor volume at day 28 subsequently. The ratio of CD3 in H93IL 12p40 CAR-T cells group was 26.1% at day 21 and rised to 38.5% at day 28. H93CCL 19 CAR-T cells group was similar as H93 CAR-T cells and UnT groups without obvious expansion.

No mouse bodyweight loss was observed in CAR-T groups during this experiment except the mice of UnT group had weight loss of more than 20% (FIG. 18C), and all mice of CAR-T cells treatment groups had normal appearance.

8.9.2. Comparison of p40 and CCL-19 Armored CARs with CARs Armored with Either of them Separately in C57BL/6 Syngeneic Model

Following the method disclosed in Example 1, the vector expressing mouse anti-GPC3 scFv CAR (i.e., musH93 CAR, SEQ ID NO: 27) was constructed with an anti-GPC3 scFv (SEQ ID NO: 1) and a mouse CAR backbone polypeptide (SEQ ID NO: 28) comprising from the N-terminus to the C-terminus: a mouse CD8α hinge domain, a mouse CD8a transmembrane domain, a mouse CD137 co-stimulatory signaling domain, and a mouse CD3ζ cytoplasmic signaling domain.

Based on musH93 CAR backbone, MCS in musH93M CAR vector allowed insertion of a nucleic acid sequence comprising a nucleic acid sequence encoding mouse IL 12p40 (SEQ ID NO: 29) and mouse CCL-19 (SEQ ID NO: 30) linked by a 2A self-cleaving peptide (e.g., a T2A peptide shown in SEQ ID NO: 14) fused to the C-terminus of another 2A self-cleaving peptide (e.g., a P2A fragment shown in SEQ ID NO: 13), upstream and operably linked to the C-terminus of CD3ζ cytoplasmic signaling domain. The nucleic acid sequence encoding the P2A peptide and mouse IL12p40-T2A-CCL-19 peptide was chemically synthesized and cloned into pMSCV-BBzBB CAR backbone by CloneEZ method. The resulting musH93M CAR comprises the amino acid sequence of SEQ ID NO: 31, and the nucleic acid sequence of SEQ ID NO: 32. Analogously, musH93IL 12p40 CAR (SEQ ID NO: 33) and musH93CCL19 CAR (SEQ ID NO: 34) comprising mouse IL12p40 and mouse CCL-19, respectively, were also constructed.

To better elucidate the synergy in musH93M CAR-T cells, musH93M CAR-T cells, musH93IL12p40 CAR-T cells, musH93CCL19 CAR-T cells and musH93 CAR-T cells were prepared and evaluated in C57BL/6 syngeneic model. For the subcutaneous xenograft model, C57BL/6 mice aged 6-7 weeks were inoculated with LL/2-hGPC3 cells (LL/2 cells overexpressing hGPC3) subcutaneously in the right foreleg (1.0×10⁶ cells/mouse). When the average tumor volume approached to 100 mm³ (day 9 after the xenograft inoculation), mice were randomized into 5 groups and treated with musH93 CAR-T cells (at 10 M dosage), musH93M CAR-T cells (at 6 M dosage), musH93IL 12p40 CAR-T cells (at 6 M dosage), musH93CCL19 CAR-T cells (at 6 M dosage), UnT (at 24.1 M dosage, same cell number of total T cells with CAR-T groups), respectively, by tail vein injection. Tumor size was measured with digital calipers twice per week. Tumor volume was calculated according to the following formula: Tumor volume=((length)×(width)2)/2. The expansion of CAR-T cells in peripheral blood were detected by FACS staining anti-mouse CD3 (Biolegend #100236) and CAR positive rates (Genscript #CP0001) on the day 1, 7, 14, 21 and 28 after the adoptive transfer, respectively.

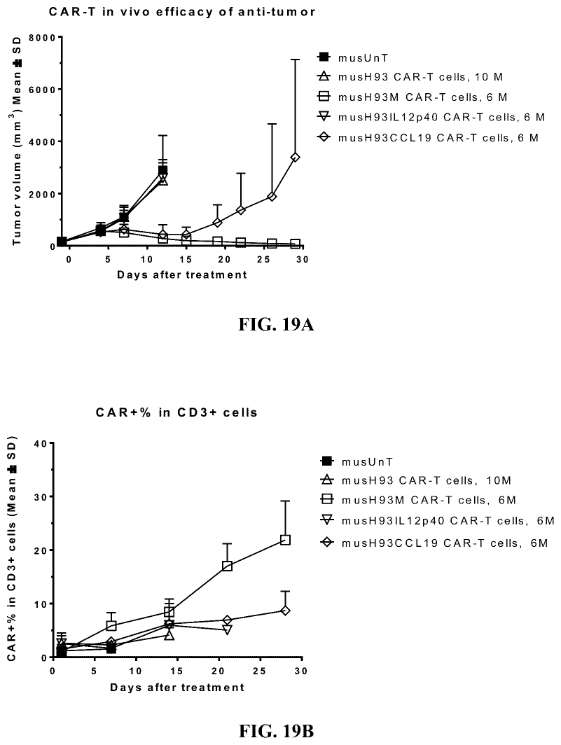

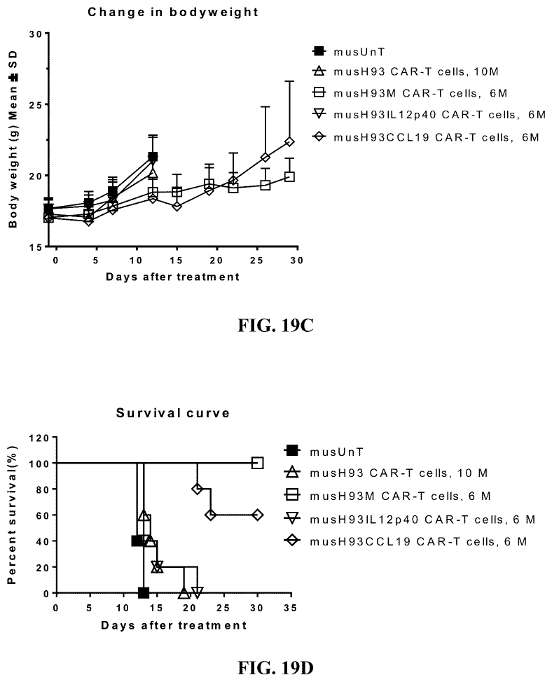

As shown in FIGS. 19A and 19D, all mice of naked musH93 CAR-T group and UnT group were sacrificed before day 19 because of large tumor volume (>3000 mm³). mice administered with adoptive transfer of musH93M CAR-T cells were almost tumor free (91.5 mm³) at day 29. Mice administered with adoptive transfer of musH93CCL19 CAR-T cells were observed to have obvious reduction in tumor size (437.2 mm³) at day 15, but tumor relapse were occurred subsequently. It is worth noting that the mice administered with adoptive transfer of musH93IL 12p40 CAR-T cells had no obvious therapeutic effect throughout the experiment. These results suggested musH93M CAR-T cells had better therapeutic potential than musH93IL 12p40 CAR-T cells and musH93CCL 19 CAR-T cells, which were consistent with that in NCG mouse xenograft model above.

The expansion of CAR-T cells in mouse peripheral blood cells was detected by FACS. MusH93M CAR-T cells exhibited significant expansion before day 28, while musH93CCL19 CAR-T cells had no obvious proliferation ability and musH93IL12p40 CAR-T cells were similar as musH93 CAR-T cells having negligible expansion (FIG. 19B). Concretely, the ratio of CD3 and CAR positive in musH93M CAR-T cells group at day 21 was 17.0%, and increased to 21.9% at day 28. MusH93CCL 19 CAR-T cells group was 6.9% at day 21 and rised to 8.7% at day 28.

No mouse bodyweight loss was observed in CAR-T groups during this experiment (FIG. 19C), and all mice of CAR-T cells treatment groups had normal appearance.

### 8.10. Example 10—Validation of the p40 and CCL-19 Armored CARs in Gastric Cancer Model

### 8.10.1. Preparation of 261M CAR-T Cells Expressing Exogenously Introduced p40 and CCL-19

Following the method disclosed in Example 1, an anti-Claudin18.2 VHH CAR (i.e., 261 CAR, SEQ ID NO: 41) disclosed in the PCT patent application NO. PCT/CN2020/139143 was constructed comprising an anti-Claudin18.2 VHH domain (SEQ ID NO: 40) and a CAR backbone polypeptide comprising from the N-terminus to the C-terminus: a CD8α hinge domain (SEQ ID NO: 8), a CD8α transmembrane domain (SEQ ID NO: 9), a CD137 co-stimulatory signaling domain (SEQ ID NO: 10), and a CD3ζ cytoplasmic signaling domain (SEQ ID NO: 11). The structure of 261M CAR (SEQ ID NO: 42), same as H93M CAR, comprising IL12p40-T2A-CCL-19, was also constructed.

261 CAR-T cells and 261M CAR-T cells (with secretion of IL12p40 and CCL-19) were prepared using the methods described in Example 1. On day 5 after transduction, the CAR positive rates of naked 261 CAR-T cells and armored 261M CAR-T cells were 50.7% and 23.0%, respectively.

### 8.10.2. IL-23 and CCL-19 Expression

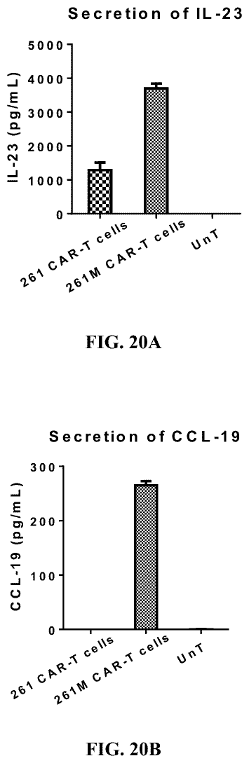

In order to verify the structure of 261M CAR, the CAR-T cells were co-cultured with Claudin18.2 positive cell line-NUGC4 for overnight and the secretion of IL-23 and CCL-19 were analyzed. Briefly, CAR-T cells and NUGC4 cell were co-cultured with F/T=1:1 in 6-well plates, and the supernatants were harvested after overnight. The human IL-23 kit (CISBIO #62HIL23PEG) and CCL-19 ELISA kit (Abcam #ab100601) were used to determine the amount of IL-23 and CCL-19 according to manufacturer's protocol. As shown in FIGS. 20A and 20B, only the 261M CAR-T cells had the high level secretion of IL-23 (3703.3 pg/mL) and CCL-19 (265.1 pg/mL) simultaneously.

### 8.10.3. In Vitro Repeat Challenge Assay for 261M CAR-T Cells Expressing Exogenously Introduced p40 and CCL-19

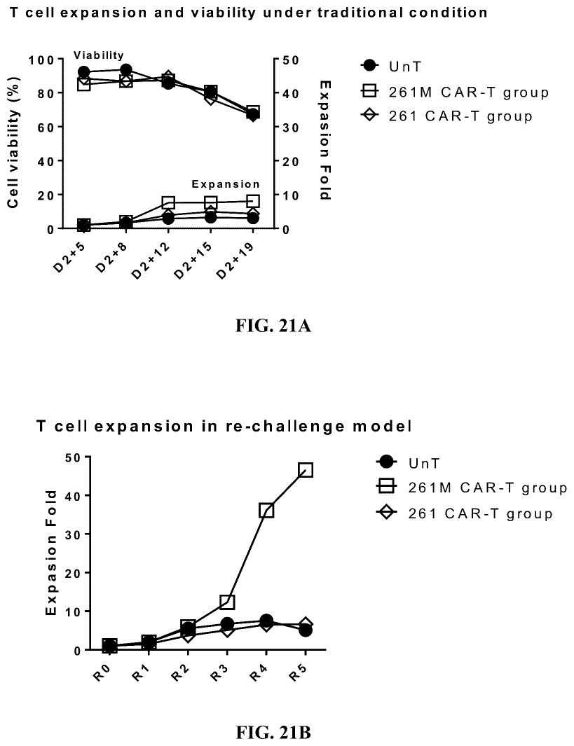

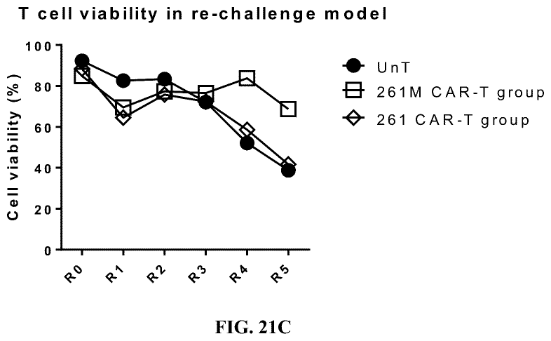

CAR-T cells repeat challenge model was set up as described in Example 2 above. CLDN18.2 positive cell line NUGC4 cells were used as the target cell. For the first round (round 1) of re-challenge, CAR-T cells were co-cultured with tumor cells at 1:1 E/T ratio in a 6-well plate. After overnight, CAR-T cells in the well were gently collected, centrifuged, and re-suspended in fresh CAR-T culture medium (RPMI-1640+300 IU/mL IL-2). Then CAR-T cells were added to a new plate for another 2 days. Where after, CAR-T cells in the well were collected, re-suspended in fresh medium again and added to a new plate seeded with fresh tumor cells (round 2). CAR-T cells were stimulated for 5 rounds in total. T cell number and viability were counted every round. There were three groups of treatment in re-challenge assay: 261 CAR-T group (the initial CAR-T cells were 262 CAR-T cells), 261M CAR-T group (the initial CAR-T cells were 261M CAR-T cells) and UnT (the initial T cells were T cells un-transduced with CAR). To determine the benefits of the expansion function of CAR-T cells with IL 12p40 expression in the re-challenge assay, the number of T cells were recorded at the end of each round and the expansion folds were calculated. As shown in FIG. 21B, repeat stimulations with NUGC4 cells resulted in a significant expansion of 261M CAR-T group (46.6 folds at round 5) in comparison with 261 CAR-T group (6.7 folds at round 5) and UnT group (5.1 folds at round 5). The cell viability of 261M CAR-T cells (68.7% at round 5) was higher than naked 261 CAR-T cells (41.7% at round 5) and UnT cells (38.8% at round 5) (FIG. 21C). Compared with the similar T cell expansion and viability of the three groups under traditional culture condition (10%1640+300 IU IL-2), 261M CAR-T cells (46.6 folds at round 5) after multiple challenged by NUGC4 cells showed antigen dependent expansion (FIGS. 21A and 21B).

8.10.4. Anti-Tumor Effect of CAR-T Cells Expressing Exogenously Introduced p40 and CCL-19 after Adoptive Transfer in Gastric Cancer Model

Therapeutic efficacy of 261M CAR-T cells expressing exogenously introduced p40 and CCL-19 after adoptive transfer were evaluated in gastric cancer model. 261M CAR-T cells and naked 261 CAR-T cells were prepared and evaluated in NUGC4 xenograft model. For the subcutaneous xenograft model, NCG mice aged 6-7 weeks were inoculated with NUGC4 cells subcutaneously in the right foreleg (3.0×10⁶ cells/mouse). When the average tumor volume approached to 100 mm³ (day 14 after the xenograft inoculation), mice were randomized into 3 groups and treated with 261 CAR-T cells (at dosage of 0.5 M), 261M CAR-T cells (at dosage of 0.5 M) and UnT cells (at dosage of 1.9 M), respectively, by tail vein injection. Tumor size was measured with digital calipers twice per week. Tumor volume was calculated according to the following formula: Tumor volume=(length)×(width)2/2.

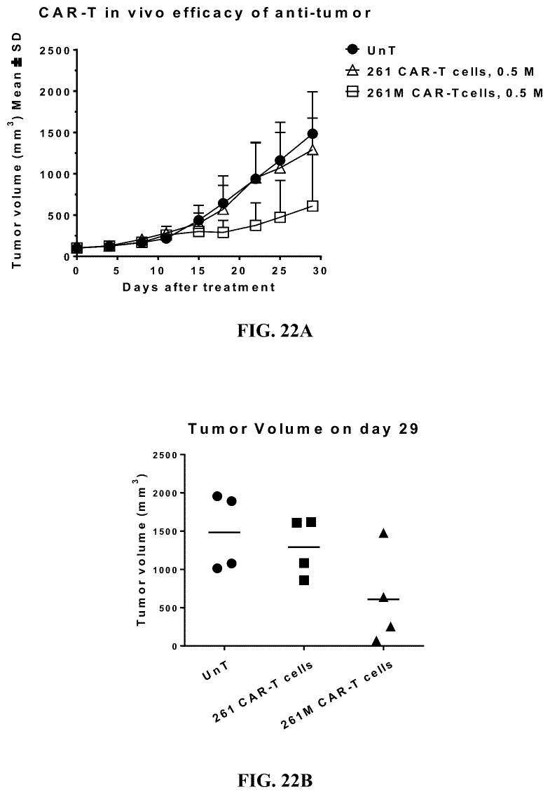

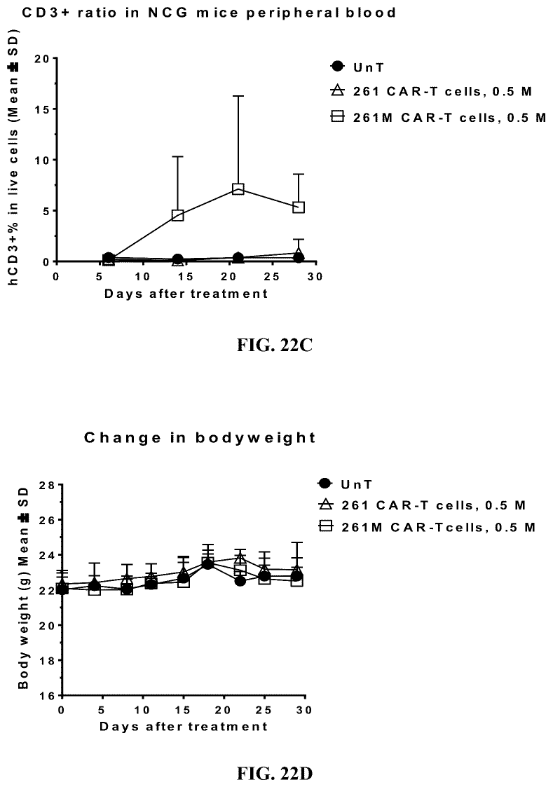

As shown in FIGS. 22A and 22B, compared to the UnT and 261 CAR-T cells groups, mice administered with adoptive transfer of 261M CAR-T cells were observed to have a 59.0% and 52.9% reduction respectively in tumor size (609.0 mm³ vs 1484.8 mm³, 1291.8 mm³) at day 29, which suggested 261M CAR-T cells had better therapeutic potential than naked 261CAR-T cells at lower dosage (0.5 M/mouse).

The expansion of CAR-T cells in mouse peripheral blood cells was detected by FACS. And 261M CAR-T cells exhibited significant expansion after day 5 at 0.5 M dosage with the ratio of CD3 positive T cells reaching highest (7.1%) on day 21 and decreasing subsequently on day 28 (5.3%), while there were no obvious expansion in the mice treated with naked mus261 CAR-T cells and UnT throughout the treatment (FIG. 22C). These data indicate that 261M CAR-T cells expressing exogenously introduced p40 and CCL-19 have excellent expansion ability in vivo and better therapeutic potential (FIGS. 22A and 22C), which are consistent with that in GPC3 mouse model.

No mouse bodyweight loss was observed in all the three groups during this experiment (FIG. 22D), and the mice of treatment groups had normal appearance.

### 8.11. Example 11—Anti-Tumor Effect of CAR-T Cells Expressing Exogenously Introduced p40 and CCL-19 and CAR-T Cells Expressing Exogenously Introduced IL-7 and CCL-19 after Adoptive Transfer In Vivo

Following the method disclosed in Example 1, a mouse anti-Claudin18.2 VHH CAR (i.e., mus261 CAR, SEQ ID NO: 35) was constructed with an anti-Claudin18.2 VHH domain (SEQ ID NO: 40) and a mouse CAR backbone polypeptide (SEQ ID NO: 31) comprising from the N-terminus to the C-terminus: a mouse CD8α hinge domain, a mouse CD8α transmembrane domain, a mouse CD137 co-stimulatory signaling domain, and a mouse CD3ζ cytoplasmic signaling domain.

Based on mus261 CAR backbone, MCS in mus261M CAR vector allowed insertion of a nucleic acid sequence comprising a nucleic acid sequence encoding mouse IL 12p40 (SEQ ID NO: 29) and mouse CCL-19 (SEQ ID NO: 30) linked by a 2A self-cleaving peptide (e.g., a T2A peptide shown in SEQ ID NO: 14) fused to the C-terminus of another 2A self-cleaving peptide (e.g., a P2A fragment shown in SEQ ID NO: 13), upstream and operably linked to the C-terminus of CD3ζ cytoplasmic signaling domain. The nucleic acid sequence encoding the P2A peptide and mouse IL12p40-T2A-mouse CCL-19 peptide was chemically synthesized and cloned into pMSCV-BBzBB CAR backbone via clone EZ method. The resulting mus261M CAR comprises the amino acid sequence of SEQ ID NO: 44, and the nucleic acid sequence of SEQ ID NO: 45. Analogously, the nucleic acid sequence encoding the F2A peptide and mouse IL7-F2A-mouse CCL 19 peptide (SEQ ID NO: 36) was chemically synthesized and cloned into pMSCV-BBzBB CAR backbone via clone EZ method to form the mus261-719 CAR.

Therapeutic efficacy of mus261M CAR-T cells expressing exogenously introduced IL12p40 and CCL-19 and mus261-719 CAR-T cells expressing exogenously introduced IL-7 and CCL-19 after adoptive transfer were evaluated in syngeneic gastric cancer model. Mus261M CAR-T cells and mus261-719 CAR-T cells were prepared and evaluated in C57BL/6 syngeneic xenograft model. For the subcutaneous xenograft model, C57BL/6 mice aged 6-7 weeks were inoculated with LL/2-hCLDN18.2 cells (LL/2 cells overexpressing human Claudin18.2) subcutaneously in the right foreleg (1.0×10⁶ cells/mouse). When the average tumor volume approached to 100 mm³ (day 9 after the xenograft inoculation), mice were randomized into 4 groups and treated with mus261 CAR-T cells (at dosage of 10 M), mus261M CAR-T cells (at dosage of 10 M), mus261-719 CAR-T cells (at dosage of 10 M) and solvent only (Vehicle), respectively, by tail vein injection. Tumor size was measured with digital calipers twice per week from the day before the mice receiving the treatment. Tumor volume was calculated according to the following formula: Tumor volume=((length)×(width)²)/2.

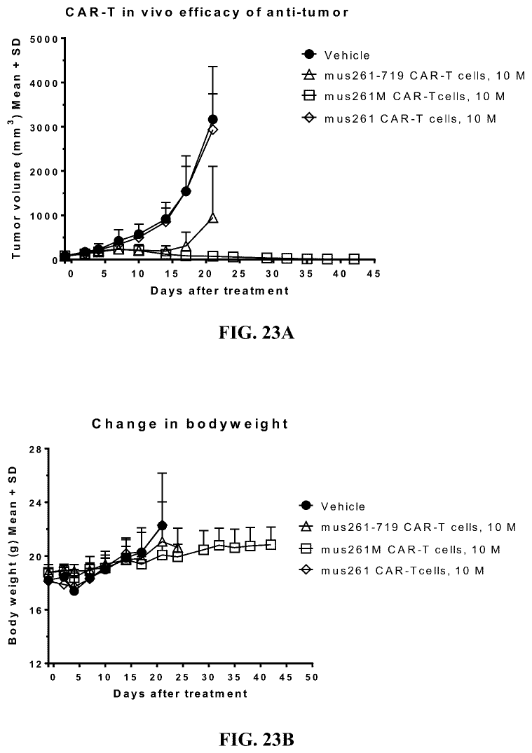

As shown in FIG. 23A, compared to the Vehicle and mus261 CAR-T cells groups, mice administered with the adoptive transfer of mus261M CAR-T cells were tumor free and had a long-lasting efficacy until day 42. Mus261-719 CAR-T cells treatment group was observed obvious inhibitory effect on tumor growth with a growth inhibition rate of 79.0% at day 14 compared with Vehicle group (191.9 mm³ vs 914.8 mm³), but the tumor sizes were increased subsequently and all mice were sacrificed because of the rupture and scab of tumors on day 21. This result suggested mus261M CAR-T cells (with secretion of musIL12p40 and musCCL-19) had better therapeutic potential than mus261-719 CAR-T cells (with secretion of musIL7 and musCCL-19).

No mouse bodyweight loss was observed in CAR-T groups during this experiment (FIG. 23B).

### 8.12. Example 12—Preparation and Characterization of CAR-T Cells Expressing Membrane-Bound IL12p40 and CCL-19

In order to further improve the function of IL12p40 and CCL-19 armored CAR-T cells, the exemplary membrane-bound form of IL 12p40 (MB12) was constructed. Based on H93 CAR backbone, MCS in H93M-MB12 CAR vector allowed insertion of a nucleic acid sequence comprising a nucleic acid sequence encoding membrane-bound IL 12p40 (i.e., MB12, SEQ ID NO: 37) and human CCL 19 (SEQ ID NO: 6). Concretely, human IL 12p40 was linked by a nucleic acid sequence encoding of CD8α Hinge, CD8α transmembrane domain, and CD8α intracellular cytoplasmic domain, so that IL 12p40 could be anchored on cell membrane. MB12 was fused to the N-terminus of 2A self-cleaving peptide (e.g., a T2A fragment shown in SEQ ID NO: 14), downstream and operably linked to human CCL-19. The nucleic acid sequence encoding MB12 and CCL 19 was chemically synthesized and cloned into pLSINK-BBzBB CAR backbone via the HpaI (5′-GTTAAC-3′) (SEQ ID NO: 17) and MluI (5′-ACGCGT-3′) (SEQ ID NO: 23) restriction sites. The resulting H93M-MB12 CAR comprises the amino acid sequence of SEQ ID NO: 38, and the nucleic acid sequence of SEQ ID NO: 39. H93M-MB12 CAR-T cells were prepared following the procedures disclosed in Example 1.

### 8.12.1. Expression of IL12p40 Protein on Membrane of CAR-T Cells

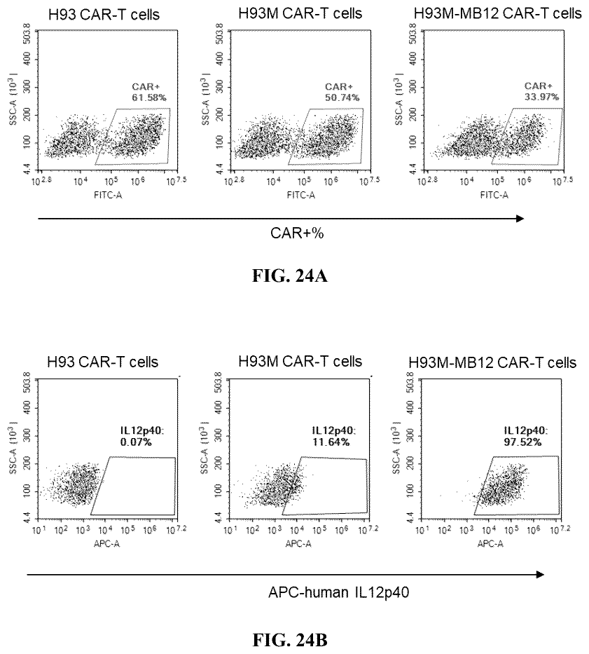

To validation of membrane bound strategy, the expression of IL 12p40 protein on membrane was detected by FACS staining anti-human IL12p40 (Biolegend #501809) and CAR positive rates (Genscript #CP0001). As shown in FIG. 24A, the CAR positive rates of naked H93 CAR-T cells, H93M CAR-T cells and H93M-MB12 CAR-T cells were 61.58%, 50.74%, and 33.97%, respectively. As shown in FIG. 24B, H93M-MB12 CAR-T cells had the expression of IL 12p40 protein on membrane closely to 100%, indicating the feasibility of membrane bound strategy initially.

### 8.12.2. Secretion of IL12p40, IL-23, CCL-19 and IL-12 Proteins

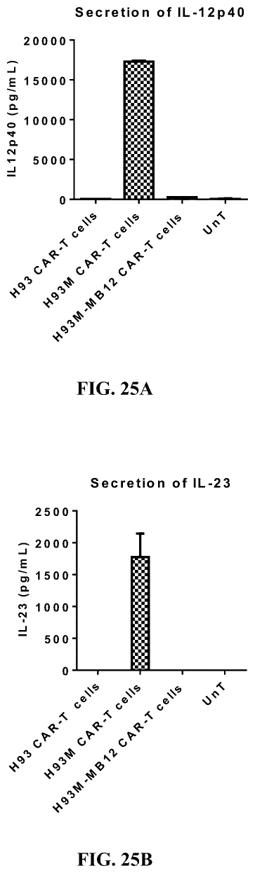

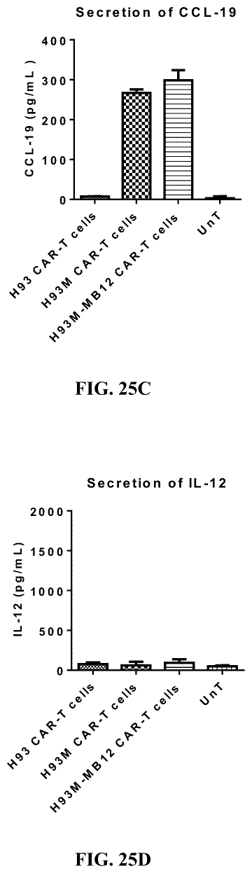

In order to further verify the feasibility of membrane bound strategy, the secretion of IL 12p40 and IL-23 were analyzed. Briefly, cells cocultured with anti-CD3/CD28 (RPMI-1640+300 IU/mL IL-2+anti-CD3/CD28) were cultured at 1×10⁶ cells/mL in 6-well plates and supernatants were harvested after overnight. The human IL12p40 kit (Invitrogen #4215608), IL-23 kit (Cisbio #62HIL23PEG) and CCL-19 kit (Abcam #ab100601) were used to determine the amount of IL 12p40, IL-23 and CCL-19 according to manufacturer's protocol. As shown in FIGS. 25A and 25B, the IL12p40-secretory H93M CAR-T cells had higher secretion of IL 12p40 (17288.6 pg/mL vs 41.1 pg/mL) and IL23 (1775.1 pg/mL vs 0 pg/mL) compared with H93 CAR-T cells after coculture with anti-CD3/CD28, while the secretion of IL 12p40 (267.7 pg/mL) in H93M-MB12 CAR-T cells was reduced significantly and no obvious secretion of IL23 after coculture with anti-CD3/CD28. Moreover, H93M CAR-T cells and H93M-MB12 CAR-T cells had the same level of CCL-19 secretion (FIG. 25C). The data indicate the membrane-bound IL 12p40 structure of MB12 was feasible. As shown in FIG. 25D, there was no obvious secretion of IL-12 in all the test cells after coculture with anti-CD3/CD28, indicating that although H93M CAR-T cells secrete IL12p40, they only produce IL-23 not IL-12.

### 8.12.3 Cell Migration Assay for IL12p40 Membrane-Bound CAR-T Cells

To evaluate the chemotactic function of CCL-19 in H93M-MB12 CAR-T cells and H93M CAR-T cells, cell migration assay was performed. Chemotaxis of the responder T cells was measured by migration through a polycarbonate filter of 5-μm pore-size in 96-well transwell chambers (Corning). CAR-T cells and UnT cells were stimulated with GPC3 positive human HCC cell line PLC/PRF/5 cells and the co-culture supernatant was collected after 24 hours. Then 125 μL of supernatant was placed in the lower chambers, and 75 μL untreated T cells (0.075 M) were incubated in the upper chambers. After 2, 4 and 6 hours, the T cells migrated from the upper chamber to the lower chamber were counted by blood counting chamber. There were four groups of treatment in cell migration assay: H93 CAR-T group (the supernatant in the lower chamber was from H93 CAR-T cells co-cultured with PLC/PRF/5 cells), H93M CAR-T group (the supernatant in the lower chamber was from H93M CAR-T cells co-cultured with PLC/PRF/5 cells), H93M-MB12 CAR-T group (the supernatant in the lower chamber was from H93M-MB12 CAR-T cells co-cultured with PLC/PRF/5 cells) and UnT group (the supernatant in the lower chamber was from UnT cells co-cultured with PLC/PRF/5 cells).

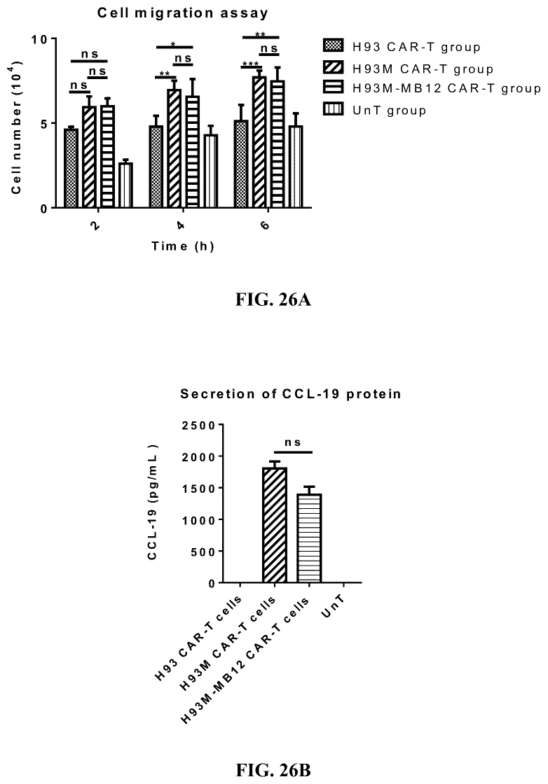

As shown in FIG. 26A, more T cells were attracted into the supernatant of H93M CAR-T cells and H93M-MB12 CAR-T cells co-cultured with PLC/PRF/5 cells than that of H93 CAR-T cells co-cultured with PLC/PRF/5 cells and that of UnT cells co-cultured with PLC/PRF/5 cells at 4 h and 6 h. Concretely, H93M CAR-T and H93M-MB12 CAR-T groups could completely attract all T cells of upper chambers on 6 hours (0.077 M and 0.075 M), while H93 CAR-T group and UnT group with no production of CCL-19 only migrated fewer T cells even on 6 hours, 0.051 M and 0.048 M respectively. In addition, the concentration of CCL-19 in co-culture supernatant was measured by CCL-19 ELISA (Abcam #ab100601). The concentration of CCL-19 in the supernatant from H93M-MB12 CAR-T cells and H93M CAR-T cells co-cultured with PLC/PRF/5 cells was 1389.1 pg/mL and 1804.7 pg/mL (p<0.005), respectively (FIG. 26B). This result indicates that H93M-MB12 CAR-T cells have the same chemotactic function as H93M CAR-T cells in vitro.

### 8.12.4. Re-Challenge Assay for IL12p40 Membrane-Bound CAR-T Cells

Repeat challenge model was set up as described in Example 2 above. GPC3 positive cell line-Hep3B cells was used as the target cell. For the first round (round 1) of re-challenge, CAR-T cells were co-cultured with tumor cells at 2:1 E/T ratio in a 6-well plate. After overnight, CAR-T cells in the well were gently collected, centrifuged, and re-suspended in fresh CAR-T culture medium (RPMI-1640+300 IU/mL IL-2). Then CAR-T cells were added to a new plate for another 2 days. Where after, CAR-T cells in the well were collected, re-suspended in fresh medium again and added to a new plate seeded with fresh tumor cells (round 2). CAR-T cells were stimulated for 5 rounds in total. T cells were counted every round and the cytokines were detected overnight after each stimulation. There were four groups of treatment in re-challenge assay: H93 CAR-T group (the initial CAR-T cells were H93 CAR-T cells), H93M CAR-T group (the initial CAR-T cells were H93M CAR-T cells), H93M-MB12 CAR-T group (the initial CAR-T cells were H93M-MB12 CAR-T cells) and UnT group (the initial T cells were T cells un-transduced with CAR).

### 8.12.4.1. Expansion of T Cells

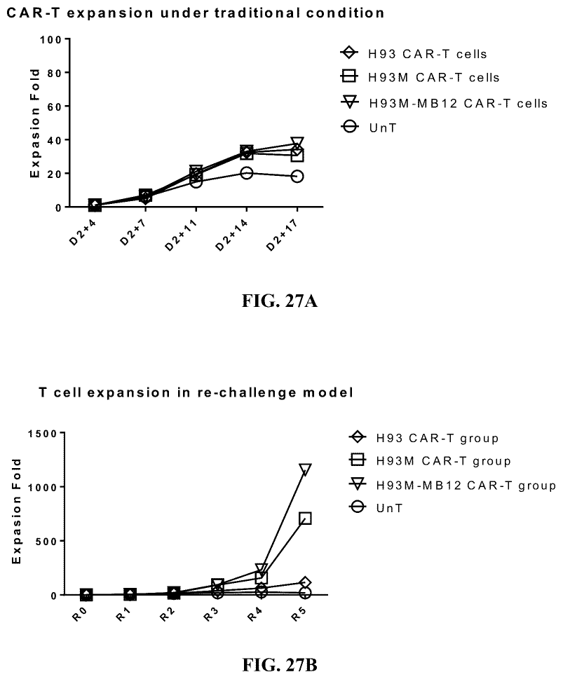

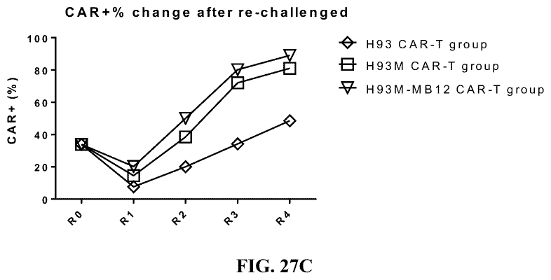

To determine the benefits of the expansion function of CAR-T cells with membrane-bound IL12p40 expression in the re-challenge assay, the number of T cells were recorded at the end of each round and the expansion folds were calculated. As shown in FIG. 27B, repeat stimulations with Hep3B cells resulted in a significant expansion in H93M-MB12 CAR-T group (1155.7 folds at round 5), in comparison with H93M CAR-T group (708.1 folds at round 5) and H93 CAR-T group (115.1 folds at round 5), which suggested that IL 12p40 membrane-bound CAR-T cells still had the benefits of the expansion function of CAR-T cells. However, the IL 12p40 membrane-bound CAR-T cells in traditional culture condition without antigen stimulation did not displayed better expansion advantage, which ascribed the antigen dependent expression of IL23 (FIG. 27A).

The CAR positive rates of H93M-MB12 CAR-T cells and H93M CAR-T cells were calculated after each round. As shown in FIG. 27C, H93M-MB12 CAR-T cells had faster increasing of CAR positive rate with the rounds of stimulation than H93M CAR-T cells (89.0% vs 81.0% after round 4), naked H93 CAR-T cells had slower increasing of CAR positive rate (48.5% after round 4).

### 8.12.4.2. Secretion of Cytokines TNF-α and IFN-γ

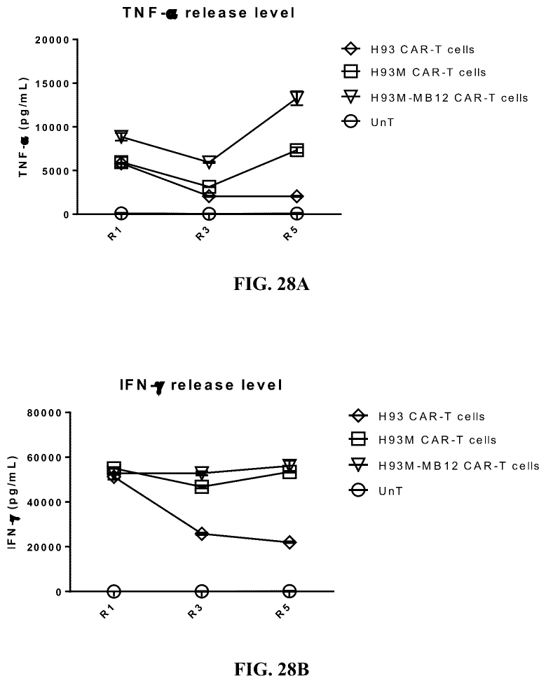

To further verify the sustained cytotoxic activity of H93M-M12 CAR-T cells against GPC3 positive tumor cells in comparison with H93 CAR-T cells in the re-challenge assay, TNF-α and IFN-γ production were analyzed by ELISA after coculturing CAR-T cells with Hep3B cells at 2:1 E/T ratio for overnight. As a result, the abilities to produce TNF-α and IFN-γ of H93M-M12 CAR-T group were significant higher compared with that of H93 CAR-T group or even that of H93M CAR-T group in all rounds. As shown in FIGS. 28A and 28B, the concentrations of TNF-α and IFN-γ secreted by cells in H93M-MB12 CAR-T group are higher than other groups after round 3 to round 5, within the range of 5939.4-13287.2 pg/mL (TNF-α) and 52842.5-56072.5 pg/mL (IFN-γ). H93M CAR-T cells also had high production level, within the range of 3134.2-7343.9 pg/mL (TNF-α) and 46799.6-53462.7 pg/mL (IFN-γ), while the concentrations of TNF-α and IFN-γ secreted by cells in H93 CAR-T group were gradually reduced after round 1 to round 5, within the range of 5823.0-2065.3 pg/mL (TNF-α) and 51265.5-21996.2 pg/mL (IFN-γ).

The teachings of all patents, published applications and references cited herein are incorporated by reference in their entirety. While example embodiments have been particularly shown and described, it will be understood by those skilled in the art that various changes in form and details may be made therein without departing from the scope of the embodiments encompassed by the appended claims.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed:

1. An immune effector cell expressing
(i) an exogenously introduced p40 subunit of IL-12;
(ii) an exogenously introduced ligand of CCR7; and
(iii) a functional exogenous receptor comprising:
(a) an extracellular antigen binding domain,
(b) a transmembrane domain, and
(c) an intracellular signaling domain.
wherein the ligand of CCR7 is CCL-19, or wherein the ligand of CCR7 is CCL-21.

2. The immune effector cell of claim 1, wherein the p40 is a human p40 or a fragment or variant thereof.

3. The immune effector cell of claim 2, wherein the p40 comprises the amino acid sequence of SEQ ID NO: 5.

4. The immune effector cell of claim 2, wherein the p40 comprises an amino acid sequence having at least 75%, 80%, 90%, 91%, 92%, 93%, 94%, 95%, 96%, 97%, 98%, or 99% identity to the amino acid sequence of SEQ ID NO: 5.

5. The immune effector cell of any one of claims 1 to 4, wherein the CCL-19 is a human CCL-19 or a fragment or variant thereof; and/or wherein CCL-21 is a human CCL-21 or a fragment or variant thereof.

6. The immune effector cell of claim 5, wherein the CCL-19 comprises the amino acid sequence of SEQ ID NO: 6, or wherein the CCL-19 comprises an amino acid sequence having at least 75%, 80%, 90%, 91%, 92%, 93%, 94%, 95%, 96%, 97%, 98%, or 99% identity to the amino acid sequence of SEQ ID NO: 6.

7. The immune effector cell of claim 5, wherein the CCL-21 comprises the amino acid sequence of SEQ ID NO: 22, or wherein the CCL-21 comprises an amino acid sequence having at least 75%, 80%, 90%, 91%, 92%, 93%, 94%, 95%, 96%, 97%, 98%, or 99% identity to the amino acid sequence of SEQ ID NO: 22.

8. The immune effector cell of any one of claims 1 to 7, wherein the functional exogenous receptor is a T cell receptor (TCR), a chimeric antigen receptor (CAR), a chimeric TCR (cTCR), or a T cell antigen coupler (TAC)-like chimeric receptor.

9. The immune effector cell of claim 8, wherein the functional exogenous receptor is a CAR.

10. The immune effector cell of any one of claims 1 to 9, wherein the transmembrane domain is derived from a molecule selected from the group consisting of CD8α, CD4, CD28, CD137, CD80, CD86, CD152 and PD1.

11. The immune effector cell of claim 10, wherein the transmembrane domain is from CD8α or CD28.

12. The immune effector cell of any one of claims 1 to 11, wherein the intracellular signaling domain comprises a primary intracellular signaling domain of an immune effector cell.

13. The immune effector cell of claim 12, wherein the primary intracellular signaling domain is from CD3ζ.

14. The immune effector cell of any one of claims 1 to 13, wherein the intracellular signaling domain comprises a co-stimulatory signaling domain.

15. The immune effector cell of claim 14, wherein the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof.

16. The immune effector cell of claim 15, wherein the co-stimulatory signaling domain comprises a cytoplasmic domain of CD28 and/or a cytoplasmic domain of CD137.

17. The immune effector cell of any one of claims 1 to 16, further comprising a hinge domain located between the C-terminus of the extracellular antigen binding domain and the N-terminus of the transmembrane domain.

18. The immune effector cell of claim 17, wherein the hinge domain is from CD8α.

19. The immune effector cell of any one of claims 1 to 18, further comprising a signal peptide located at the N-terminus of the polypeptide.

20. The immune effector cell of claim 19, wherein the signal peptide is from CD8α.

21. The immune effector cell of any one of claims 1 to 20, wherein the immune effector cell is a T cell.

22. A polypeptide comprising:
(i) an exogenously introduced p40 subunit of IL-12;
(ii) an exogenously introduced ligand of CCR7; and
(iii) a functional exogenous receptor comprising:
(a) an extracellular antigen binding domain,
(b) a transmembrane domain, and
(c) an intracellular signaling domain.
wherein the ligand of CCR7 is CCL-19, or wherein the ligand of CCR7 is CCL-21.

23. The polypeptide of claim 22, wherein the p40 is a human p40 or a fragment or variant thereof.

24. The polypeptide of claim 22, wherein the p40 comprises the amino acid sequence of SEQ ID NO: 5.

25. The polypeptide of claim 22, wherein the p40 comprises an amino acid sequence having at least 75%, 80%, 90%, 91%, 92%, 93%, 94%, 95%, 96%, 97%, 98%, or 99% identity to the amino acid sequence of SEQ ID NO: 5.

26. The polypeptide of any one of claims 22 to 25, wherein the CCL-19 is a human CCL-19 or a fragment or variant thereof; and/or wherein the CCL-21 is a human CCL-21 or a fragment or variant thereof.

27. The polypeptide of claim 26, wherein the CCL-19 comprises the amino acid sequence of SEQ ID NO: 6, or wherein the CCL-19 comprises an amino acid sequence having at least 75%, 80%, 90%, 91%, 92%, 93%, 94%, 95%, 96%, 97%, 98%, or 99% identity to the amino acid sequence of SEQ ID NO: 6.

28. The polypeptide of claim 26, wherein the CCL-21 comprises the amino acid sequence of SEQ ID NO: 22, or wherein the CCL-21 comprises an amino acid sequence having at least 75%, 80%, 90%, 91%, 92%, 93%, 94%, 95%, 96%, 97%, 98%, or 99% identity to the amino acid sequence of SEQ ID NO: 22.

29. The polypeptide of any one of claims 22 to 28, wherein the functional exogenous receptor is a T cell receptor (TCR), a chimeric antigen receptor (CAR), a chimeric TCR (cTCR), or a T cell antigen coupler (TAC)-like chimeric receptor.

30. The polypeptide of claim 29, wherein the functional exogenous receptor is a CAR.

31. The polypeptide of any one of claims 22 to 30, wherein the transmembrane domain is derived from a molecule selected from the group consisting of CD8α, CD4, CD28, CD137, CD80, CD86, CD152 and PD1.

32. The polypeptide of claim 31, wherein the transmembrane domain is from CD8α or CD28.

33. The polypeptide of any one of claims 22 to 32, wherein the intracellular signaling domain comprises a primary intracellular signaling domain of an immune effector cell.

34. The polypeptide of claim 33, wherein the primary intracellular signaling domain is from CD3ζ.

35. The polypeptide of any one of claims 22 to 34, wherein the intracellular signaling domain comprises a co-stimulatory signaling domain.

36. The polypeptide of claim 35, wherein the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof.

37. The polypeptide of claim 36, wherein the co-stimulatory signaling domain comprises a cytoplasmic domain of CD28 and/or a cytoplasmic domain of CD137.

38. The polypeptide of any one of claims 22 to 37, further comprising a hinge domain located between the C-terminus of the extracellular antigen binding domain and the N-terminus of the transmembrane domain.

39. The polypeptide of claim 38, wherein the hinge domain is from CD8α.

40. The polypeptide of any one of claims 1 to 39, further comprising a signal peptide located at the N-terminus of the polypeptide.

41. The polypeptide of claim 40, wherein the signal peptide is from CD8α.

42. The polypeptide of any one of claims 22 to 41, wherein the p40, the CCL-19, and the functional exogenous receptor are linked to each other via a peptide linker; or wherein the p40, the CCL-21, and the functional exogenous receptor are linked to each other via a peptide linker.

43. The polypeptide of claim 42, wherein the peptide linker is a 2A self-cleaving peptide optionally selected from a group consisting of F2A, E2A, P2A, T2A, and variants thereof.

44. The polypeptide of claim 43, wherein the 2A self-cleaving peptide is a P2A fragment comprising the amino acid sequence of SEQ ID NO: 13.

45. The polypeptide of claim 43, wherein the 2A self-cleaving peptide is a T2A fragment comprising the amino acid sequence of SEQ ID NO: 14.

46. The polypeptide of any one of claims 22 to 45, wherein the p40 and the CCL-19 are present in a domain comprising the amino acid sequence of SEQ ID NO: 4, or wherein the p40 and the CCL-19 are present in a domain comprising an amino acid sequence having at least 75%, 80%, 90%, 91%, 92%, 93%, 94%, 95%, 96%, 97%, 98%, or 99% identity to the amino acid sequence of SEQ ID NO: 4.

47. The polypeptide of any one of claims 22 to 45, wherein the p40 and the CCL-21 are present in a domain comprising the amino acid sequence of SEQ ID NO: 20, or wherein the p40 and the CCL-21 are present in a domain comprising an amino acid sequence having at least 75%, 80%, 90%, 91%, 92%, 93%, 94%, 95%, 96%, 97%, 98%, or 99% identity to the amino acid sequence of SEQ ID NO: 20.

48. An isolated nucleic acid comprising a nucleic acid sequence encoding the polypeptide of any one of claims 22 to 47.

49. An isolated nucleic acid comprising:
(i) a first region encoding an exogenously introduced p40 subunit of IL-12;
(ii) a second region encoding an exogenously introduced ligand of CCR7; and
(iii) a third region encoding a functional exogenous receptor comprising:
(a) an extracellular antigen binding domain,
(b) a transmembrane domain, and
(c) an intracellular signaling domain.
wherein the ligand of CCR7 is CCL-19, or wherein the ligand of CCR7 is CCL-21.

50. A vector comprising the isolated nucleic acid of claim 49.

51. A method of making an immune effector cell comprising introducing into an immune cell:
(i) the nucleic acid of claim 48 or claim 49 or the vector of claim 50; or
(ii) a composition comprising two or more nucleic acids each encoding one or two of p40 subunit of IL-12, CCL-19; and a functional exogenous receptor, or a composition comprising two or more nucleic acids each encoding one or two of p40 subunit of IL-12, CCL-21; and a functional exogenous receptor.

52. An immune effector cell produced according the method of claim 51.

53. A pharmaceutical composition, comprising the immune effector cell of any one of claims 1 to 21 and 52, the polypeptide of any one of claims 22 to 47, the nucleic acid of any one of claims 48 and 49, or the vector of claim 50, and a pharmaceutically acceptable carrier.

54. A method of treating a disease or disorder in a subject, comprising administering to the subject an effective amount of the pharmaceutical composition of claim 53.


SEQUENCE LISTING (48 sequences)

SEQ ID NO: 1 - protein, 245 aa, Artificial Sequence
  Humanized anti-GPC3 scFv amino acid
DIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSS

SEQ ID NO: 2 - protein, 491 aa, Artificial Sequence
  H93 CAR amino acid
MALPVTALLLPLALLLHAARPDIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 962 aa, Artificial Sequence
  H93M CAR amino acid
MALPVTALLLPLALLLHAARPDIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVNGSGATNFSLLKQAGDVEENPGPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGEGRGSLLTCGDVEENPGPMALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 4 - protein, 447 aa, Artificial Sequence
  IL12p40-T2A-CCL-19
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGEGRGSLLTCGDVEENPGPMALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 5 - protein, 328 aa, Artificial Sequence
  Human IL12p40
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 6 - protein, 98 aa, Artificial Sequence
  Human CCL19
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 7 - protein, 21 aa, Artificial Sequence
  CD8alpha signal peptide
MALPVTALLLPLALLLHAARP

SEQ ID NO: 8 - protein, 45 aa, Artificial Sequence
  CD8alpha hinge
TTTPAPRPPTPAPTIASQPLSLRPEACRPAAGGAVHTRGLDFACD

SEQ ID NO: 9 - protein, 24 aa, Artificial Sequence
  CD8alpha transmembrane domain
IYIWAPLAGTCGVLLLSLVITLYC

SEQ ID NO: 10 - protein, 42 aa, Artificial Sequence
  4-1BB (CD137) cytoplasmic domain
KRGRKKLLYIFKQPFMRPVQTTQEEDGCSCRFPEEEEGGCEL

SEQ ID NO: 11 - protein, 112 aa, Artificial Sequence
  CD3zeta (CD3z) cytoplasmic domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - DNA, 2886 bp, Artificial Sequence
  H93M CAR nucleic acid
atggccctgcctgtgaccgctctgctgctgcctctggctctgctgctgcacgctgcccgccctgacattgtgatgacccagtctcccctgtccctgccagtgacccctggagagccagcctccatctcttgcaggagctcccagaccatcgtgcacagcaacggcaatacatacctggagtggtatctgcagaagcccggccagtcccctcagctgctgatctacaaggtgtctaacaggttcagcggagtgccagaccgctttagcggatccggatctggcaccgatttcacactgaagatctctagagtggaggccgaggacgtgggcgtgtactattgcttccaggtgagccacgtgccatatacctttggcggcggcacaaagctggagatcaagggcagcacctccggatctggcaagccaggatccggagagggatctacaaagggacaggtgcagctggtgcagagcggagcagaggtgaagaagcctggagccagcgtgaaggtgtcctgtaaggcctctggctacaccttcacagactatgagatgcactgggtgcggcaggcaaccggacagggcctggagtggatgggaggaatcgacccagagacaggcaacacagcctactcccagaagtttaagggccgggtgaccatgacaagaaataccagcatctccacagcctatatggagctgtctagcctgagatctgaggatacagccgtgtactattgtgctcgctactattcctttgcctactggggacaggggacactggtcaccgtctcatcaactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgttaacggcagcggagctacaaacttcagtcttctaaaacaggctggtgacgtggaggagaatcccggccctatgtgccatcagcagctggtcatttcttggttctctctggtgtttctggctagtcctctggtcgctatttgggaactgaaaaaagatgtgtacgtggtggagctggactggtatccagatgcaccaggagagatggtggtgctgacctgcgacacacccgaggaggatggcatcacctggacactggaccagagctccgaggtgctgggaagcggcaagaccctgacaatccaggtgaaggagttcggcgatgccggccagtacacatgtcacaagggcggcgaggtgctgtctcacagcctgctgctgctgcacaagaaggaggacggcatctggtccacagacatcctgaaggatcagaaggagcctaagaacaagaccttcctgcggtgcgaggccaagaattattctggccggttcacctgttggtggctgaccacaatctctaccgacctgaccttcagcgtgaagtctagccggggctcctctgatccacagggagtgacatgcggagcagccaccctgagcgccgagcgggtgagaggcgacaacaaggagtacgagtatagcgtggagtgccaggaggattccgcctgtccagcagcagaggagtccctgcctatcgaagtgatggtggacgccgtgcacaagctgaagtacgagaattatacaagctccttctttatcagggacatcatcaagccagatccccctaagaacctgcagctgaagcccctgaagaattcccgccaggtggaggtgtcttgggagtaccctgatacctggtccacaccacacagctatttctccctgaccttttgcgtgcaggtgcagggcaagtctaagagggagaagaaggaccgcgtgttcaccgataagacaagcgccaccgtgatctgtcggaagaacgcctccatctctgtgcgggctcaggaccgatattactcatcatcatggagtgaatgggcctcagtgccttgctcaggcagtggagagggcagaggaagtctgctaacatgcggtgacgtcgaggagaatcctggcccaatggctctgctgctggcactgagtctgctggtcctgtggacatcacctgcacctaccctgagtggaactaatgacgctgaagattgttgcctgagcgtgacccagaagccaatccccggctacatcgtgaggaacttccactatctgctgatcaaggacggatgcagggtgcctgcagtggtgtttaccacactgcggggcagacagctgtgcgcaccacctgatcagccctgggtcgagagaattattcagagactgcagcgcacctcagctaagatgaaacgccgctcaagt

SEQ ID NO: 13 - protein, 22 aa, Artificial Sequence
  P2A
GSGATNFSLLKQAGDVEENPGP

SEQ ID NO: 14 - protein, 21 aa, Artificial Sequence
  T2A
GSGEGRGSLLTCGDVEENPGP

SEQ ID NO: 15 - DNA, 6 bp, Artificial Sequence
  EcoRI Restriction Site
gaattc

SEQ ID NO: 16 - DNA, 6 bp, Artificial Sequence
  SpeI Restriction Site
actagt

SEQ ID NO: 17 - DNA, 6 bp, Artificial Sequence
  HpaI Restriction Site
gttaac

SEQ ID NO: 18 - protein, 998 aa, Artificial Sequence
  H93P CAR amino acid
MALPVTALLLPLALLLHAARPDIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVNGSGATNFSLLKQAGDVEENPGPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGEGRGSLLTCGDVEENPGPMAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 19 - DNA, 2994 bp, Artificial Sequence
  H93P CAR nucleic acid
atggccctgcctgtgaccgctctgctgctgcctctggctctgctgctgcacgctgcccgccctgacattgtgatgacccagtctcccctgtccctgccagtgacccctggagagccagcctccatctcttgcaggagctcccagaccatcgtgcacagcaacggcaatacatacctggagtggtatctgcagaagcccggccagtcccctcagctgctgatctacaaggtgtctaacaggttcagcggagtgccagaccgctttagcggatccggatctggcaccgatttcacactgaagatctctagagtggaggccgaggacgtgggcgtgtactattgcttccaggtgagccacgtgccatatacctttggcggcggcacaaagctggagatcaagggcagcacctccggatctggcaagccaggatccggagagggatctacaaagggacaggtgcagctggtgcagagcggagcagaggtgaagaagcctggagccagcgtgaaggtgtcctgtaaggcctctggctacaccttcacagactatgagatgcactgggtgcggcaggcaaccggacagggcctggagtggatgggaggaatcgacccagagacaggcaacacagcctactcccagaagtttaagggccgggtgaccatgacaagaaataccagcatctccacagcctatatggagctgtctagcctgagatctgaggatacagccgtgtactattgtgctcgctactattcctttgcctactggggacaggggacactggtcaccgtctcatcaactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgttaacggcagcggagctacaaacttcagtcttctaaaacaggctggtgacgtggaggagaatcccggccctatgtgccatcagcagctggtcatttcttggttctctctggtgtttctggctagtcctctggtcgctatttgggaactgaaaaaagatgtgtacgtggtggagctggactggtatccagatgcaccaggagagatggtggtgctgacctgcgacacacccgaggaggatggcatcacctggacactggaccagagctccgaggtgctgggaagcggcaagaccctgacaatccaggtgaaggagttcggcgatgccggccagtacacatgtcacaagggcggcgaggtgctgtctcacagcctgctgctgctgcacaagaaggaggacggcatctggtccacagacatcctgaaggatcagaaggagcctaagaacaagaccttcctgcggtgcgaggccaagaattattctggccggttcacctgttggtggctgaccacaatctctaccgacctgaccttcagcgtgaagtctagccggggctcctctgatccacagggagtgacatgcggagcagccaccctgagcgccgagcgggtgagaggcgacaacaaggagtacgagtatagcgtggagtgccaggaggattccgcctgtccagcagcagaggagtccctgcctatcgaagtgatggtggacgccgtgcacaagctgaagtacgagaattatacaagctccttctttatcagggacatcatcaagccagatccccctaagaacctgcagctgaagcccctgaagaattcccgccaggtggaggtgtcttgggagtaccctgatacctggtccacaccacacagctatttctccctgaccttttgcgtgcaggtgcagggcaagtctaagagggagaagaaggaccgcgtgttcaccgataagacaagcgccaccgtgatctgtcggaagaacgcctccatctctgtgcgggctcaggaccgatattactcatcatcatggagtgaatgggcctcagtgccttgctcaggcagtggagagggcagaggaagtctgctaacatgcggtgacgtcgaggagaatcctggcccaatggcccagagccttgctctctctctgctcattctggtgctggcattcggcatcccacgtacccagggctccgacggcggcgcgcaggactgttgcctgaagtactcccagcgcaagatccccgccaaggtggtccgcagctacaggaagcaggagccaagtctagggtgctctatccctgccatcctgtttctgccccgcaagcgcagccaggccgagctgtgcgccgacccgaaggagctttgggtgcagcagctgatgcaacacttggataaaactccgagccctcagaagcctgctcaggggtgccgaaaggaccgcggagcttccaagaccggcaaaaagggtaagggctccaaaggttgtaaacgtaccgaacggtcacagacgcccaagggaccc

SEQ ID NO: 20 - protein, 483 aa, Artificial Sequence
  IL12p40-T2A-CCL21 amino acid
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGEGRGSLLTCGDVEENPGPMAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 21 - DNA, 1449 bp, Artificial Sequence
  IL12p40-T2A-CCL21 nucleic acid
atgtgccatcagcagctggtcatttcttggttctctctggtgtttctggctagtcctctggtcgctatttgggaactgaaaaaagatgtgtacgtggtggagctggactggtatccagatgcaccaggagagatggtggtgctgacctgcgacacacccgaggaggatggcatcacctggacactggaccagagctccgaggtgctgggaagcggcaagaccctgacaatccaggtgaaggagttcggcgatgccggccagtacacatgtcacaagggcggcgaggtgctgtctcacagcctgctgctgctgcacaagaaggaggacggcatctggtccacagacatcctgaaggatcagaaggagcctaagaacaagaccttcctgcggtgcgaggccaagaattattctggccggttcacctgttggtggctgaccacaatctctaccgacctgaccttcagcgtgaagtctagccggggctcctctgatccacagggagtgacatgcggagcagccaccctgagcgccgagcgggtgagaggcgacaacaaggagtacgagtatagcgtggagtgccaggaggattccgcctgtccagcagcagaggagtccctgcctatcgaagtgatggtggacgccgtgcacaagctgaagtacgagaattatacaagctccttctttatcagggacatcatcaagccagatccccctaagaacctgcagctgaagcccctgaagaattcccgccaggtggaggtgtcttgggagtaccctgatacctggtccacaccacacagctatttctccctgaccttttgcgtgcaggtgcagggcaagtctaagagggagaagaaggaccgcgtgttcaccgataagacaagcgccaccgtgatctgtcggaagaacgcctccatctctgtgcgggctcaggaccgatattactcatcatcatggagtgaatgggcctcagtgccttgctcaggcagtggagagggcagaggaagtctgctaacatgcggtgacgtcgaggagaatcctggcccaatggcccagagccttgctctctctctgctcattctggtgctggcattcggcatcccacgtacccagggctccgacggcggcgcgcaggactgttgcctgaagtactcccagcgcaagatccccgccaaggtggtccgcagctacaggaagcaggagccaagtctagggtgctctatccctgccatcctgtttctgccccgcaagcgcagccaggccgagctgtgcgccgacccgaaggagctttgggtgcagcagctgatgcaacacttggataaaactccgagccctcagaagcctgctcaggggtgccgaaaggaccgcggagcttccaagaccggcaaaaagggtaagggctccaaaggttgtaaacgtaccgaacggtcacagacgcccaagggaccc

SEQ ID NO: 22 - protein, 134 aa, Artificial Sequence
  Human CCL21
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 23 - DNA, 6 bp, Artificial Sequence
  MluI Restriction Site
acgcgt

SEQ ID NO: 24 - protein, 177 aa, Artificial Sequence
  human IL-7?
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 25 - protein, 527 aa, Artificial Sequence
  IL12p40-P2A-IL-7 amino acid
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGATNFSLLKQAGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 26 - DNA, 1581 bp, Artificial Sequence
  IL12p40-P2A-IL7 nucleic acid
atgtgccaccagcagctggtgatcagttggttctctctggtgttcctggcctcgccacttgttgccatatgggaactcaaaaaggacgtgtacgtggtggagctggactggtaccccgacgctcccggggagatggtggtcctgacctgtgatactccggaggaagatggcatcacctggacattggatcagagctccgaggtactgggctccggcaaaactctgaccatccaggtgaaggagttcggtgatgctggacaatacacctgccataagggcggcgaggttcttagccattctctgctgctcctccacaagaaggaagacggtatttggtcgaccgacatcctgaaggaccagaaggagcccaaaaacaagacttttttgcgctgcgaggccaaaaactactccggtcgcttcacatgctggtggctaacgaccatctcaaccgacctgaccttctctgtcaagagctctcgtggctccagcgatcctcagggcgtcacctgcggggcagccaccctgagcgcagagcgcgtgagaggggacaacaaggagtatgagtactcggtggagtgtcaggaggactcggcctgcccagcggccgaggagagcctgcccattgaggtgatggtagacgccgtccacaagctgaagtacgagaactacacctcctccttctttatccgggacatcatcaagcccgacccgcccaagaacctgcagctcaagcctttgaagaattcccgccaggttgaagtcagttgggagtaccctgacacctggtctactccgcacagctatttctcgctgaccttctgcgtgcaggtccagggaaagtccaagcgcgagaagaaagacagggtgttcaccgacaagacctccgccaccgtgatttgccgaaaaaacgcctccatctccgtgcgtgcgcaggaccgctactacagttcctcttggtccgagtgggcttccgtgccttgttcaggcagcggagctacaaacttcagtcttctaaaacaggctggtgacgtggaggagaatcccggccctatgtttcatgtctccttccgctacatcttcgggctccctcccctgatcctcgttctgctgccagtggcatctagcgattgtgacattgagggcaaagacggcaagcaatacgagagcgtgctgatggtgtcgattgaccagctgttggattctatgaaagagatcggctccaactgcctgaacaacgagttcaacttcttcaagcgccacatctgtgacgccaacaaggaaggtatgtttctgttccgggccgcgcgcaagctgaggcagttcctgaaaatgaattccaccggtgatttcgacttgcacctgcttaaggtgtccgagggcacgaccatccttcttaactgcactggacaggtcaagggccgtaagcccgccgctctaggggaggctcagccgacaaagtcgctggaggagaacaagtcactcaaagaacagaagaagctcaacgacctgtgctttttgaagcgcctgctgcaggagatcaagacctgttggaataagatcctgatgggcaccaaggagcac

SEQ ID NO: 27 - protein, 504 aa, Artificial Sequence
  MusH93 CAR amino acid
MASPLTRFLSLNLLLLGESIILGSGEAKPQADIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTTTKPVLRTPSPVHPTGTSQPQRPEDCRPRGSVKGTGLDFACDIYIWAPLAGICVALLLSLIITLICSVLKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 28 - protein, 228 aa, Artificial Sequence
  mouse CAR backbone (Mouse CD8alpha Hinge-mouse CD8alpha transmembrane domain-mouse 4-1BB (CD137) cytoplasmic domain-mouse CD3zeta cytoplasmic)
TTTKPVLRTPSPVHPTGTSQPQRPEDCRPRGSVKGTGLDFACDIYIWAPLAGICVALLLSLIITLICSVLKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 29 - protein, 335 aa, Artificial Sequence
  Mouse IL12p40
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 30 - protein, 108 aa, Artificial Sequence
  Mouse CCL-19
MAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPVS

SEQ ID NO: 31 - protein, 994 aa, Artificial Sequence
  Mouse H93M CAR amino acid
MASPLTRFLSLNLLLLGESIILGSGEAKPQADIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTSTTTKPVLRTPSPVHPTGTSQPQRPEDCRPRGSVKGTGLDFACDIYIWAPLAGICVALLLSLIITLICSVLKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRVNGSGATNFSLLKQAGDVEENPGPMCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSGEGRGSLLTCGDVEENPGPMAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPVS

SEQ ID NO: 32 - DNA, 2982 bp, Artificial Sequence
  Mouse H93M CAR nucleic acid
atggcaagtcctctgactcggtttctgtctctgaatctgctcctcctcggtgaatccattatcctcggttccggtgaagctaagcctcaggccgacatcgtgatgacacagagcccactgtccctgccagtgaccccaggagagcctgctagcatctcctgtaggagctcccagacaatcgtgcacagcaacggcaacacctacctggagtggtacctgcagaagcctggacagtccccacagctgctgatctacaaggtgtctaacaggtttagcggcgtgcccgacagattctctggcagcggatccggcacagacttcaccctgaagatcagcagagtggaggccgaggatgtgggagtgtactactgttttcaggtgagccacgtgccatacacattcggcggaggcaccaagctggagatcaagggctctacaagcggatccggcaagccaggatccggagagggatctaccaagggacaggtgcagctggtgcagtccggagctgaggtgaagaagccaggagctagcgtgaaggtgtcctgcaaggcctctggctacaccttcaccgactacgagatgcactgggtgcggcaggctacaggacagggactggagtggatgggaggcatcgatcctgagacaggcaacaccgcctacagccagaagttcaagggaagggtgacaatgaccagaaacacatctatcagcaccgcttacatggagctgtctagcctgcggtccgaggacaccgccgtgtactactgcgctcgctactactcttttgcctactggggccagggaacactggtgaccgtgtcctctacatccaccacaaccaagccagtgctgaggaccccaagccctgtgcacccaacaggcacctcccagccacagaggcccgaggattgtaggcccagaggatctgtgaagggcacaggactggacttcgcctgtgatatctacatctgggctcctctggctggaatctgcgtggctctgctgctgtctctgatcatcaccctgatctgcagcgtgctgaagtggatccggaagaagttcccccacatctttaagcagcctttcaagaagacaaccggagctgctcaggaggaggacgcctgctcctgtaggtgccctcaggaagaagagggaggaggaggaggatacgagctgagggctaagttctcccgctctgctgagacagccgctaacctgcaggatccaaaccagctgtacaacgagctgaacctgggcaggagagaggagtacgacgtgctggagaagaagcgggctcgcgatcctgagatgggaggaaagcagcagcggcgcaggaacccacaggagggcgtgtacaacgctctgcagaaggacaagatggccgaggcttacagcgagatcggcaccaagggagagagacggcgcggcaagggacacgatggcctgtaccagggactctcaaccgctactaaggacacctacgatgctctccatatgcagaccctcgcaccccgcgttaacggcagcggagctacaaacttcagtcttctaaaacaggctggtgacgtggaggagaatcccggccctatgtgccctcagaaactgactatatcatggtttgccattgtgctgctggtaagtcctctcatggccatgtgggaattggagaaagatgtgtacgtggtggaggtggactggacccctgacgcaccaggagaaacagtgaacctgacgtgtgatacgccagaagaggatgacattacttggacatcagatcaaagacacggtgtcatagggtcaggcaaaactcttaccatcacagtcaaggagttcctagatgctggccagtatacctgccataaaggaggggaaaccctgtcccactcgcatctgctcctccacaagaaggaaaacggcatctggagcacagagatcctgaagaattttaagaataagaccttcttgaaatgtgaggcccccaactacagcgggaggttcacctgttcctggctggtgcaacggaacatggacctgaagttcaatattaaatcttcatcttcctccccggacagtcgcgcagttacttgtggcatggcttctttatcggcagagaaagtcacccttgaccagcgcgactatgagaagtattctgtttcctgccaggaggacgtgacgtgccccacagctgaagagactttgcctattgagttagccctggaggccagacaacagaacaaatatgaaaattattccacttcattcttcatccgtgatatcatcaagcccgatccacccaagaatcttcagatgaagcctctaaagaacagccaggtggaggttagctgggagtacccagatagctggtctaccccacattcctactttagtttaaagttttttgtaagaatccagaggaaaaaagaaaagatgaaagagactgaagaaggttgcaaccagaagggagcgtttctcgttgagaagacaagcacagaagtccagtgcaaaggtggaaatgtatgtgtgcaagctcaggacaggtactacaacagtagctgttctaagtgggcatgtgtcccctgccgagtgcggagtggcagtggagagggcagaggaagtctgctaacatgcggtgacgtcgaggagaatcctggcccaatggctcctagagtgacacctttgctggccttttctcttctagtgttgtggacgtttcctgcacccaccctgggtggagcgaatgatgcagaggactgctgcctcagtgtcacccaaaggcccataccagggaacattgtgaaggccttccgctatcttctcaatgaagatggatgtcgggttcctgctgtggtcttcactactttacgcggctaccagctgtgtgctcctccagaccagccctgggtggaccgtatcatccgaaggctgaagaaaagcagcgccaagaacaaaggcaactcaacaagaagatccccagtttcc

SEQ ID NO: 33 - protein, 865 aa, Artificial Sequence
  Mouse H93IL12p40 CAR amino acid
MASPLTRFLSLNLLLLGESIILGSGEAKPQADIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTSTTTKPVLRTPSPVHPTGTSQPQRPEDCRPRGSVKGTGLDFACDIYIWAPLAGICVALLLSLIITLICSVLKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRVNGSGATNFSLLKQAGDVEENPGPMCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 34 - protein, 638 aa, Artificial Sequence
  Mouse H93CCL19 CAR amino acid
MASPLTRFLSLNLLLLGESIILGSGEAKPQADIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTSTTTKPVLRTPSPVHPTGTSQPQRPEDCRPRGSVKGTGLDFACDIYIWAPLAGICVALLLSLIITLICSVLKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRVNGSGATNFSLLKQAGDVEENPGPMAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPVS

SEQ ID NO: 35 - protein, 379 aa, Artificial Sequence
  mus261 CAR amino acid
MASPLTRFLSLNLLLLGESIILGSGEAKPQAQVQLEESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYNYWGQGTQVTVSSTSTTTKPVLRTPSPVHPTGTSQPQRPEDCRPRGSVKGTGLDFACDIYIWAPLAGICVALLLSLIITLICSVLKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 36 - protein, 287 aa, Artificial Sequence
  mouseIL7-F2A-mouseCCL19 amino acid
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSIGSGVKQTLNFDLLKLAGDVESNPGPMAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPVS

SEQ ID NO: 37 - protein, 385 aa, Artificial Sequence
  MB12 amino acid (IL12p40-CD8alpha Hinge-CD8alpha transmembrane domain-CD8alpha intracellular cytoplasmic domain)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSTTTPAPRPPTPAPTIASQPLSLRPEACRPAAGGAVHTRGLDFACDIYIWAPLAGTCGVLLLSLVITLYCLYCNHRNRRR

SEQ ID NO: 38 - protein, 1037 aa, Artificial Sequence
  H93M-MB12 CAR amino acid
MALPVTALLLPLALLLHAARPDIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVNGSGATNFSLLKQAGDVEENPGPMDWTWILFLVAAATRVHSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSTTTPAPRPPTPAPTIASQPLSLRPEACRPAAGGAVHTRGLDFACDIYIWAPLAGTCGVLLLSLVITLYCLYCNHRNRRRGSGEGRGSLLTCGDVEENPGPMALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 39 - DNA, 3111 bp, Artificial Sequence
  H93M-MB12 CAR nucleic acid
atggccctgcctgtgaccgctctgctgctgcctctggctctgctgctgcacgctgcccgccctgacattgtgatgacccagtctcccctgtccctgccagtgacccctggagagccagcctccatctcttgcaggagctcccagaccatcgtgcacagcaacggcaatacatacctggagtggtatctgcagaagcccggccagtcccctcagctgctgatctacaaggtgtctaacaggttcagcggagtgccagaccgctttagcggatccggatctggcaccgatttcacactgaagatctctagagtggaggccgaggacgtgggcgtgtactattgcttccaggtgagccacgtgccatatacctttggcggcggcacaaagctggagatcaagggcagcacctccggatctggcaagccaggatccggagagggatctacaaagggacaggtgcagctggtgcagagcggagcagaggtgaagaagcctggagccagcgtgaaggtgtcctgtaaggcctctggctacaccttcacagactatgagatgcactgggtgcggcaggcaaccggacagggcctggagtggatgggaggaatcgacccagagacaggcaacacagcctactcccagaagtttaagggccgggtgaccatgacaagaaataccagcatctccacagcctatatggagctgtctagcctgagatctgaggatacagccgtgtactattgtgctcgctactattcctttgcctactggggacaggggacactggtcaccgtctcatcaactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgttaacggttccggcgccaccaacttttcactgttaaagcaggccggtgatgtggaggagaatccaggccctatggattggacctggatcctcttcctggtggctgcagccacccgtgtccactccatctgggagctgaagaaggacgtgtacgtggtggagctcgactggtaccccgacgctcccggggagatggtggttctgacctgtgacaccccagaggaagacggcatcacctggaccctggaccagagctccgaggtgctgggctccggcaaaactttgactatccaggtcaaggagttcggtgatgcaggccaatacacatgccataagggcggcgaggtgctgtctcattctctgctgctgctgcacaagaaggaagatggcatttggtcgactgacatcctgaaggaccagaaggagcccaagaacaagaccttcctaaggtgtgaggccaagaactattccggtcgcttcacatgttggtggctaaccacaatttcgaccgacctgacctttagcgtaaaatcctctcgtggctcgagcgatcctcagggagtcacctgcggggccgccactctgtccgccgagcgcgtgcgcggggacaacaaggagtatgagtactccgtggagtgtcaggaggacagcgcctgcccagcagccgaggagagcctccccatcgaagtgatggtggacgcggtacacaagctgaagtacgagaactacaccagctccttcttcatccgcgacatcatcaagccggacccgcccaaaaacctgcagctgaaacctttgaaaaactcccgccaggtcgaagtcagttgggagtacccggacacctggtcgactccccacagctatttcagtcttacgttctgcgtgcaggtgcagggcaagtccaaacgcgagaagaaagacagggtgttcacggacaagacttccgctaccgtcatctgccgcaagaacgcgagcatctctgtcagagctcaggaccgctactactcttccagttggtctgagtgggcgtccgtcccctgttctaccacgaccccggcgccgcgccccccaacccctgcccctaccattgcctcacagccgttgtctctgcggcccgaggcgtgccgacctgccgcgggaggcgcggtgcacacgcggggtctcgactttgcttgtgatatttacatatgggctcccctggccggcacctgcggggttttgctcctttccttggtgatcactctttactgcctgtactgcaatcaccggaaccgccgccgtggcagtggagagggcagaggaagtctgctaacatgcggtgacgtcgaggagaatcctggcccaatggctctgctgctggcactgagtctgctggtcctgtggacatcacctgcacctaccctgagtggaactaatgacgctgaagattgttgcctgagcgtgacccagaagccaatccccggctacatcgtgaggaacttccactatctgctgatcaaggacggatgcagggtgcctgcagtggtgtttaccacactgcggggcagacagctgtgcgcaccacctgatcagccctgggtcgagagaattattcagagactgcagcgcacctcagctaagatgaaacgccgctcaagt

SEQ ID NO: 40 - protein, 118 aa, Artificial Sequence
  anti-Claudin18.2 VHH domain
QVQLEESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYNYWGQGTQVTVSS

SEQ ID NO: 41 - protein, 374 aa, Artificial Sequence
  261 CAR amino acid
MASPLTRFLSLNLLLLGESIILGSGEAKPQAQVQLEESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYNYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 845 aa, Artificial Sequence
  261M CAR amino acid
MASPLTRFLSLNLLLLGESIILGSGEAKPQAQVQLEESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYNYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVNGSGATNFSLLKQAGDVEENPGPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGEGRGSLLTCGDVEENPGPMALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 43 - DNA, 2538 bp, Artificial Sequence
  261M CAR nucleic acid
atggcctcaccgttgacccgctttctgtcgctgaacctgctgctgctgggtgagtcgattatcctggggagtggagaagctaagccacaggcacaggtgcagctggaggagtctggaggaggaagcgtgcaagtgggaggctccctgaggctgtcttgcgcagcaagcggatacagatccagcgtgtgcatgggatggtttagacaggcaccaggaaaggagagggagagagtggcagtgatcggaagggacggatccaccacatatatcgattctgtgaagggccggtttaccatctccagagactctgccaagaacacactgtctctgcagatggacaatctgaagcctgaagacacagccatgtacagctgcgccgccggcctgggatactgggcttgcgaatacaactactgggggcagggcactcaggtgaccgtctcttctactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgttaacggcagcggagctacaaacttcagtcttctaaaacaggctggtgacgtggaggagaatcccggccctatgtgccaccagcagctggtgatcagttggttctctctggtgttcctggcctcgccacttgttgccatatgggaactcaaaaaggacgtgtacgtggtggagctggactggtaccccgacgctcccggggagatggtggtcctgacctgtgatactccggaggaagatggcatcacctggacattggatcagagctccgaggtactgggctccggcaaaactctgaccatccaggtgaaggagttcggtgatgctggacaatacacctgccataagggcggcgaggttcttagccattctctgctgctcctccacaagaaggaagacggtatttggtcgaccgacatcctgaaggaccagaaggagcccaaaaacaagacttttttgcgctgcgaggccaaaaactactccggtcgcttcacatgctggtggctaacgaccatctcaaccgacctgaccttctctgtcaagagctctcgtggctccagcgatcctcagggcgtcacctgcggggcagccaccctgagcgcagagcgcgtgagaggggacaacaaggagtatgagtactcggtggagtgtcaggaggactcggcctgcccagcggccgaggagagcctgcccattgaggtgatggtagacgccgtccacaagctgaagtacgagaactacacctcctccttctttatccgggacatcatcaagcccgacccgcccaagaacctgcagctcaagcctttgaagaattcccgccaggttgaagtcagttgggagtaccctgacacctggtctactccgcacagctatttctcgctgaccttctgcgtgcaggtccagggaaagtccaagcgcgagaagaaagacagggtgttcaccgacaagacctccgccaccgtgatttgccgaaaaaacgcctccatctccgtgcgtgcgcaggaccgctactacagttcctcttggtccgagtgggcttccgtgccttgttcaggcagtggagagggcagaggaagtctgctaacatgcggtgacgtcgaggagaatcctggcccaatggctctgctgctggcactgagtctgctggtcctgtggacatcacctgcacctaccctgagtggaactaatgacgctgaagattgttgcctgagcgtgacccagaagccaatccccggctacatcgtgaggaacttccactatctgctgatcaaggacggatgcagggtgcctgcagtggtgtttaccacactgcggggcagacagctgtgcgcaccacctgatcagccctgggtcgagagaattattcagagactgcagcgcacctcagctaagatgaaacgccgctcaagttaa

SEQ ID NO: 44 - protein, 865 aa, Artificial Sequence
  mus261M CAR amino acid
MASPLTRFLSLNLLLLGESIILGSGEAKPQAQVQLEESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYNYWGQGTQVTVSSTSTTTKPVLRTPSPVHPTGTSQPQRPEDCRPRGSVKGTGLDFACDIYIWAPLAGICVALLLSLIITLICSVLKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGATNFSLLKQAGDVEENPGPMCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSGEGRGSLLTCGDVEENPGPMAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPVS

SEQ ID NO: 45 - DNA, 2598 bp, Artificial Sequence
  mus261M CAR nucleic acid
atggcctcaccgttgacccgctttctgtcgctgaacctgctgctgctgggtgagtcgattatcctggggagtggagaagctaagccacaggcacaggtgcagctggaggagtctggaggaggaagcgtgcaagtgggaggctccctgaggctgtcttgcgcagcaagcggatacagatccagcgtgtgcatgggatggtttagacaggcaccaggaaaggagagggagagagtggcagtgatcggaagggacggatccaccacatatatcgattctgtgaagggccggtttaccatctccagagactctgccaagaacacactgtctctgcagatggacaatctgaagcctgaagacacagccatgtacagctgcgccgccggcctgggatactgggcttgcgaatacaactactgggggcagggcactcaggtgaccgtctcttctactagtactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctctgtgctcaaatggatcaggaaaaaattcccccacatattcaagcaaccatttaagaagaccactggagcagctcaagaggaagatgcttgtagctgccgatgtccacaggaagaagaaggaggaggaggaggctatgagctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgcggcagcggagctacaaacttcagtcttctaaaacaggctggtgacgtggaggagaatcccggccctatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcagtggagagggcagaggaagtctgctaacatgcggtgacgtcgaggagaatcctggcccaatggctcctagagtgacacctttgctggccttttctcttctagtgttgtggacgtttcctgcacccaccctgggtggagcgaatgatgcagaggactgctgcctcagtgtcacccaaaggcccataccagggaacattgtgaaggccttccgctatcttctcaatgaagatggatgtcgggttcctgctgtggtcttcactactttacgcggctaccagctgtgtgctcctccagaccagccctgggtggaccgtatcatccgaaggctgaagaaaagcagcgccaagaacaaaggcaactcaacaagaagatccccagtttcctaa

SEQ ID NO: 46 - protein, 5 aa, Artificial Sequence
  Glycine-serine polymer linker
  MISC_FEATURE (1)..(5): GSGGS can be repeated n times, where n is an integer of at least one
GSGGS

SEQ ID NO: 47 - protein, 4 aa, Artificial Sequence
  Glycine-serine polymer linker
  MISC_FEATURE (1)..(4): GGGS can be repeated n times, where n is an integer of at least one
GGGS

SEQ ID NO: 48 - protein, 5 aa, Artificial Sequence
  Glycine-serine polymer linker
  MISC_FEATURE (1)..(5): GGGGS can be repeated n times, where n is an integer of at least one
GGGGS